(19) United States
(12) Patent Application Publication
(10) Pub. No.: US 2023/0183732 A1
(43) Pub. Date: Jun. 15, 2023

(54) STALK ROT-RESISTANT MAIZE PLANTS

(71) Applicants: AgReliant Genetics, LLC, Westfield, IN (US); Limagrain Europe S.A., Saint-Beauzire (FR); KWS SAAT SE & Co. KGaA, Einbeck (DE)

(72) Inventors: David B. WILLMOT, Westfield, IN (US); Brian D. FOSS, Westfield, IN (US); James M. JOHNSON, Whitestown, IN (US); Jordi Comadran TRABAL, Riom (FR); Daniela SCHEUERMANN, Einbeck (DE); Monika KLOIBER-MAITZ, Einbeck (DE)

(21) Appl. No.: 17/924,895

(22) PCT Filed: May 11, 2021
(86) PCT No.: PCT/US2021/031829

§ 371 Date: Nov. 11, 2022

Related U.S. Application Data
(60) Provisional application No. 63/022,868, filed on May 11, 2020.

(51) Int. Cl. C12N 15/82; C12Q 1/6895; C07K 14/415

(52) U.S. Cl. CPC C12N 15/8282; C12Q 1/6895; C07K 14/415; C12Q 2600/13; C12Q 2600/156

(57) ABSTRACT

The present disclosure is in the field of plant breeding and disease resistance. A method for the development of a corn plant enhanced for resistance to Colletotrichum graminicola and secondarily to Fusarium spp., both of which incite stalk rot disease, is provided. Also provided is a method to identify corn plants with polynucleotide sequences identified to serve as diagnostic markers for resistance to these pathogens. Further described is the introgression of desired genetic material from one or more parent plants into progeny with precision and accuracy to increase their resistance to these diseases with minimal linkage drag from the donor genome.

### CROSS-REFERENCE TO RELATED APPLICATIONS

This application claims the benefit of priority to U.S. Provisional Application No. 63/022,868 filed on May 11, 2020, which is hereby incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: AGR-002_SeqList_20210507_6_ST25.txt, date created: May 7, 2021, file size≈0.99 MB.

### FIELD

This disclosure relates to compositions and methods useful in identifying and selecting for plants with pathogen resistance. Additionally, the disclosure relates to plants that have been genetically transformed or introgressed with compositions of the disclosure.

### BACKGROUND

Stalk rot of corn is a disease complex incited by several pathogens including Colletotrichum graminicola Ces. Wils., Fusarium verticilliodes, and related species. These pathogens incite anthracnose stalk rot (ASR) and Fusarium stalk rot, respectively. These stalk rots alone are estimated to have reduced the U.S.A. corn crop by 463.4 million bushels (11.8 million MT) in yield in 2016. See, Mueller et al., “Corn yield loss estimates due to diseases in the United States and Ontario, Canada from 2012 to 2015”, Papers in Plant Pathology, 2016, University of Nebraska—Lincoln. Stalk rot also causes extensive stalk lodging. See, Callaway et al., “Effect of anthracnose stalk rot on grain yield and related traits of maize adapted to the northeastern United States.” Canadian Journal of Plant Science, 1992, 72(4), 1031-1036. Others have cited sources of resistance against ASR. See, Badu-Apraku et al., “A major gene for resistance to stalk rot in maize.”, 1987, Phytopathology 77:957-959; Toman & White, “Inheritance of resistance to stalk rot of corn.” Phytopathology, 1993, 83:981-986; Jung el al., “Generation-means analysis and quantitative trait locus mapping of anthracnose stalk rot genes in maize.” TAG, 1994, 89:413-418.

The known resistance genes are not sufficient for the development and production of varieties with durable resistance. Cultivation of corn year by year in the same fields, as often practiced in many regions of the world, significantly increases disease severity and the tendency of pathogens to break host resistance. There is a need for new resistance sources and combinations of resistance genes to overcome resistance-breaking variants of the pathogens. The need for new and alternative sources for resistance to plant diseases, in particular to anthracnose stalk rot, is met by the subject matter disclosed in this application.

### BRIEF DESCRIPTION OF THE DRAWINGS

FIG. 1 shows a Manhattan plot of QTL results showing two ASR QTL on chromosomes 4 and 6 surpassing the empirical LOD (log of odds) significance threshold (P<0.01).

FIG. 2 shows a comparison of amplicons within the QTL on chromosome 4. Genotype 1: Mp305, described in U.S. Pat. No. 8,062,847, incorporated by reference herein in its entirety; Genotype 2: DW1035, BC5 selecting for Mp305 chromosome 4 fragment; Genotype 3: NC262A, source of the present disclosure; Genotype 4: NC342, full sib source of the present disclosure; Genotype 5: GEMN-0117, ASR tolerant check; Genotype 6: GEMS-0016, ASR tolerant check; Genotype 7: MN13, susceptible check; Genotype 8: MM69, susceptible check; Genotype 9: KW7638, susceptible check; Genotype 10: CB1, susceptible check.

FIG. 3 shows an exemplary stalk splitter. The stalk splitter has 50 cm handles for ergonomic ease, a blade above the body of the mechanism, and spring tensioned rollers below the blade which center the split stalk as it comes up through the body.

FIG. 4 shows how the exemplary stalk splitter provides a centered cut with visibility to the stalk being cut thereby revealing the degree of stalk rot inside.

FIG. 5 is a closeup view of the body of the splitter and its aluminum body with slots for the centering rollers to expand into.

FIG. 6 is a top view showing the central hole up through which the stalk emerges. The stalk is split by the cross blade as pressure is exerted downward to the ground by the handles.

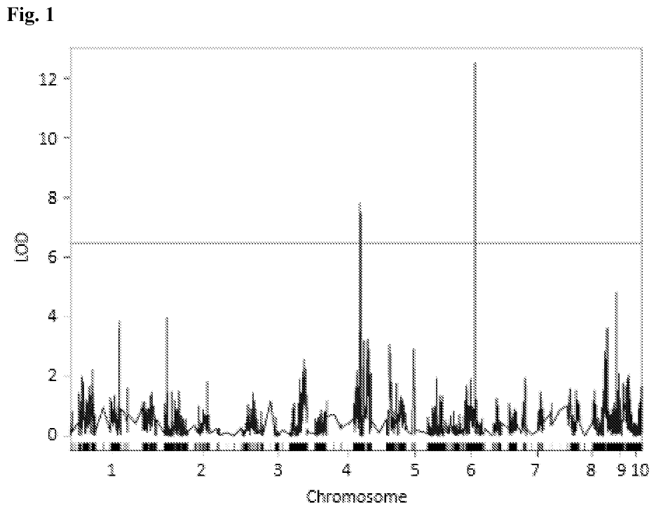

### SEQUENCES

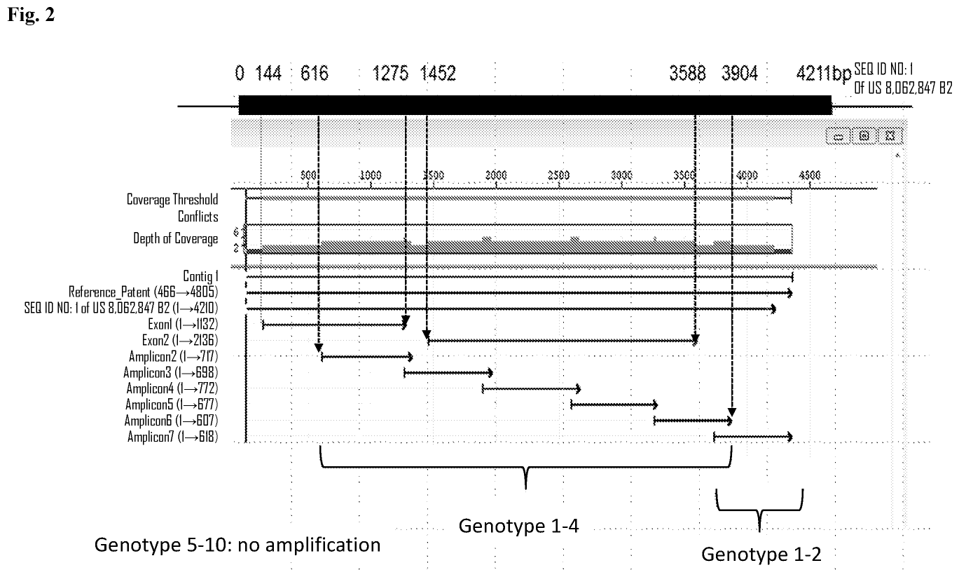

The sequences described in this application are summarized in the following table.

| SEQ ID NO: | Description |
|---|---|
| 1 | genomic DNA of Cesa2 |
| 2 | cDNA of transcript 1 derived from genomic DNA of Cesa2 |
| 3 | cDNA of transcript 2 derived from genomic DNA of Cesa2 |
| 4 | cDNA of transcript 3 derived from genomic DNA of Cesa2 |
| 5 | cDNA of transcript 4 derived from genomic DNA of Cesa2 |
| 6 | cDNA of transcript 5 derived from genomic DNA of Cesa2 |
| 7 | cDNA of transcript 6 derived from genomic DNA of Cesa2 |
| 8 | cDNA of transcript 7 derived from genomic DNA of Cesa2 |
| 9 | cDNA of transcript 8 derived from genomic DNA of Cesa2 |
| 10 | cDNA of transcript 9 derived from genomic DNA of Cesa2 |
| 11 | Cesa2 protein encoded by transcript 1 |
| 12 | Cesa2 protein encoded by transcript 2 |
| 13 | Cesa2 protein encoded by transcript 3 |
| 14 | Cesa2 protein encoded by transcript 4 |
| 15 | Cesa2 protein encoded by transcript 5 |
| 16 | Cesa2 protein encoded by transcript 6 |
| 17 | Cesa2 protein encoded by transcript 7 |
| 18 | Cesa2 protein encoded by transcript 8 |
| 19 | Cesa2 protein encoded by transcript 9 |
| 20 | genomic DNA of CDPK3 |
| 21 | cDNA of CDPK3 |
| 22 | CDPK3 protein |
| 23 | genomic DNA of Rpp13 |
| 24 | cDNA of transcript 1 derived from genomic DNA of Rpp13 |
| 25 | cDNA of transcript 2 derived from genomic DNA of Rpp13 |
| 26 | cDNA of transcript 3 derived from genomic DNA of Rpp13 |
| 27 | cDNA of transcript 4 derived from genomic DNA of Rpp13 |
| 28 | cDNA of transcript 5 derived from genomic DNA of Rpp13 |
| 29 | cDNA of transcript 6 derived from genomic DNA of Rpp13 |
| 30 | cDNA of transcript 10 derived from genomic DNA of Rpp13 |
| 31 | cDNA of transcript 11 derived from genomic DNA of Rpp13 |
| 32 | cDNA of transcript 12 derived from genomic DNA of Rpp13 |
| 33 | cDNA of transcript 13 derived from genomic DNA of Rpp13 |
| 34 | Rpp13 protein encoded by transcript 1 |
| 35 | Rpp13 protein encoded by transcript 2 |
| 36 | Rpp13 protein encoded by transcript 3 |
| 37 | Rpp13 protein encoded by transcript 4 |
| 38 | Rpp13 protein encoded by transcript 5 |
| 39 | Rpp13 protein encoded by transcript 6 |
| 40 | Rpp13 protein encoded by transcript 10 |
| 41 | Rpp13 protein encoded by transcript 11 |
| 42 | Rpp13 protein encoded by transcript 12 |
| 43 | Rpp13 protein encoded by transcript 13 |
| 44 | genomic DNA of GSO1 |
| 45 | cDNA of GSO1 |
| 46 | GSO1 protein |
| 47 | Genomic DNA of LRX4 |
| 48 | cDNA of LRX4 |
| 49 | LRX4 protein |
| 50 | reference rcg1 sequence as disclosed in SEQ ID NO: 1 of U.S. Pat. No. 8,062,847 B2 |
| 51 | marker PZE-104115065 |
| 52 | marker Mc0241 (forward primer) |
| 53 | marker Mc0241 (reverse primer) |
| 54 | marker Bnl3.03 (forward primer) |
| 55 | marker Bnl3.03 (reverse primer) |
| 56 | marker Affx-90199961 |
| 57 | marker Affx-90852574 (=PZE-106073551) |
| 58 | primer_alleleX for marker for detecting SNP at position 413 referred to rcg1 as |
| | disclosed in U.S. Pat. No. 8,062,847 B2 |
| 59 | primer_alleleY for marker for detecting SNP at position 413 referred to rcg1 as |
| | disclosed in U.S. Pat. No. 8,062,847 B2 |
| 60 | primer common for marker for detecting SNP at position 413 referred to rcg1 as |
| | disclosed in U.S. Pat. No. 8,062,847 B2 |
| 61 | primer_alleleX for marker for detecting SNP at position 1099 referred to rcg1 as |
| | disclosed in U.S. Pat. No. 8,062,847 B2 |
| 62 | primer_alleleY for marker for detecting SNP at position 1099 referred to rcg1 as |
| | disclosed in U.S. Pat. No. 8,062,847 B2 |
| 63 | primer common for marker for detecting SNP at position 1099 referred to rcg1 as |
| | disclosed in U.S. Pat. No. 8,062,847 B2 |
| 64 | primer_alleleX for marker for detecting SNP at position 1250 referred to rcg1 as |
| | disclosed in U.S. Pat. No. 8,062,847 B2 |
| 65 | primer_alleleY for marker for detecting SNP at position 1250 referred to rcg1 as |
| | disclosed in U.S. Pat. No. 8,062,847 B2 |
| 66 | primer common for marker for detecting SNP at position 1250 referred to rcg1 as |
| | disclosed in U.S. Pat. No. 8,062,847 B2 |
| 67 | primer_alleleX for marker for detecting SNP at position 1607 referred to rcg1 as |
| | disclosed in U.S. Pat. No. 8,062,847 B2 |
| 68 | primer_alleleY for marker for detecting SNP at position 1607 referred to rcg1 as |
| | disclosed in U.S. Pat. No. 8,062,847 B2 |
| 69 | primer common for marker for detecting SNP at position 1607 referred to rcg1 as |
| | disclosed in U.S. Pat. No. 8,062,847 B2 |
| 70 | primer_alleleX for marker for detecting SNP at position 2001 referred to rcg1 as |
| | disclosed in U.S. Pat. No. 8,062,847 B2 |
| 71 | primer_alleleY for marker for detecting SNP at position 2001 referred to rcg1 as |
| | disclosed in U.S. Pat. No. 8,062,847 B2 |
| 72 | primer common for marker for detecting SNP at position 2001 referred to rcg1 as |
| | disclosed in U.S. Pat. No. 8,062,847 B2 |
| 73 | primer_alleleX for marker for detecting SNP at position 2598 referred to rcg1 as |
| | disclosed in U.S. Pat. No. 8,062,847 B2 |
| 74 | primer_alleleY for marker for detecting SNP at position 2598 referred to rcg1 as |
| | disclosed in U.S. Pat. No. 8,062,847 B2 |
| 75 | primer common for marker for detecting SNP at position 2598 referred to rcg1 as |
| | disclosed in U.S. Pat. No. 8,062,847 B2 |
| 76 | primer_alleleX for marker for detecting SNP at position 3342 referred to rcg1 as |
| | disclosed in U.S. Pat. No. 8,062,847 B2 |
| 77 | primer_alleleY for marker for detecting SNP at position 3342 referred to rcg1 as |
| | disclosed in U.S. Pat. No. 8,062,847 B2 |
| 78 | primer common for marker for detecting SNP at position 3342 referred to rcg1 as |
| | disclosed in U.S. Pat. No. 8,062,847 B2 |
| 79 | primer_alleleX for marker for detecting SNP C12305-001-K1 |
| 80 | primer_alleleY for marker for detecting SNP C12305-001-K1 |
| 81 | primer common for marker for detecting SNP C12305-001-K1 |
| 82 | primer_alleleX for marker for detecting SNP C12307-001-K1 |
| 83 | primer_alleleY for marker for detecting SNP C12307-001-K1 |
| 84 | primer common for marker for detecting SNP C12307-001-K1 |
| 85 | primer_alleleX for marker for detecting SNP C16759-001-K1 |
| 86 | primer_alleleY for marker for detecting SNP C16759-001-K1 |
| 87 | primer common for marker for detecting SNP C16759-001-K1 |
| 88 | primer_alleleX for marker for detecting SNP C16760-001-K1 |
| 89 | primer_alleleY for marker for detecting SNP C16760-001-K1 |
| 90 | primer common for marker for detecting SNP C16760-001-K1 |
| 91 | primer_alleleX for marker for detecting SNP C12314-001-K1 |
| 92 | primer_alleleY for marker for detecting SNP C12314-001-K1 |
| 93 | primer common for marker for detecting SNP C12314-001-K1 |
| 94 | amplicon 2 according to FIG. 2 |
| 95 | Forward primer of primer pair 1 for generating amplicon 2 |
| 96 | Reverse primer of primer pair 1 for generating amplicon 2 |
| 97 | Forward primer of primer pair 2 for generating amplicon 2 |
| 98 | Reverse primer of primer pair 2 for generating amplicon 2 |
| 99 | Forward primer of primer pair 3 for generating amplicon 2 |
| 100 | Reverse primer of primer pair 3 for generating amplicon 2 |
| 101 | amplicon 3 according to FIG. 2 |
| 102 | Forward primer of primer pair 1 for generating amplicon 3 |
| 103 | Reverse primer of primer pair 1 for generating amplicon 3 |
| 104 | Forward primer of primer pair 3 for generating amplicon 3 |
| 105 | Reverse primer of primer pair 3 for generating amplicon 3 |
| 106 | amplicon 4 according to FIG. 2 |
| 107 | Forward primer of primer pair 3 for generating amplicon 4 |
| 108 | Reverse primer of primer pair 3 for generating amplicon 4 |
| 109 | amplicon 5 according to FIG. 2 |
| 110 | Forward primer of primer pair 1 for generating amplicon 5 |
| 111 | Reverse primer of primer pair 1 for generating amplicon 5 |
| 112 | Forward primer of primer pair 2 for generating amplicon 5 |
| 113 | Reverse primer of primer pair 2 for generating amplicon 5 |
| 114 | amplicon 6 according to FIG. 2 |
| 115 | Forward primer of primer pair 1 for generating amplicon 6 |
| 116 | Reverse primer of primer pair 1 for generating amplicon 6 |
| 117 | Forward primer of primer pair 3 for generating amplicon 6 |
| 118 | Reverse primer of primer pair 3 for generating amplicon 6 |
| 119 | amplicon 7 according to FIG. 2 |
| 120 | Forward primer of primer pair 1 for generating amplicon 7 |
| 121 | Reverse primer of primer pair 1 for generating amplicon 7 |
| 122 | Forward primer of primer pair 2 for generating amplicon 7 |
| 123 | Reverse primer of primer pair 2 for generating amplicon 7 |
| 124 | Forward primer of primer pair 3 for generating amplicon 7 |
| 125 | Reverse primer of primer pair 3 for generating amplicon 7 |
| 126 | PZE-106066955 |
| 127 | PZE.106072681 |
| 128 | PZE.106073001 |
| 129 | PUT.163a.18167596.1294 |
| 130 | ZmSYNBREED_54943_173 |
| 131 | ZmSYNBREED_54946_787 |
| 132 | PZE.106073260 |
| 133 | ZmSYNBREED_54949_671 |
| 134 | ZmSYNBREED_54956_986 |
| 135 | ZmSYNBREED_54966_239 |
| 136 | PZE.106073623 |
| 137 | ZmSYNBREED_54969_202 |
| 138 | ZmSYNBREED_54969_513 |
| 139 | ZmSYNBREED_54973_806 |
| 140 | ZmSYNBREED_54975_531 |
| 141 | ZmSYNBREED_54977_728 |
| 142 | ZmSYNBREED_54978_371 |
| 143 | PZE.106074105 |
| 144 | ZmSYNBREED_54980_496 |
| 145 | ZmSYNBREED_54981_535 |
| 146 | PZE.106074333 |
| 147 | ZmSYNBREED_54987_542 |
| 148 | PZE.106074560 |
| 149 | genomic DNA of Zm00001d037635 |
| 150 | cDNA of Zm00001d037635 |
| 151 | protein encoded by Zm00001d037635 |
| 152 | genomic DNA of Zm00001d037637 |
| 153 | cDNA of Zm00001d037637 |
| 154 | protein encoded by Zm00001d037637 |
| 155 | genomic DNA of Zm00001d037640 |
| 156 | cDNA of Zm00001d037640 |
| 157 | protein encoded by Zm00001d037640 |
| 158 | genomic DNA of Zm00001d037642 |
| 159 | cDNA of Zm00001d037642 |
| 160 | protein encoded by Zm00001d037642 |
| 161 | genomic DNA of Zm00001d037643 |
| 162 | cDNA of Zm00001d037643 |
| 163 | protein encoded by Zm00001d037643 |
| 164 | genomic DNA of Zm00001d037647 |
| 165 | cDNA of Zm00001d037647 |
| 166 | protein encoded by Zm00001d037647 |
| 167 | genomic DNA of Zm00001d037650 |
| 168 | cDNA of Zm00001d037650 |
| 169 | protein encoded by Zm00001d037650 |
| 170 | genomic DNA of Zm00001d037651 |
| 171 | cDNA of Zm00001d037651 |
| 172 | protein encoded by Zm00001d037651 |
| 173 | genomic DNA of Zm00001d037652 |
| 174 | cDNA of Zm00001d037652 |
| 175 | protein encoded by Zm00001d037652 |
| 176 | genomic DNA of Zm00001d037653 |
| 177 | cDNA of Zm00001d037653 |
| 178 | protein encoded by Zm00001d037653 |
| 179 | genomic DNA of Zm00001d037656 |
| 180 | cDNA of Zm00001d037656 |
| 181 | protein encoded by Zm00001d037656 |
| 182 | genomic DNA of Zm00001d037658 |
| 183 | cDNA of Zm00001d037658 |
| 184 | protein encoded by Zm00001d037658 |
| 185 | genomic DNA of Zm00001d037659 |
| 186 | cDNA of Zm00001d037659 |
| 187 | protein encoded by Zm00001d037659 |
| 188 | genomic DNA of Zm00001d037661 |
| 189 | cDNA of Zm00001d037661 |
| 190 | protein encoded by Zm00001d037661 |
| 191 | genomic DNA of Zm00001d037663 |
| 192 | cDNA of Zm00001d037663 |
| 193 | protein encoded by Zm00001d037663 |
| 194 | genomic DNA of Zm00001d037668 |
| 195 | cDNA of Zm00001d037668 |
| 196 | protein encoded by Zm00001d037668 |
| 197 | genomic DNA of Zm00001d037672 |
| 198 | cDNA of Zm00001d037672 |
| 199 | protein encoded by Zm00001d037672 |
| 200 | genomic DNA of Zm00001d037674 |
| 201 | cDNA of Zm00001d037674 |
| 202 | protein encoded by Zm00001d037674 |
| 203 | genomic DNA of Zm00001d037675 |
| 204 | cDNA of Zm00001d037675 |
| 205 | protein encoded by Zm00001d037675 |
| 206 | genomic DNA of AGP9L |
| 207 | cDNA of AGP9L |
| 208 | protein encoded by AGP9L |
| 209 | genomic DNA of Zm00001d037635 - NC262A genotype |
| 210 | cDNA of Zm00001d037635 - NC262A genotype |
| 211 | protein encoded by Zm00001d037635 - NC262A genotype |
| 212 | genomic DNA of Cesa2 - NC262A genotype |
| 213 | cDNA of Cesa2 - NC262A genotype |
| 214 | protein encoded by Cesa2 - NC262A genotype |
| 215 | genomic DNA of Zm00001d037637 - NC262A genotype |
| 216 | cDNA of Zm00001d037637 - NC262A genotype |
| 217 | protein encoded by Zm00001d037637 - NC262A genotype |
| 218 | genomic DNA of Zm00001d037640 - NC262A genotype |
| 219 | cDNA of Zm00001d037640 - NC262A genotype |
| 220 | protein encoded by Zm00001d037640 - NC262A genotype |
| 221 | genomic DNA of Zm00001d037643 - NC262A genotype |
| 222 | cDNA of Zm00001d037643 - NC262A genotype |
| 223 | protein encoded by Zm00001d037643 - NC262A genotype |
| 224 | genomic DNA of Zm00001d037647 - NC262A genotype |
| 225 | cDNA of Zm00001d037647 - NC262A genotype |
| 226 | protein encoded by Zm00001d037647 - NC262A genotype |
| 227 | genomic DNA of Rpp13 - NC262A genotype |
| 228 | cDNA of Rpp13 - NC262A genotype |
| 229 | protein encoded by Rpp13 - NC262A genotype |
| 230 | genomic DNA of Zm00001d037650 - NC262A genotype |
| 231 | cDNA of Zm00001d037650 - NC262A genotype |
| 232 | protein encoded by Zm00001d037650 - NC262A genotype |
| 233 | genomic DNA of Zm00001d037651 - NC262A genotype |
| 234 | cDNA of Zm00001d037651 - NC262A genotype |
| 235 | protein encoded by Zm00001d037651 - NC262A genotype |
| 236 | genomic DNA of Zm00001d037653 - NC262A genotype |
| 237 | cDNA of Zm00001d037653 - NC262A genotype |
| 238 | protein encoded by Zm00001d037653 - NC262A genotype |
| 239 | genomic DNA of Zm00001d037658 - NC262A genotype |
| 240 | cDNA of Zm00001d037658 - NC262A genotype |
| 241 | protein encoded by Zm00001d037658 - NC262A genotype |
| 242 | genomic DNA of Zm00001d037659 - NC262A genotype |
| 243 | cDNA of Zm00001d037659 - NC262A genotype |
| 244 | protein encoded by Zm00001d037659 - NC262A genotype |
| 245 | genomic DNA of Zm00001d037663 - NC262A genotype |
| 246 | cDNA of Zm00001d037663 - NC262A genotype |
| 247 | protein encoded by Zm00001d037663 - NC262A genotype |
| 248 | genomic DNA of GSO1 - NC262A genotype |
| 249 | cDNA of GSO1 - NC262A genotype |
| 250 | GSO1 protein - NC262A genotype |
| 251 | genomic DNA of Zm00001d037668 - NC262A genotype |
| 252 | cDNA of Zm00001d037668 - NC262A genotype |
| 253 | protein encoded by Zm00001d037668 - NC262A genotype |
| 254 | genomic DNA of Zm00001d037672 - NC262A genotype |
| 255 | cDNA of Zm00001d037672 - NC262A genotype |
| 256 | protein encoded by Zm00001d037672 - NC262A genotype |
| 257 | genomic DNA of Zm00001d037674 - NC262A genotype |
| 258 | cDNA of Zm00001d037674 - NC262A genotype |
| 259 | protein encoded by Zm00001d037674 - NC262A genotype |
| 260 | genomic DNA of Zm00001d037675 - NC262A genotype |
| 261 | cDNA of Zm00001d037675 - NC262A genotype |
| 262 | protein encoded by Zm00001d037675 - NC262A genotype |
| 263 | genomic DNA of LRX4 - NC262A genotype |
| 264 | cDNA of LRX4 - NC262A genotype |
| 265 | LRX4 protein - NC262A genotype |
| 266 | genomic DNA of Zm00001e031194 NC262A genotype |
| | ZmNC262Av2c_OGOO1508HC.1_genomic sequence |
| 267 | cDNA encoded by ZmNC262Av2c_OGOO1508HC.1 |
| 268 | protein encoded by ZmNC262Av2c_OGOO1508HC.1_pro |
| 269 | genomic DNA of Zm00001e031197 NC262A genotype |
| | ZmNC262Av2c_OGOO1512HC.1 genomic sequence |
| 270 | cDNA encoded by ZmNC262Av2c_OGOO1512HC.1 |
| 271 | protein encoded by ZmNC262Av2c_OGOO1512HC.1 |
| 272 | Genomic sequence of contig 81 NC262A genotype NC262A.contig81: 29672100- |
| | 29819600 |

### SUMMARY

In one aspect is provided a process of identifying a maize plant that displays enhanced resistance to anthracnose stalk rot, the process comprising detecting in the maize plant
a. the presence of at least two markers at a resistance locus on chromosome 6 comprising a “G” at C16759-001-K1 and one of the following single nucleotide polymorphisms:
   a “C” at C12305-001-K1,
   a “C” at C12307-001-K1,
   a “G” at C16760-001-K1,
   an “A” at C12314-001-K1; and/or
b. the presence of at least one marker at a resistance locus on chromosome 6 comprising at least one of the variant nucleotide polymorphisms recited in Table 13; and/or
c. the presence of at least one marker at a resistance locus on chromosome 4, wherein the resistance locus comprises a Rcg1 resistance allele having a haplotype comprising one or more single nucleotide polymorphism selected from the group consisting of:
   a “C” at position 413 referenced to SEQ ID NO: 50,
   a “C” at position 958 referenced to SEQ ID NO: 50,
   a “C” at position 971 referenced to SEQ ID NO: 50,
   a “T” at position 1099 referenced to SEQ ID NO: 50,
   an “A” at position 1154 referenced to SEQ ID NO: 50,
   a “T” at position 1250 referenced to SEQ ID NO: 50,
   a “G” at position 1607 referenced to SEQ ID NO: 50,
   a “G” at position 2001 referenced to SEQ ID NO: 50,
   an “A” at position 2598 referenced to SEQ ID NO: 50, and
   an “A” at position 3342 referenced to SEQ ID NO: 50,
      where the at least two markers of (a) or the at least one marker of (b) are/is closely linked to and associated with the resistance locus on chromosome 6 and the at least one marker of (c) is closely linked to and associated with the resistance locus on chromosome 4. Preferably, Rcg1 resistance allele is distinct at each position from the Rcg1 allele derived from accession Mp305 (see U.S. Pat. No. 8,062,847).

In some embodiments, the resistance locus on chromosome 6 is located on a chromosomal interval between makers PZE-106066805 and PZE-106075546. In some embodiments, the resistance locus on chromosome 4 is located on a chromosomal interval between makers PZE-104102206 and PZE-104132759. In other embodiments, the resistance locus on chromosome 6 is located on a chromosomal interval between 139646631 and 139889078 numbered according to the B73AGPv05 genome sequence. In some embodiments the resistance locus of chromosome 6 comprises SEQ ID NO: 272, or a fragment thereof.

In some embodiments, the resistance locus on chromosome 6 comprises one or more nucleotide sequences selected from the group consisting of
i. a nucleotide sequence of SEQ ID NO: 266 or 269,
ii. a nucleotide sequence having the coding sequence of SEQ ID NO: 267 or 270,
iii. a nucleotide sequence which is complementary to a nucleotide sequence from i. or ii.,
iv. a nucleotide sequence with at least 80% or 85%, preferably at least 90%, 91%, 92%, 93%, 94% or 95%, more preferably at least 96%, 97%, 98% or 99% identity to a nucleotide sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 268 or 271, or
viii. a nucleotide sequence which encodes for a protein comprising a sequence with at least 80% or 85%, preferably at least 90%, 91%, 92%, 93%, 94% or 95%, more preferably at least 96%, 97%, 98% or 99% identity to the sequence of SEQ ID NO: 268 or 271.

In some embodiments, the resistance locus on chromosome 4 does not produce the amplicon according to SEQ ID NO: 119 upon polymerase chain reaction amplification using primers of SEQ ID NOS: 120 and 121, or primers of SEQ ID NOS: 122 and 123, or primers of SEQ ID NOS: 124 and 125.

In some embodiments, the resistance locus on chromosome 4 does not produce an amplicon selected from the group consisting of SEQ ID NOs: 94, 101, 106, 109, and 114 upon polymerase chain reaction amplification using primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118.

In some embodiments, the resistance locus on chromosome 6 is derived from NC262A. In some embodiments, the resistance locus on chromosome 4 is derived from NC262A or NC342.

In some embodiments, the process comprises detecting in the maize plant the presence or absence of at least one allele at the resistance locus on chromosome 6 as defined above under b. In a specific embodiment, the at least one marker at the resistance locus on chromosome 6 detects a “G” at C16759-001-K1.

In some embodiments, the process comprises detecting in the maize plant both (A) the presence or absence of at least one allele at the resistance locus on chromosome 6 as defined above under b. and (B) the presence or absence of at least one marker at the resistance locus on chromosome 4 as defined above under c. In a specific embodiment, the at least one marker at the resistance locus on chromosome 6 detects a “G” at C16759-001-K1. In a specific embodiment, the at least one marker at the resistance locus on chromosome 4 detects the single nucleotide polymorphisms of the “C” at position 413 in SEQ ID NO: 50, the “C” at position 958 in SEQ ID NO: 50, the “C” at position 971 in SEQ ID NO: 50, the “T” at position 1099 in SEQ ID NO: 50, the “A” at position 1154 in SEQ ID NO: 50, the “T” at position 1250 in SEQ ID NO: 50, the “G” at position 1607 in SEQ ID NO: 50, the “G” at position 2001 in SEQ ID NO: 50, the “A” at position 2598 in SEQ ID NO: 50, or the “A” at position 3342 in SEQ ID NO: 50. In a specific embodiment, the at least one marker at the resistance locus on chromosome 4 detects the single nucleotide polymorphisms of the “C” at position 413 in SEQ ID NO: 50.

In various embodiments, the presence or absence of at least one nucleotide polymorphism is detected by polymerase chain reaction amplification of a nucleic acid present in the maize plant with a primer configured to specifically amplify a nucleic acid sequence comprising one or more of the nucleotide polymorphisms. In a specific embodiment, the single nucleotide polymorphism is a “C” at position 413 in SEQ ID NO: 50, and the primer comprises the sequence GTACCATGTGACCA (SEQ ID NO: 406). In a specific embodiment, the single nucleotide polymorphism is a “T” at position 1099 in SEQ ID NO: 50, and the primer comprises the sequence GTAGTGTTTTGAC (SEQ ID NO: 407). In a specific embodiment, the single nucleotide polymorphism is a “T” at position 1250 in SEQ ID NO: 50, and the primer comprises the sequence TGATCTCAAAGAT (SEQ ID NO: 408). In a specific embodiment, the single nucleotide polymorphism is a “G” at position 1607 in SEQ ID NO: 50, and the primer comprises the sequence GTTATGTGCACAA (SEQ ID NO: 409). In a specific embodiment, the single nucleotide polymorphism is a “G” at position 2001 in SEQ ID NO: 50, and the primer comprises the sequence AGATGAAGGCTGT (SEQ ID NO: 410). In a specific embodiment, the single nucleotide polymorphism is a “A” at position 2598 in SEQ ID NO: 50, and the primer comprises the sequence AAGTGACATGCAG (SEQ ID NO: 411). In a specific embodiment, the single nucleotide polymorphism is a “A” at position 3342 in SEQ ID NO: 50, and the primer comprises the sequence CATCTGATGAAAGC (SEQ ID NO: 412). In some embodiments, the nucleotide polymorphisms are selected from the group consisting of the variant nucleotides of Table 13.

In various embodiments, the presence or absence of the allele comprising a “G” at C16759-001-K1 is detected by polymerase chain reaction amplification of a nucleic acid present in the maize plant with a primer configured to specifically amplify a nucleic acid sequence of the allele.

In a specific embodiment, the primer comprises the sequence AATTATGCTGATGA (SEQ ID NO: 413).

In another aspect is provided a process for selecting a maize plant with anthracnose stalk rot resistance, the process comprising identifying the maize plant according to any of the above processes, and selecting the maize plant as having anthracnose stalk rot resistance if the presence or absence of the at least one marker at the resistance locus on chromosome 6 and/or the at least one marker at the resistance locus on chromosome 4 is detected. In some embodiments, the process further comprises selecting the maize plant that comprises at least one additional marker allele that is closely linked to and associated with the nucleotide polymorphism(s). In a specific embodiment, the additional marker allele is linked to the single nucleotide polymorphism by no more than 2 cM on a single meiosis based genetic map. In various embodiments, the process further comprises selecting the maize plant that comprises at least one additional marker allele that is linked to and associated with the allele comprising a “G” at C16759-001-K1. In a specific embodiment, the additional marker allele is linked to the allele comprising a “G” at C16759-001-K1 by no more than 2 cM on a single meiosis based genetic map. In some embodiments, the additional marker allele is linked to an allele comprising at least one of the variant nucleotide polymorphisms recited in Table 13.

In various embodiments of any of the above processes, the process further comprises backcrossing the identified maize plant with another maize plant, preferably comprising backcrossing the resistance locus on chromosome 6 into a genotype which is not NC262A and/or the resistance locus on chromosome 4 into a genotype which is not NC262A or NC342.

In another aspect is provided is a method of introgressing an allele associated with anthracnose stalk rot resistance into a maize plant, the method comprising:
a. screening a population with a nucleic acid assay for the detection of at least one marker at a resistance locus on chromosome 6 comprising
   (i) the following single nucleotide polymorphisms:
      a “C” at C12305-001-K1,
      a “C” at C12307-001-K1,
      a “G” at C16759-001-K1,
      a “G” at C16760-001-K1,
      an “A” at C12314-001-K1; and/or
   (ii) one or more of the variant nucleotide polymorphisms recited in Table 13; and
b. selecting from the population at least one maize plant comprising the resistance locus on chromosome 6 or comprising a “G” at C16759-001-K1 and/or one or more of the variant nucleotide polymorphisms recited in Table 13; and
c. crossing the at least one maize plant to a second maize plant;
d. evaluating progeny plants for the presence of the “G” at C16759-001-K1 and/or one or more of the variant nucleotide polymorphisms recited in Table 13; and
e. selecting progeny plants possessing the “G” at C16759-001-K1 and/or one or more of the variant nucleotide polymorphisms recited in Table 13.

In some embodiments, the at least one marker is located within 5 cM of the “G” at C16759-001-K1. In some embodiments, the at least one marker is located within 1 cM of the “G” at C16759-001-K1. In some embodiments, the resistance locus on chromosome 6 is located on a chromosomal interval between makers PZE-106066805 and PZE-106075546. In some embodiments, the resistance locus on chromosome 6 is located on a chromosomal interval between 139646631 and 139889078 numbered according to the B73AGPv05 genome sequence. In some embodiments, the resistance locus of chromosome 6 comprises SEQ ID NO: 272, or a fragment thereof.

In some embodiments, the resistance locus on chromosome 6 comprising one or more nucleotide sequences selected from the group consisting of
i. a nucleotide sequence of SEQ ID NO: 266 or 269,
ii. a nucleotide sequence having the coding sequence of SEQ ID NO: 267 or 270,
iii. a nucleotide sequence which is complementary to a sequence from i. or ii.,
iv. a nucleotide sequence which has at least 80% or 85%, preferably at least 90%, 91%, 92%, 93%, 94% or 95%, more preferably at least 96%, 97%, 98% or 99% identity with a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NOs: 268 or 271, or
viii. a nucleotide sequence which encodes for a protein comprising a sequence with at least 80% or 85%, preferably at least 90%, 91%, 92%, 93%, 94% or 95%, more preferably at least 96%, 97%, 98% or 99% sequence identity to a sequence of SEQ ID NO: 268 or 271.

In various embodiments, the resistance locus on chromosome 6 is derived from NC262A.

In another aspect is provided a method of introgressing a locus associated with anthracnose stalk rot resistance into a maize plant, the method comprising:
a. screening a population with at least one marker to determine if one or more maize plants from the population comprises the locus associated with anthracnose stalk rot resistance, wherein the screening comprises a nucleic acid assay for the detection of at least one marker at a resistance locus on chromosome 4, wherein the resistance locus comprises a Rcg1 resistance allele having a haplotype comprising one or more single nucleotide polymorphism selected from the group consisting of:
   a “C” at position 413 referenced to SEQ ID NO: 50,
   a “C” at position 958 referenced to SEQ ID NO: 50,
   a “C” at position 971 referenced to SEQ ID NO: 50,
   a “T” at position 1099 referenced to SEQ ID NO: 50,
   an “A” at position 1154 referenced to SEQ ID NO: 50,
   a “T” at position 1250 referenced to SEQ ID NO: 50,
   a “G” at position 1607 referenced to SEQ ID NO: 50,
   a “G” at position 2001 referenced to SEQ ID NO: 50,
   an “A” at position 2598 referenced to SEQ ID NO: 50, and
   an “A” at position 3342 referenced to SEQ ID NO: 50; and
b. selecting from the population at least one maize plant comprising said locus associated with anthracnose stalk rot resistance; and
c. crossing the at least one maize plant to a second maize plant;
d. evaluating progeny plants for the at least one marker associated with anthracnose stalk rot resistance; and
e. selecting progeny plants possessing the allele associated with anthracnose stalk rot resistance.

In various embodiments, the at least one marker is located within 5 cM of any one of: “C” at position 413 referenced to SEQ ID NO: 50, a “C” at position 958 referenced to SEQ ID NO: 50, a “C” at position 971 referenced to SEQ ID NO: 50, a “T” at position 1099 referenced to SEQ ID NO: 50, an “A” at position 1154 referenced to SEQ ID NO: 50, a “T” at position 1250 referenced to SEQ ID NO: 50, a “G” at position 1607 referenced to SEQ ID NO: 50, a “G” at position 2001 referenced to SEQ ID NO: 50, an “A” at position 2598 referenced to SEQ ID NO: 50, or an “A” at position 3342 referenced to SEQ ID NO: 50.

In various embodiments, the at least one marker is located within 1 cM of any one of: “C” at position 413 referenced to SEQ ID NO: 50, a “C” at position 958 referenced to SEQ ID NO: 50, a “C” at position 971 referenced to SEQ ID NO: 50, a “T” at position 1099 referenced to SEQ ID NO: 50, an “A” at position 1154 referenced to SEQ ID NO: 50, a “T” at position 1250 referenced to SEQ ID NO: 50, a “G” at position 1607 referenced to SEQ ID NO: 50, a “G” at position 2001 referenced to SEQ ID NO: 50, an “A” at position 2598 referenced to SEQ ID NO: 50, or an “A” at position 3342 referenced to SEQ ID NO: 50.

In some embodiments, the resistance locus on chromosome 4 is located on a chromosomal interval between makers PZE-104102206 and PZE-104132759. In some embodiments, the resistance locus on chromosome 4 does not produce an amplicon selected from the group consisting of SEQ ID NOs: 94, 101, 106, 109, and 114 upon polymerase chain reaction amplification by means of primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118.

In some embodiments, the resistance locus on chromosome 4 is derived from NC262A or NC342.

In another aspect is provided a method for selecting a maize plant that displays resistance to anthracnose stalk rot, the method comprising:

a. obtaining a first maize plant that comprises within its genome a haplotype comprising one or more of
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50,
x. an “A” at position 3342 referenced to SEQ ID NO: 50, and
xi. a “G” at C16759-001-K1; and
   b. crossing the first maize plant to a second maize plant;
   c. evaluating progeny plants for the haplotype in a. or at least one marker allele linked to and associated with the haplotype in b.; and
   d. selecting progeny plants that possess the haplotype in a.

In some embodiments, the first maize plant is obtained in (a) that comprises within its genome a haplotype comprising a “G” at C16759-001-K1 and one or more of
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50, and
x. an “A” at position 3342 referenced to SEQ ID NO: 50.

In another aspect is provided a nucleic acid molecule comprising one or more nucleotide sequences selected from the group consisting of
i. a nucleotide sequence of SEQ ID NO: 266 or 269,
ii. a nucleotide sequence having a coding sequence of SEQ ID NO: 267 or 270,
iii. a nucleotide sequence which is complementary to a sequence from i. or ii.,
iv. a nucleotide sequence with at least 80% or 85%, preferably at least 90%, 91%, 92%, 93%, 94% or 95%, more preferably at least 96%, 97%, 98% or 99% identity to a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 268 or 271, or
viii. a nucleotide sequence which encodes for a protein comprising a sequence with at least 80% or 85%, preferably at least 90%, 91%, 92%, 93%, 94% or 95%, more preferably at least 96%, 97%, 98% or 99% identity to the sequence of SEQ ID NO: 268 or 271.

In another aspect is provided a nucleic acid molecule encoding a Rcg1 resistance allele having a haplotype comprising one or more single nucleotide polymorphisms selected from the group consisting of:
a “C” at position 413 referenced to SEQ ID NO: 50,
a “C” at position 958 referenced to SEQ ID NO: 50,
a “C” at position 971 referenced to SEQ ID NO: 50,
a “T” at position 1099 referenced to SEQ ID NO: 50,
an “A” at position 1154 referenced to SEQ ID NO: 50,
a “T” at position 1250 referenced to SEQ ID NO: 50,
a “G” at position 1607 referenced to SEQ ID NO: 50,
a “G” at position 2001 referenced to SEQ ID NO: 50,
an “A” at position 2598 referenced to SEQ ID NO: 50, and
an “A” at position 3342 referenced to SEQ ID NO: 50,

where the nucleic acid molecule is encoding a polypeptide capable of conferring or increasing resistance to a plant disease caused by fungal pathogen in a plant in which the polypeptide is expressed.

In some embodiments, the nucleic acid molecule does not produce the amplicon according to SEQ ID NO: 119 (Amplicon 7) upon polymerase chain reaction amplification using primers of SEQ ID NOs: 120 and 121, primers of SEQ ID NOs: 122 and 123, or primers of SEQ ID NOs: 124 and 125. In some embodiments, the nucleic acid molecule does not produce an amplicon selected from the group consisting of SEQ ID NOs: 94, 101, 106, 109, and 114 upon polymerase chain reaction amplification by means of primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118.

In another aspect is provided an expression cassette comprising any of the above nucleic acid molecules, wherein the nucleic acid molecule is operatively linked to heterologous regulatory element, preferably to a heterologous promoter.

In another aspect is provided a method for conferring or increasing resistance to anthracnose stalk rot in a maize plant, the method comprising the following steps:
(a) introducing or introgressing into at least one cell of a maize plant any of the above nucleic acid molecules or expression cassettes;
(b) optionally regenerating or growing a plant from the at least one cell, and
(c) causing expression of the nucleic acid molecule in the plant.

In another aspect is provided a method for manufacturing a maize plant having anthracnose stalk rot resistance, comprising:
(a) introducing or introgressing into at least one cell of a maize plant the above nucleic acid molecules, or the above expression cassette; or
(b.1) introducing of a site-directed nuclease and a repair matrix into at least one cell of a maize plant, wherein the site-directed nuclease is able to generate at least one double-strand break of the DNA in the genome of the at least one cell and the repair matrix comprises the above nucleic acid molecules or a fragment thereof; or
(b.2) cultivation of the at least one cell of (b.1) under conditions that allow a homology-directed repair or a homologous recombination, wherein the nucleic acid molecule is integrated from the repair matrix into the genome of the maize plant; and
(c) obtaining the plant having anthracnose stalk rot resistance from the at least one cell.

In some embodiments, the site-directed nuclease comprises a zinc-finger nuclease, a transcription activator-like effector nuclease, a CRISPR/Cas system, including a CRISPR/Cas9 system, a CRISPR/Cpf1 system, a CRISPR/MAD7, a CRISPR/CasX system, a CRISPR/CasY system, a Prime Editing system, a CRISPR-based base editor system, an engineered homing endonuclease, and a meganuclease, and/or any combination, variant, or catalytically active fragment thereof.

Also provided is a maize plant identified according to any of the above processes, or manufactured according to the above method for manufacturing a maize plant having anthracnose stalk rot resistance.

In another aspect is provided a maize plant comprising a resistance locus associated with anthracnose stalk rot resistance, wherein the maize plant is prepared by any of the above-described processes comprising introgressing the resistance locus into the maize plant.

In another aspect is provided a maize plant comprising a resistance locus associated with anthracnose stalk rot resistance, wherein the maize plant is prepared by a process comprising introgressing the above nucleic acid molecules into the maize plant.

In another aspect is provided a maize plant comprising a resistance locus associated with anthracnose stalk rot resistance, wherein the maize plant is prepared by a process comprising introducing a nucleic acid into the maize plant, wherein the nucleic acid comprises
i. a nucleotide sequence of SEQ ID NO: 266 or 269,
ii. a nucleotide sequence having coding sequence of SEQ ID NO: 267 or 270,
iii. a nucleotide sequence which is complementary to a sequence from i. or ii.,
iv. a nucleotide sequence which has at least 80% or 85%, preferably at least 90%, 91%, 92%, 93%, 94% or 95%, more preferably at least 96%, 97%, 98% or 99% identity with a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein with one of the SEQ ID NO: 268 or 271,
viii. a nucleotide sequence which encodes for a protein which has at least 80% or 850%, preferably at least 90%, 91%, 92%, 93%, 94% or 95%, more preferably at least 96%, 97%, 98% or 99% identity with a sequence of SEQ ID NO: 213 or 216,
ix. a nucleotide sequence comprising or consisting of a nucleotide sequence of SEQ ID NO: 272, or a fragment thereof,
x. a nucleic acid molecule encoding anthracnose stalk rot resistance allele having a haplotype comprising on or more polymorphisms selected recited in Table 13, or
xi. a nucleic acid molecule encoding a anthracnose stalk rot resistance allele having a haplotype comprising one or more nucleotide polymorphisms selected from the group consisting of:
   a “C” at position 413 referenced to SEQ ID NO: 50,
   a “C” at position 958 referenced to SEQ ID NO: 50,
   a “C” at position 971 referenced to SEQ ID NO: 50,
   a “T” at position 1099 referenced to SEQ ID NO: 50,
   an “A” at position 1154 referenced to SEQ ID NO: 50,
   a “T” at position 1250 referenced to SEQ ID NO: 50,
   a “G” at position 1607 referenced to SEQ ID NO: 50,
   a “G” at position 2001 referenced to SEQ ID NO: 50,
   an “A” at position 2598 referenced to SEQ ID NO: 50, and
   an “A” at position 3342 referenced to SEQ ID NO: 50.

In various embodiments, the nucleic acid molecule encodes a polypeptide capable of conferring or increasing resistance to a plant disease caused by fungal pathogen in a plant in which the polypeptide is expressed.

In another aspect a maize plant is provided, wherein the maize plant is selected according to a method comprising:

a. obtaining a first maize plant that comprises within its genome a haplotype comprising one or more of
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50,
x. an “A” at position 3342 referenced to SEQ ID NO: 50, and

### xi. a “G” at C16759-001-K1; and

b. crossing the first maize plant to a second maize plant;
c. evaluating progeny plants for the haplotype in a. or at least one marker allele linked to and associated with the haplotype in b.; and
d. selecting progeny plants that possess the haplotype in a.
In some embodiments, the maize plant comprises within its genome a haplotype comprising a “G” at C16759-001-K1 and one or more of
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50, and
x. an “A” at position 3342 referenced to SEQ ID NO: 50.

Also provided is a seed or plant part of any of the above maize plants.

### DETAILED DESCRIPTION

From identification of two novel genetic resources of corn on chromosome 4 and 6, provided are materials and molecular genetic selection methods for enhancing resistance to plant fungal pathogens, in particular causative pathogen of anthracnose stalk rot. Resistance loci on chromosome 4 and chromosome 6 are described, such as in the Examples. Both resistance loci on chromosome 4 and chromosome 6 can be used together for breeding a corn plant. Alternatively, only the locus on chromosome 4 can be used for breeding a corn plant. Also, only the locus on chromosome 6 can be used for breeding a corn plant. Thus, the loci can be used together, or as separated loci, i.e., as individual traits. Breeders can use information provided in the Examples and throughout the disclosure to track the resistance loci in the breeding material by standardized marker technology like KASP. The genetic characterization provided in the Examples and throughout the disclosure can allow for the clear distinction of the two loci from other known ASR resistance loci on these chromosomes.

### Definitions

Unless defined otherwise, technical and scientific terms used herein have the same meaning as commonly understood by one of ordinary skill in the art to which this disclosure belongs.

As used herein, the term “plant” can be a whole plant, any part thereof, or a cell or tissue culture derived from a plant. Thus, the term “plant” can refer to any of. whole plants, plant components or organs (including but not limited to embryos, pollen, ovules, seeds, leaves, flowers, branches, fruit, kernels, ears, cobs, husks, stalks, roots, root tips, anthers, and the like), plant tissues, plant cells, plant protoplasts, plant cell tissue cultures from which maize plant can be regenerated, plant calli, plant clumps, and plant seeds. A plant cell is a cell of a plant, either taken directly from a seed or plant, or derived through culture from a cell taken from a plant. Grain is intended to mean the mature seed produced by commercial growers for purposes other than growing or reproducing the species. Progeny, variants, and mutants of the regenerated plants are also included within the scope of the embodiments, provided that these parts comprise the introduced polynucleotides.

The term “locus” generally refers to a genetically defined region of a chromosome carrying a gene or, possibly, two or more genes so closely linked that genetically they behave as a single locus responsible for a phenotype. A “gene” shall refer to a specific genetic coding region within a locus, including its associated regulatory sequences.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

The term “allele” refers to one of two or more different nucleotide sequences that occur at a specific locus. A first allele is found on one chromosome, while a second allele occurs at the same position on the homologue of that chromosome, e.g., as occurs for different chromosomes of a heterozygous individual, or between different homozygous or heterozygous individuals in a population. “Allele frequency” refers to the frequency (proportion or percentage) of an allele within a population, or a population of lines. One can estimate the allele frequency within a population by averaging the allele frequencies of a sample of individuals from that population.

An allele “positively” correlates with a trait when it is linked to it and when presence of the allele is an indicator that the desired trait or trait form will occur in a plant comprising the allele. An allele negatively correlates with a trait when it is linked to it and when presence of the allele is an indicator that a desired trait or trait form will not occur in a plant comprising the allele.

The terms “amplify” and “amplifying” in the context of nucleic acid amplification refer to any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “molecular marker” can refer to a genetic marker, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term “molecular marker” can also refer to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, a marker probe refers to a probe of any type that is able to distinguish (i.e., genotype) the particular allele that is present at a marker locus.

Nucleic acids are “complementary” when they specifically hybridize in solution, e.g., according to Watson-Crick base pairing rules. Some of the markers described herein are also referred to as hybridization markers when located on an indel region, such as the non-colinear region described herein. This is because the insertion region is, by definition, a polymorphism vis-à-vis a plant without the insertion. Thus, the marker need only indicate whether the indel region is present or absent. Any suitable marker detection technology may be used to identify such a hybridization marker, e.g. SNP technology is used in the examples provided herein.

As used herein, “linked” or “linkage” (as distinguished from the term “operably linked”) shall refer to the genetic or physical linkage of loci or genes. Loci or genes are considered genetically linked if the recombination frequency between them is less than about 50% as determined on a single meiosis map. They are progressively more linked if the recombination frequency is about 40%, about 30%, about 20%, about 10% or less, as determined on a single meiosis map. Two or more genes are physically linked (or syntenic) if they have been demonstrated to be on a single piece of DNA, such as a chromosome. Genetically linked genes will in practice be physically linked (or syntenic), but the exact physical distance (number of nucleotides) may not have been demonstrated yet.

As used herein, “introgression” or “introgressing” shall refer to moving a gene or locus from one line to another by: (1) crossing individuals of each line to create a population; and (2) selecting individuals carrying the desired gene or locus. Selection may be done phenotypically or using markers (marker assisted selection). The individuals so selected are again crossed (i.e., backcrossed) with the desired target line; there may be two, three, four, five, six or more, or even ten or more backcrosses. After each cross, the selection process is repeated. For example, the gene of the embodiments, or the locus containing it, may be introgressed into a recurrent parent that is not resistant or only partially resistant, meaning that it is sensitive or susceptible or partially so, to Cg (Colletotrichum graminicola). The recurrent parent line with the introgressed gene or locus then has enhanced or newly conferred resistance to Cg. This line into which the anthracnose stalk rot resistance locus has been introgressed is referred to herein as an anthracnose stalk rot resistance locus conversion.

The process of introgressing is often referred to as “backcrossing” when the process is repeated two or more times. In introgressing or backcrossing, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al., “Marker-assisted backcrossing: a practical example” in Techniques et Utilisations des Marqueurs Moleculaires Les Colloques, 1995, Vol. 72, pp. 45-56 and Openshaw et al., “Marker-assisted Selection in Backcross Breeding, Analysis of Molecular Marker Data”, 1994, pages 41-43. The initial cross gives rise to the F1 generation; the term “BC1” then refers to the second use of the recurrent parent, “BC2” refers to the third use of the recurrent parent, and so on.

The terms “pathogen resistance”, “fungal resistance”, and “disease resistance” are intended to mean that the plant avoids the disease symptoms that are the outcome of plant-pathogen interactions. That is, pathogens are prevented from causing plant diseases and the associated disease symptoms, or alternatively, the disease symptoms caused by the pathogen are minimized or lessened, such as, for example, the reduction of stress and associated yield loss. One of skill in the art will appreciate that the compositions and methods disclosed herein can be used with other compositions and methods available in the art for protecting plants from pathogen attack.

As used herein, “fungal resistance” refers to enhanced resistance or tolerance to a fungal pathogen when compared to that of a wild type plant. Effects may vary from a slight increase in tolerance to the effects of the fungal pathogen (e.g., partial inhibition) to total resistance such that the plant is unaffected by the presence of the fungal pathogen. An increased level of resistance against a particular fungal pathogen or against a wider spectrum of fungal pathogens constitutes “enhanced” or improved fungal resistance. The embodiments of the disclosure also will enhance or improve fungal plant pathogen resistance, such that the resistance of the plant to a fungal pathogen or pathogens will increase. The term “enhance” refers to improve, increase, amplify, multiply, elevate, raise, and the like. Herein, plants of the disclosure are described as being resistant to infection by Cg or having ‘enhanced resistance’ to infection by Cg as a result of the loci at chromosomes 4 and 6 described herein.

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

“Transformation” refers to the transfer of a nucleic acid fragment into the genome of a host organism, resulting in genetically stable inheritance. Host organisms containing the transformed nucleic acid fragments are referred to as “transgenic” organisms. “Host cell” refers the cell into which transformation of the recombinant DNA construct takes place and may include a yeast cell, a bacterial cell, and a plant cell. Examples of methods of plant transformation include Agrobacterium-mediated transformation and particle-bombardment technology.

“Stable transformation” is intended to mean that the nucleotide construct introduced into a plant integrates into the genome of the plant and is capable of being inherited by the progeny thereof. “Transient transformation” or “transient expression” is intended to mean that a polynucleotide is introduced into the plant and does not integrate into the genome of the plant or a polypeptide is introduced into a plant.

As used herein, “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides can be produced either from a nucleic acid disclosed herein, or by the use of standard molecular biology techniques. For example, a truncated protein can be produced by expression of a recombinant nucleic acid in an appropriate host cell, or alternatively by a combination of ex vivo procedures, such as protease digestion and purification.

As used herein, “full-length sequence,” in reference to a specified polynucleotide, means having the entire nucleic acid sequence of a native sequence. “Native sequence” is intended to mean an endogenous sequence, i.e., a non-engineered sequence found in an organism's genome.

A “fragment” is a portion of the nucleotide sequence or a portion of the amino acid sequence and hence protein encoded thereby. Fragments of a nucleotide sequence may encode protein fragments that retain the biological activity of the native protein and hence have the ability to confer fungal resistance upon a plant. Alternatively, fragments of a nucleotide sequence that are useful as hybridization probes do not necessarily encode fragment proteins retaining biological activity. Thus, fragments of a nucleotide sequence may range from at least about 15 nucleotides, about 50 nucleotides, about 100 nucleotides, and up to the full-length nucleotide sequence encoding the polypeptides of the embodiments.

The term “variant” is intended to mean a substantially similar sequence. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. Variants of the nucleic acids of the embodiments can be constructed such that the open reading frame is maintained. For polynucleotides, conservative variants include those sequences that, because of the degeneracy of the genetic code, encode the amino acid sequence of one of the polypeptides of the embodiments. Variant polynucleotides also include synthetically derived polynucleotides, such as those generated, for example, by using site-directed mutagenesis but which still encode a protein of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least about 20 contiguous nucleotides in length, and optionally can be about 30, about 40, about 50, about 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm.

The nucleic acids, polypeptides, and markers of the embodiments find use in methods for conferring or enhancing fungal resistance to a plant. Accordingly, the compositions and methods disclosed herein are useful for identifying plants resistant to fungal pathogens, such as pathogens causing anthracnose stalk rot.

### Variants

The genes and polynucleotides described herein may be comprised in naturally occurring sequences as well as mutant forms. Likewise, the proteins described herein may encompass both naturally occurring proteins as well as variations and modified forms thereof. Such variants can possess the desired ability to confer or enhance plant fungal pathogen resistance.

Variant polynucleotides and proteins may also be comprised of sequences and proteins derived from mutagenic or recombinogenic procedures, including and not limited to procedures such as DNA shuffling. One of skill in the art could envision modifications that would alter the range of pathogens to which the protein responds. With such a procedure, one or more different protein coding sequences can be manipulated to create a new protein possessing the desired properties. In this manner, libraries of recombinant polynucleotides are generated from a population of related sequence polynucleotides comprising sequence regions that have substantial sequence identity and can be homologously recombined in vitro or in vivo. For example, sequence motifs encoding a domain of interest may be shuffled between a resistance gene described herein and other known genes to obtain a new gene coding for a protein with an improved property of interest, such as increased ability to confer or enhance plant fungal pathogen resistance. Strategies for such DNA shuffling are known in the art. See, for example, Stemmer, Proc. Natl. Acad. Sci., 1994, USA 91:10747-10751; Stemmer, Nature, 1994, 370:389-391; Crameri et al., Nature Biotech., 1997, 15:436-438; Moore et al., J. Mol. Biol., 1997, 272:336-347; Zhang et al., Proc. Natl. Acad. Sci. USA, 1997, 94:4504-4509; Crameri et al., Nature, 1998, 391:288-291; and U.S. Pat. Nos. 5,605,793 and 5,837,458.

For example, an entire polynucleotide disclosed herein, or one or more portions thereof, may be used as a probe capable of specifically hybridizing to corresponding polynucleotides and messenger RNAs. To achieve specific hybridization under a variety of conditions, such probes include sequences that are unique and are optimally at least about 10 nucleotides in length, at least about 15 nucleotides in length, or at least about 20 nucleotides in length. Such probes may be used to amplify corresponding polynucleotides from a chosen organism by PCR. This technique may be used to isolate additional coding sequences from a desired organism or as a diagnostic assay to determine the presence of coding sequences in an organism. Hybridization techniques include hybridization screening of plated DNA libraries.

Hybridization of such sequences may be carried out under stringent conditions. “Stringent conditions” or “stringent hybridization conditions” are conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified by homologous probing. Alternatively, stringency conditions can be adjusted to allow some mismatching in sequences so that lower degrees of similarity are detected by heterologous probing. Generally, a probe is less than about 1000 nucleotides in length, optimally less than 500 nucleotides in length.

The proteins described herein may be altered in various ways including amino acid substitutions, deletions, truncations, and insertions. Methods for such manipulations are generally known in the art. For example, amino acid sequence variants and fragments of anti-pathogenic proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

### Molecular Markers

Molecular markers can be used in a variety of plant breeding applications (see Staub et al. Hortscience, 1996, 31: 729-741; and Tanksley, Plant Molecular Biology Reporter, 1993, 1: 3-8). For detecting recombinations, markers need to detect differences, or polymorphisms, within the population being monitored. Differences at the DNA level due to polynucleotide sequence differences (e.g., SSRs, RFLPs, FLPs, SNPs) are detected by molecular markers. The genomic variability can be of any origin, for example, insertions, deletions, duplications, repetitive elements, point mutations, recombination events, or the presence and sequence of transposable elements. Molecular markers can be derived from genomic or expressed nucleic acids (e.g., ESTs). ESTs are generally well conserved within a species, while other regions of DNA (typically non-coding) tend to accumulate polymorphism, and therefore, can be more variable between individuals of the same species. A large number of corn molecular markers are known in the art, and are published or available from various sources, such as the Maize GDB and the Arizona Genomics Institute.

Marker-assisted selection may be used to increase the efficiency of backcrossing and introgressing genes. A molecular marker that demonstrates linkage with a locus affecting a desired phenotypic trait may provide a useful tool for the selection of the trait in a plant population. This is particularly true where the phenotype is hard to assay, e.g., many disease resistance traits, or, occurs at a late stage in plant development, e.g., kernel characteristics. Since DNA marker assays are less laborious, and take up less physical space, than field phenotyping, much larger populations can be assayed to increase the chances of finding a recombinant with the target segment from the donor line moved to the recipient line. The closer the linkage, the more useful the marker because recombination is less likely to occur between the marker and the gene causing the trait. The reduced rate of recombination can result in fewer false positives. Having flanking markers decreases the chances that false positive selection will occur as a double recombination event would be needed.

Various types of fragment length polymorphisms or FLP markers can be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al., Plant Mol Biol 2002, 48, 539-547). The term “indel” refers to an insertion or deletion, wherein one line may be referred to as having an insertion relative to a second line, or the second line may be referred to as having a deletion relative to the first line. The MZA markers disclosed herein are examples of amplified FLP markers that have been selected because they are in close proximity to the Rcg1 and Rcg1b genes.

SNP markers detect single base pair nucleotide substitutions. Of all the molecular marker types, SNPs are the most abundant, thus having the potential to provide the highest genetic map resolution (Bhattramakki et al., 2002 Plant Molecular Biology 48:539-547). SNPs can be assayed at an even higher level of throughput than SSRs, such as in an ultra-high-throughput fashion. Several methods are available for SNP genotyping, including but not limited to, hybridization, primer extension, oligonucleotide ligation, nuclease cleavage, mini-sequencing and coded spheres.

A number of SNPs together within a sequence, or across linked sequences, can be used to describe a haplotype for any particular genotype. Haplotypes can be more informative than single SNPs and can be more descriptive of any particular genotype. For example, a single SNP may be allele ‘T’ for MP305, but the allele ‘T’ might also occur in the maize breeding population being utilized for recurrent parents. In this case, a haplotype, e.g. a series of alleles at linked SNP markers, may be more informative. Once a unique haplotype has been assigned to a donor chromosomal region, that haplotype can be used in that population or any subset thereof to determine whether an individual has a particular gene. Using automated high throughput marker detection platforms may make this process highly efficient and effective.

Various primers described herein can be used as FLP markers to select for the anthracnose stalk rot resistance locus on chromosome 4 or 6 of Zea mays. Exemplary primers include, but are not limited to, those of SEQ ID NOS: 95-118 and 120-125. These primers can also be used to convert these markers to SNP or other structurally similar or functionally equivalent markers (e.g., SSRs, CAPs, and indels), in the same regions. Using PCR, the primers can be used to amplify DNA segments from individuals (preferably inbred) that represent the diversity in the population of interest. The PCR products can be sequenced directly in one or both directions. The resulting sequences may then aligned and polymorphisms identified. The polymorphisms are not limited to single nucleotide polymorphisms (SNPs), but also include indels, CAPS, SSRs, and VNTRs (variable number of tandem repeats). Specifically with respect to the fine map information described herein, one can readily use the information provided herein to obtain additional polymorphic SNPs (and other markers) within the region amplified by the primers listed in this disclosure. Markers within the described map region can be hybridized to BACs or other genomic libraries, or electronically aligned with genome sequences, to find new sequences in the same approximate location as the described markers.

### Nucleic Acids

In one aspect is provided a nucleic acid comprising the sequence of SEQ ID NO: 209, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 209. In one aspect is provided a nucleic acid comprising the sequence of SEQ ID NO: 212, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 212. In one aspect is provided a nucleic acid comprising the sequence of SEQ ID NO: 215, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 215. In one aspect is provided a nucleic acid comprising the sequence of SEQ ID NO: 218, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 218. In one aspect is provided a nucleic acid comprising the sequence of SEQ ID NO: 221, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 221. In one aspect is provided a nucleic acid comprising the sequence of SEQ ID NO: 224, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 224. In one aspect is provided a nucleic acid comprising the sequence of SEQ ID NO: 227, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 227. In one aspect is provided a nucleic acid comprising the sequence of SEQ ID NO: 230, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 230. In one aspect is provided a nucleic acid comprising the sequence of SEQ ID NO: 233, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 233. In one aspect is provided a nucleic acid comprising the sequence of SEQ ID NO: 236, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 236. In one aspect is provided a nucleic acid comprising the sequence of SEQ ID NO: 239, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 239. In one aspect is provided a nucleic acid comprising the sequence of SEQ ID NO: 242, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 242. In one aspect is provided a nucleic acid comprising the sequence of SEQ ID NO: 245, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 245. In one aspect is provided a nucleic acid comprising the sequence of SEQ ID NO: 248, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 248. In one aspect is provided a nucleic acid comprising the sequence of SEQ ID NO: 251, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 251. In one aspect is provided a nucleic acid comprising the sequence of SEQ ID NO: 254, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 254. In one aspect is provided a nucleic acid comprising the sequence of SEQ ID NO: 257, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 257. In one aspect is provided a nucleic acid comprising the sequence of SEQ ID NO: 260, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 260. In one aspect is provided a nucleic acid comprising the sequence of SEQ ID NO: 263, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 263. In one aspect is provided a nucleic acid comprising the sequence of SEQ ID NO: 266, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 266. In one aspect is provided a nucleic acid comprising the sequence of SEQ ID NO: 267, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 267. In one aspect is provided a nucleic acid comprising the sequence of SEQ ID NO: 269, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 269. In one aspect is provided a nucleic acid comprising the sequence of SEQ ID NO: 270, and a sequence at least 80, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 270. In one aspect is provided a nucleic acid comprising the sequence of SEQ ID NO: 272, or a fragment thereof, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 272, or a fragment thereof.

In one aspect is provided a nucleic acid comprising the sequence of SEQ ID NO: 1, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 1. In one aspect is provided a nucleic acid comprising the sequence of SEQ ID NO: 20, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 20. In one aspect is provided a nucleic acid comprising the sequence of SEQ ID NO: 23, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 23. In one aspect is provided a nucleic acid comprising the sequence of SEQ ID NO: 44, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 44. In one aspect is provided a nucleic acid comprising the sequence of SEQ ID NO: 47, and a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 47. Any of these nucleic acids may comprise additional sequence from a resistance locus on chromosome 6 of Zea mays. Also provided are maize plants comprising the nucleotide sequence. In various embodiments, the maize plant is more resistant to stalk rot than a comparable maize plant that does not comprise the nucleic acid.

In one aspect is provided a nucleic acid comprising a sequence complementary to SEQ ID NO: 209, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 209. In one aspect is provided a nucleic acid comprising a sequence complementary to SEQ ID NO: 212, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 212. In one aspect is provided a nucleic acid comprising a sequence complementary to SEQ ID NO: 215, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 215. In one aspect is provided a nucleic acid comprising a sequence complementary to SEQ ID NO: 218, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 218. In one aspect is provided a nucleic acid comprising a sequence complementary to SEQ ID NO: 221, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 221. In one aspect is provided a nucleic acid comprising a sequence complementary to SEQ ID NO: 224, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 224. In one aspect is provided a nucleic acid comprising a sequence complementary to SEQ ID NO: 227, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 227. In one aspect is provided a nucleic acid comprising a sequence complementary to SEQ ID NO: 230, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 230. In one aspect is provided a nucleic acid comprising a sequence complementary to SEQ ID NO: 233, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 233. In one aspect is provided a nucleic acid comprising a sequence complementary to SEQ ID NO: 236, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 236. In one aspect is provided a nucleic acid comprising a sequence complementary to SEQ ID NO: 239, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 239. In one aspect is provided a nucleic acid comprising a sequence complementary to SEQ ID NO: 242, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 242. In one aspect is provided a nucleic acid comprising a sequence complementary to SEQ ID NO: 245, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 245. In one aspect is provided a nucleic acid comprising a sequence complementary to SEQ ID NO: 248, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 248. In one aspect is provided a nucleic acid comprising a sequence complementary to SEQ ID NO: 251, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 251. In one aspect is provided a nucleic acid comprising a sequence complementary to SEQ ID NO: 254, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 254. In one aspect is provided a nucleic acid comprising a sequence complementary to SEQ ID NO: 257, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 257. In one aspect is provided a nucleic acid comprising a sequence complementary to SEQ ID NO: 260, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 260. In one aspect is provided a nucleic acid comprising a sequence complementary to SEQ ID NO: 263, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 263. In one aspect is provided a nucleic acid comprising a sequence complementary to SEQ ID NO: 266, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 266. In one aspect is provided a nucleic acid comprising a sequence complementary to SEQ ID NO: 267, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 267. In one aspect is provided a nucleic acid comprising a sequence complementary to SEQ ID NO: 269, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 269. In one aspect is provided a nucleic acid comprising a sequence complementary to SEQ ID NO: 270, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 270. In one aspect is provided a nucleic acid comprising a sequence complementary to SEQ ID NO: 272, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 272. In one aspect is provided a nucleic acid comprising a sequence complementary to SEQ ID NO: 1, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 1. In one aspect is provided a nucleic acid comprising a sequence complementary to SEQ ID NO: 20, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 20. In one aspect is provided a nucleic acid comprising a sequence complementary to SEQ ID NO: 23, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 23. In one aspect is provided a nucleic acid comprising a sequence complementary to SEQ ID NO: 44, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 44. In one aspect is provided a nucleic acid comprising a sequence complementary to SEQ ID NO: 47, or a sequence complementary to a sequence at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO: 47. Any of these nucleic acids may comprise additional sequence from a resistance locus on chromosome 6 of Zea mays. Also provided are maize plants comprising the nucleotide sequence. In various embodiments, the maize plant is more resistant to stalk rot than a comparable maize plant that does not comprise the nucleic acid. In some embodiments, the stalk rot is anthracnose stalk rot. In some embodiments, the stalk rot is Fusarium stalk rot.

Also provided are nucleic acids that hybridize to any of the above nucleic acids under stringent conditions.

In another aspect is provided a nucleic acid comprising a nucleotide sequence encoding for a protein comprising the sequence of any one of SEQ ID NO: 211, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of any one of SEQ ID NOS: 211. Also provided is a nucleic acid comprising a nucleotide sequence encoding for a protein comprising the sequence of SEQ ID NO: 214, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 214. Also provided is a nucleic acid comprising a nucleotide sequence encoding for a protein comprising the sequence of SEQ ID NO: 217, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 217. Also provided is a nucleic acid comprising a nucleotide sequence encoding for a protein comprising the sequence of SEQ ID NO: 220, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 220. Also provided is a nucleic acid comprising a nucleotide sequence encoding for a protein comprising the sequence of SEQ ID NO: 223, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 223. Also provided is a nucleic acid comprising a nucleotide sequence encoding for a protein comprising the sequence of SEQ ID NO: 226, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 226. Also provided is a nucleic acid comprising a nucleotide sequence encoding for a protein comprising the sequence of SEQ ID NO: 229, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 229. Also provided is a nucleic acid comprising a nucleotide sequence encoding for a protein comprising the sequence of SEQ ID NO: 232, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 232. Also provided is a nucleic acid comprising a nucleotide sequence encoding for a protein comprising the sequence of SEQ ID NO: 235, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 235. Also provided is a nucleic acid comprising a nucleotide sequence encoding for a protein comprising the sequence of SEQ ID NO: 238, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 238. Also provided is a nucleic acid comprising a nucleotide sequence encoding for a protein comprising the sequence of SEQ ID NO: 241, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 241. Also provided is a nucleic acid comprising a nucleotide sequence encoding for a protein comprising the sequence of SEQ ID NO: 244, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 244. Also provided is a nucleic acid comprising a nucleotide sequence encoding for a protein comprising the sequence of SEQ ID NO: 247, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 247. Also provided is a nucleic acid comprising a nucleotide sequence encoding for a protein comprising the sequence of SEQ ID NO: 250, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 250. Also provided is a nucleic acid comprising a nucleotide sequence encoding for a protein comprising the sequence of SEQ ID NO: 253, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 253. Also provided is a nucleic acid comprising a nucleotide sequence encoding for a protein comprising the sequence of SEQ ID NO: 256, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 256. Also provided is a nucleic acid comprising a nucleotide sequence encoding for a protein comprising the sequence of SEQ ID NO: 259, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 259. Also provided is a nucleic acid comprising a nucleotide sequence encoding for a protein comprising the sequence of SEQ ID NO: 262, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 262. Also provided is a nucleic acid comprising a nucleotide sequence encoding for a protein comprising the sequence of SEQ ID NO: 265, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 265. Also provided is a nucleic acid comprising a nucleotide sequence encoding for a protein comprising the sequence of SEQ ID NO: 268, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 268. Also provided is a nucleic acid comprising a nucleotide sequence encoding for a protein comprising the sequence of SEQ ID NO: 271, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 271.

In another aspect is provided a nucleic acid comprising a nucleotide sequence encoding for a protein comprising the sequence of any one of SEQ ID NOs: 11-19, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of any one of SEQ ID NOS: 11-19. Also provided is a nucleic acid comprising a nucleotide sequence encoding for a protein comprising the sequence of SEQ ID NO: 22, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 22. Also provided is a nucleic acid comprising a nucleotide sequence encoding for a protein comprising the sequence of any one of SEQ ID NOS: 34-43, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of any one of SEQ ID NOS: 34-43.

Also provided is a nucleic acid comprising a nucleotide sequence encoding for a protein comprising the sequence of SEQ ID NO: 46, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 46. Also provided is a nucleic acid comprising a nucleotide sequence encoding for a protein comprising the sequence of SEQ ID NO: 49, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 49.

In another aspect is provided a nucleic acid molecule encoding a Rcg1 resistance allele having a haplotype comprising one or more single nucleotide polymorphism selected from the group consisting of:
a. a “C” at position 413 referenced to SEQ ID NO: 50,
b. a “C” at position 958 referenced to SEQ ID NO: 50,
c. a “C” at position 971 referenced to SEQ ID NO: 50,
d. a “T” at position 1099 referenced to SEQ ID NO: 50,
e. an “A” at position 1154 referenced to SEQ ID NO: 50,
f. a “T” at position 1250 referenced to SEQ ID NO: 50,
g. a “G” at position 1607 referenced to SEQ ID NO: 50,
h. a “G” at position 2001 referenced to SEQ ID NO: 50,
i. an “A” at position 2598 referenced to SEQ ID NO: 50, and
j. an “A” at position 3342 referenced to SEQ ID NO: 50, where the nucleic acid molecule is encoding a polypeptide capable of conferring or increasing resistance to a plant disease caused by fungal pathogen in a plant in which the polypeptide is expressed.

The resistance allele is found on chromosome 4 of Zea mays. In various embodiments, the nucleic acid molecule does not produce the amplicon (e.g., amplicon 7) according to SEQ ID NO: 119 upon polymerase chain reaction amplification by means of primers of SEQ ID NO: 120 and 121, primers of SEQ ID NO: 122 and 123, or primers of SEQ ID NO: 124 and 125. In various embodiments, the nucleic acid molecule does not produce an amplicon selected from the group consisting of SEQ ID NOs: 94, 101, 106, 109, and 114 (e.g., amplicons 2-6) upon polymerase chain reaction amplification by means of primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118.

Also provided are isolated or substantially purified polynucleotide compositions comprising one or more of the above nucleic acid molecules. An “isolated” or “purified” polynucleotide, or biologically active portion thereof, is substantially or essentially free from components that normally accompany or interact with the polynucleotide or protein as found in its naturally occurring environment. Thus, an isolated or purified polynucleotide is substantially free of other cellular material, or culture medium when produced by recombinant techniques (e.g. PCR amplification), or substantially free of chemical precursors or other chemicals when chemically synthesized. Optimally, an “isolated” polynucleotide is free of sequences (for example, protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, about 4 kb, about 3 kb, about 2 kb, about 1 kb, about 0.5 kb, or about 0.1 kb of nucleotide sequence that naturally flank the polynucleotide in genomic DNA of the cell from which the polynucleotide is derived.

### Expression Cassettes

The terms “recombinant construct,” “expression cassette,” “expression construct,” “chimeric construct,” “construct,” “recombinant DNA construct” and “recombinant DNA fragment” are used interchangeably herein and are nucleic acid fragments or refer to a construct assembled from nucleic acid fragments, which may be obtained from different sources or organisms. A recombinant construct comprises an artificial combination of nucleic acid fragments, including, and not limited to, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature or that may be combined according to the needs of a person skilled in the art. Such construct may be used by itself or may be used in conjunction with a vector. If a vector is used, then the choice of vector is dependent upon the method that will be used to transform host cells as is well known to those skilled in the art. Suitable vectors may be a plasmid vector, a viral vector, a cosmid, bacmid or artificial chromosome. Further, non-limiting examples may be the Ti-plasmid of Agrobacterium tumefaciens or the viral DNA vector derived from plant viruses. The skilled artisan is well aware of the genetic elements that must be present on the vector in order to successfully transform, select and propagate host cells comprising any of the isolated nucleic acid fragments of the embodiments. Screening to obtain lines displaying the desired expression level and pattern of the polynucleotides or of the anthracnose stalk rot resistance locus may be accomplished by amplification, Southern analysis of DNA, northern blot analysis of mRNA expression, immunoblotting analysis of protein expression, phenotypic analysis, and the like.

In various aspects are provided expression cassettes comprising any of the nucleic acids and polynucleotides described herein. The nucleic acid or polynucleotide may comprise sequence from the resistance locus on chromosome 4. The nucleic acid or polynucleotide may comprise sequence from the resistance locus on chromosome 6. The nucleic acid or polynucleotide may comprise one or more sequences from both the resistance locus on chromosome 4 and the resistance locus on chromosome 6.

In some embodiments, expression cassettes comprising a promoter operably linked to a heterologous nucleotide sequence of the embodiments are further provided. The expression cassettes of the embodiments find use in generating transformed plants, plant cells, and microorganisms and in practicing the methods for inducing plant fungal pathogen resistance disclosed herein. The expression cassette will include 5′ and 3′ regulatory sequences operably linked to a polynucleotide of the embodiments. “Operably linked” is intended to mean a functional linkage between two or more elements. “Regulatory sequences” refer to nucleotides located upstream (5′ non-coding sequences), within, or downstream (3′ non-coding sequences) of a coding sequence, and which may influence the transcription, RNA processing, stability, or translation of the associated coding sequence. Regulatory sequences may include, and are not limited to, promoters, translation leader sequences, introns, and polyadenylation recognition sequences. For example, an operable linkage between a polynucleotide of interest and a regulatory sequence (a promoter, for example) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be co-transformed into the organism. Alternatively, the additional gene(s) can be provided on multiple expression cassettes. Such an expression cassette is provided with a plurality of restriction sites and/or recombination sites for insertion of the polynucleotide that encodes an antipathogenic polypeptide to be under the transcriptional regulation of the regulatory regions. The expression cassette may additionally contain selectable marker genes.

The expression cassette can include in the 5′-3′ direction of transcription, a transcriptional initiation region (i.e., a promoter), translational initiation region, a polynucleotide of the embodiments, a translational termination region and, optionally, a transcriptional termination region functional in the host organism. The regulatory regions (i.e., promoters, transcriptional regulatory regions, and translational termination regions) and/or the polynucleotide of the embodiments may be native/analogous to the host cell or to each other. Alternatively, the regulatory regions and/or the polynucleotide of the embodiments may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The optionally included termination region may be native with the transcriptional initiation region, may be native with the operably linked polynucleotide of interest, may be native with the plant host, or may be derived from another source (i.e., foreign or heterologous) to the promoter, the polynucleotide of interest, the host, or any combination thereof. Convenient termination regions are available from the Ti-plasmid of A. tumefaciens, such as the octopine synthase and nopaline synthase termination regions. In particular embodiments, the potato protease inhibitor II gene (PinII) terminator is used. See, for example, Keil et al., Nucl. Acids Res., 1986, 14:5641-5650; and An et al., Plant Cell, 1989, 1:115-122, herein incorporated by reference in their entirety.

A number of promoters can be used in the practice of the embodiments, including the native promoter of the polynucleotide sequence of interest. The promoters can be selected based on the desired outcome. A wide range of plant promoters are discussed in the recent review of Potenza et al., In Vitro Cell Dev Biol-Plant, 2004, 40:1-22, herein incorporated by reference. For example, the nucleic acids can be combined with constitutive, tissue-preferred, pathogen-inducible, or other promoters for expression in plants. Such constitutive promoters include, for example, the core promoter of the Rsyn7 promoter.

It may be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See WO 99/43819, which is incorporated by reference herein in its entirety. Also of interest are promoters that result in expression of a protein locally at or near the site of pathogen infection. Of particular interest is the inducible promoter for the maize PRms gene, whose expression is induced by the pathogen Fusarium moniliforme (see, for example, Cordero et al. (1992) Physiol. Mol. Plant. Path. 41:189-200).

Additionally, as pathogens find entry into plants through wounds or insect damage, a wound-inducible promoter may be used in the constructions of the embodiments. Exemplary wound-inducible promoters include, but are not limited to, potato proteinase inhibitor (pin II) gene, wun1, wun2, win1, win2, WIP1, and MPI.

Chemical-regulated promoters can be used to modulate the expression of a gene in a plant through the application of an exogenous chemical regulator or agent. Depending upon the objective, the promoter may be a chemical-inducible promoter, where application of the chemical induces gene expression, or a chemical-repressible promoter, where application of the chemical represses gene expression. Chemical-inducible promoters are known in the art and include, and are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters and tetracycline-inducible and tetracycline-repressible promoters.

Tissue-specific promoters can be utilized to target enhanced expression of the polypeptides of the embodiments within a particular plant tissue. For example, a tissue-specific promoter may be used to express a polypeptide in a plant tissue where disease resistance is particularly important, such as, for example, the roots, the stalk or the leaves. Such promoters can be modified, if necessary, for weak expression. Vascular tissue-preferred promoters are known in the art and include those promoters that selectively drive protein expression in, for example, xylem and phloem tissue. Stalk-preferred promoters may be used to drive expression of a polypeptide of the embodiments. Exemplary stalk-preferred promoters include the maize MS8-15 gene promoter (see, for example, U.S. Pat. No. 5,986,174 and International Publication No. WO 98/00533), and those found in Graham et al., (1997) Plant Mol Biol 33(4): 729-735.

Leaf-specific promoters may be used, such as those described in any of Yamamoto et al. Plant J., 1997, 12(2):255-265; Kwon et al., Plant Physiol., 1994, 105:357-67; Yamamoto et al., Plant Cell Physiol., 1994, 35(5):773-778; Gotor et al., Plant J., 1993, 3:509-18; Orozco et al., Plant Mol. Biol., 1993, 23(6):1129-1138; and Matsuoka et al., Proc. Natl. Acad. Sci. USA, 1993, 90(20):9586-9590.

Root-specific promoters may be used. Exemplary root-specific promoters include the soybean root-specific glutamine synthetase gene, a root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). Further root-specific promoters include the VfENOD-GRP3 gene promoter the rolB promoter. See also U.S. Pat. Nos. 5,837,876; 5,750,386; 5,633,363; 5,459,252; 5,401,836; 5,110,732; and 5,023,179, all of which are incorporated by reference.

“Seed-preferred” promoters include both “seed-specific” promoters (those promoters active during seed development such as promoters of seed storage proteins) as well as “seed-germinating” promoters (those promoters active during seed germination). See Thompson et al., (1989) BioEssays 10:108, herein incorporated by reference. Such seed-preferred promoters include, and are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is a preferred endosperm-specific promoter. Glob-1 is a preferred embryo-specific promoter. For dicots, seed-specific promoters include, and are not limited to, bean β-phaseolin, napin, β-conglycinin, soybean lectin, cruciferin, and the like. For monocots, seed-specific promoters include, and are not limited to, maize 15 kDa zein, 22 kDa zein, 27 kDa zein, g-zein, waxy, shrunken 1, shrunken 2, globulin 1, etc. See also WO 00/12733, where seed-preferred promoters from end1 and end2 genes are disclosed; herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al., Proc. Natl. Acad. Sci. USA, 1989, 86:6126-6130); potyvirus leaders, for example, TEV leader (Tobacco Etch Virus) (Gallie et al., Gene, 1995, 165(2):233-238), MDMV leader (Maize Dwarf Mosaic Virus), and human immunoglobulin heavy-chain binding protein (BiP) (Macejak et al., Nature, 1991, 353:90-94); untranslated leader from the coat protein mRNA of alfalfa mosaic virus (AMV RNA 4) (Jobling et al., Nature, 1987, 325:622-625); tobacco mosaic virus leader (TMV) (Gallie et al., Molecular Biology of RNA, ed. Cech (Liss, New York), 1989, pp. 237-256); and maize chlorotic mottle virus leader (MCMV) (Lommel et al., Virology, 1991, 81:382-385). See also, Della-Cioppa et al., Plant Physiol., 1987, 84:965-968. Other methods known to enhance translation can also be utilized, for example, introns, and the like.

In the expression cassette, the various DNA fragments may be configured to be in the proper orientation and, as appropriate, in the proper reading frame. Adapters or linkers may be employed to join the DNA fragments. Other modifications may be made to the expression cassette to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al., Biotechnol Bioeng, 2004, 85:610-9 and Fetter et al., Plant Cell, 2004, 16:215-28), cyan florescent protein (CYP) (Bolte et al., J. Cell Science, 2004, 117:943-54 and Kato et al., Plant Physiol, 2002, 129:913-42), and yellow florescent protein (PHIYFP™ fluorescent protein from Evrogen, see, Bolte et al., J. Cell Science, 2004, 117:943-54). For additional selectable markers, see generally, Yarranton, Curr. Opin. Biotech., 1992, 3:506-511; Christopherson et al., Proc. Natl. Acad. Sci. USA, 1992, 89:6314-6318; Yao et al., Cell, 1992, 71:63-72; Reznikoff, Mol. Microbiol., 1992, 6:2419-2422; Barkley et al., The Operon, 1980, pp. 177-220; Hu et al., Cell, 1987, 48:555-566; Brown et al., Cell, 1987, 49:603-612; Figge et al. (1988) Cell 52:713-722; Deuschle et al., Proc. Natl. Acad. Aci. USA, 1989, 86:5400-5404; Fuerst et al., Proc. Natl. Acad. Sci. USA, 1989, 86:2549-2553; Deuschle et al., Science, 1990, 248:480-483; Gossen (1993) Ph.D. Thesis, University of Heidelberg; Reines et al. Proc. Natl. Acad. Sci. USA, 1993, 90:1917-1921; Labow et al., Mol. Cell. Biol., 1990, 10:3343-3356; Zambretti et al., Proc. Natl. Acad. Sci. USA, 1992, 89:3952-3956; Baim et al., Proc. Natl. Acad. Sci. USA, 1991, 88:5072-5076; Wyborski et al., Nucleic Acids Res., 1991, 19:4647-4653; Hillenand-Wissman, Topics Mol. Struc. Biol., 1989, 10:143-162; Degenkolb et al., Antimicrob. Agents Chemother., 1991, 35:1591-1595; Kleinschnidt et al., Biochemistry, 1988, 27:1094-1104; Bonin (1993) Ph.D. Thesis, University of Heidelberg; Gossen et al., Proc. Natl. Acad. Sci. USA, 1992, 89:5547-5551; Oliva et al., Antimicrob. Agents Chemother., 1992, 36:913-919; Hlavka et al., Handbook of Experimental Pharmacology, 1985, Vol. 78 (Springer-Verlag, Berlin); Gill et al., Nature, 1988, 334:721-724. Such disclosures are herein incorporated by reference. The above list of selectable marker genes is not meant to be limiting. Any selectable marker gene can be used in the embodiments.

The gene of the embodiments can be expressed as a transgene in order to make plants resistant to pathogens, e.g., those that cause stalk rot. In some embodiments, the stalk rot is anthracnose stalk rot. In some embodiments, the stalk rot is Fusarium stalk rot. Using various different promoters described herein can allow for expression of the gene in a modulated form in different circumstances. For example, one might desire higher levels of expression in stalks to enhance resistance to stalk rot. In environments where leaf blight is a problem, lines with higher expression levels in leaves could be used. However, one can also insert the entire gene, both native promoter and coding sequence, as a transgene. Finally, using the gene of the embodiments as a transgene will allow quick combination with other traits, such as insect or herbicide resistance.

In certain embodiments, the nucleic acid sequences of the embodiments can be stacked with any combination of polynucleotide sequences of interest in order to create plants with a desired phenotype. This stacking may be accomplished by a combination of genes within the DNA construct, or by crossing with another line that comprises the combination. For example, the polynucleotides of the embodiments may be stacked with any other polynucleotides of the embodiments, or with other genes. The combinations generated can also include multiple copies of any one of the polynucleotides of interest. The polynucleotides of the embodiments can also be stacked with any other gene or combination of genes to produce plants with a variety of desired trait combinations including and not limited to traits desirable for animal feed such as high oil genes (e.g., U.S. Pat. No. 6,232,529); balanced amino acids (e.g. hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122); and high methionine proteins (Pedersen et al., J. Biol. Chem., 1986, 261:6279; Kirihara et al., Gene, 1988, 71:359; and Musumura et al., Plant Mol. Biol., 1989, 12: 123)); increased digestibility (e.g., modified storage proteins (U.S. Pat. No. 6,858,778); and thioredoxins (U.S. Pat. No. 7,009,087)), the disclosures of which are herein incorporated by reference. The polynucleotides of the embodiments can also be stacked with traits desirable for insect, disease or herbicide resistance (e.g., Bacillus thuringiensis toxic proteins (U.S. Pat. Nos. 5,366,892; 5,747,450; 5,737,514; 5,723,756; 5,593,881; Geiser et al., Gene, 1986, 48:109); lectins (Van Damme et al., Plant Mol. Biol., 1994, 24:825); fumonisin detoxification genes (U.S. Pat. No. 5,792,931); avirulence and disease resistance genes (Jones et al., Science, 1994, 266:789; Martin et al., Science, 1993, 262:1432; Mindrinos et al., Cell, 1994, 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS genes, GAT genes such as those disclosed in U.S. Pat. No. 7,462,481, also WO02/36782 and WO03/092360)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers orbioplastics (e.g., U.S. Pat. No. 5,602,321; beta-ketothiolase, polyhydroxybutyrate synthase, and acetoacetyl-CoA reductase (Schubert et al., J. Bacteriol. 1988, 170:5837-5847) facilitate expression of polyhydroxyalkanoates (PHAs)), the disclosures of which are herein incorporated by reference. One could also combine the polynucleotides of the embodiments with polynucleotides providing agronomic traits such as male sterility (e.g., see U.S. Pat. No. 5,583,210), stalk strength, flowering time, or transformation technology traits such as cell cycle regulation or gene targeting (e.g. WO 99/61619; WO 00/17364; WO 99/25821), the disclosures of which are herein incorporated by reference.

These stacked combinations can be created by any method including and not limited to cross breeding plants by any conventional or TOPCROSS® breeding methodology, or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant.

Introducing Polypeptides or Polynucleotides into Plants

The methods of the embodiments may involve, and are not limited to, introducing a polypeptide or polynucleotide into a plant or plant cell. “Introducing” is intended to mean presenting to the plant the polynucleotide. In some embodiments, the polynucleotide will be presented in such a manner that the sequence gains access to the interior of a cell of the plant, including its potential insertion into the genome of a plant. The methods of the embodiments do not depend on a particular method for introducing a sequence into a plant, only that the polynucleotide gains access to the interior of at least one cell of the plant. Methods for introducing polynucleotides into plants are known in the art including, and not limited to, stable transformation methods, transient transformation methods, and virus-mediated methods.

In various embodiments, one or more of the following polynucleotides may be introduced into the plant:
i. a nucleotide sequence of SEQ ID NO: 209, 212, 215, 218, 221, 224, 227, 230, 233, 236, 239, 242, 245, 248, 251, 254, 257, 260, 263, 266 or 269, preferably of SEQ ID NO: 266 or 269,
ii. a nucleotide sequence having a coding sequence of SEQ ID NO: 210, 213, 216, 219, 222, 225, 228, 231, 234, 237, 240, 243, 246, 249, 252, 255, 258, 261, 264, 267 or 270, preferably of SEQ ID NO: 267 or 270,
iii. a nucleotide sequence which is complementary to a sequence from i. or ii.,
iv. a nucleotide sequence which has at least 80% or 85%, preferably at least 90%, 91%, 92%, 93%, 94% or 95%, more preferably at least 96%, 97%, 98% or 99% identity with a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 211, 214, 217, 220, 223, 226, 229, 232, 235, 238, 241, 244, 247, 250, 253, 256, 259, 262, 265, 268 or 271, preferably which encodes for a protein comprising the sequence of SEQ ID NO: 268 or 271, or
viii. a nucleotide sequence which encodes for a protein which has at least 80% or 85%, preferably at least 90%, 91%, 92%, 93%, 94% or 95%, more preferably at least 960%, 97%, 98% or 99% identity with a sequence of SEQ ID NO: 211, 214, 217, 220, 223, 226, 229, 232, 235, 238, 241, 244, 247, 250, 253, 256, 259, 262, 265, 268 or 271, preferably with a sequence of SEQ ID NO: 268 or 271.

In various embodiments, a polypeptide comprising a sequence at least 80%, 81%, 82%, 83%, 84%, 85%, 86%, 87%, 88%, 89%, 90%, 91%, 92%, 93%, 94%, 95%, 96%, 97%, 98%, 99% or 100% identity to SEQ ID NO: 211, 214, 217, 220, 223, 226, 229, 232, 235, 238, 241, 244, 247, 250, 253, 256, 259, 262, 265, 268 or 271, preferably to SEQ ID NO: 268 or 271, is introduced into the plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al., Biotechniques, 1986, 4:320-334), electroporation (Riggs et al., Proc. Natl. Acad. Sci. USA, 1986, 83:5602-5606, Agrobacterium-mediated transformation (U.S. Pat. Nos. 5,563,055- and 5,981,840), direct gene transfer (Paszkowski et al., EMBO J., 1984, 3:2717-2722), and ballistic particle acceleration (see, for example, Sanford et al., U.S. Pat. Nos. 4,945,050; 5,879,918; 5,886,244; and U.S. Pat. No. 5,932,782; McCabe et al., Biotechnology, 1988, 6:923-926); and Lec transformation (WO 00/28058). Also see, Weissinger et al., Ann. Rev. Genet., 1988, 22:421-477; Sanford et al., Particulate Science and Technology, 1987, 5:27-37 (onion); Christou et al., Plant Physiol., 1988, 87:671-674 (soybean); McCabe et al., Bio/Technology, 1988, 6:923-926 (soybean); Finer and McMullen, In Vitro Cell Dev. Biol., 1991, 27P:175-182 (soybean); Singh et al., Theor. Appl. Genet., 1998, 96:319-324 (soybean); Datta et al., Biotechnology, 1990, 8:736-740 (rice); Klein et al., Proc. Natl. Acad. Sci. USA, 1988, 85:4305-4309 (maize); Klein et al., Biotechnology, 1988, 6:559-563 (maize); U.S. Pat. Nos. 5,240,855; 5,322,783 and 5,324,646; Klein et al., Plant Physiol., 1988, 91:440-444 (maize); Fromm et al., Biotechnology, 1990, 8:833-839 (maize); Hooykaas-Van Slogteren et al., Nature, 1984, (London) 311:763-764; U.S. Pat. No. 5,736,369; Bytebier et al., Proc. Natl. Acad. Sci. USA, 1987, 84:5345-5349 (Liliaceae); De Wet et al., The Experimental Manipulation of Ovule Tissues, ed. Chapman et al. (Longman, New York), 1985, pp. 197-209 (pollen); Kaeppler et al., Plant Cell Reports, 1990, 9:415-418 and Kaeppler et al., Theor. Appl. Genet., 1992, 84:560-566 (whisker-mediated transformation); D'Halluin et al., Plant Cell, 1992, 4:1495-1505 (electroporation); Li et al., Plant Cell Reports, 1993, 12:250-255 and Christou and Ford, Annals of Botany, 1995, 75:407-413 (rice); Osjoda et al., Nature Biotechnology, 1996, 14:745-750 (maize via Agrobacterium tumefaciens); all of which are herein incorporated by reference.

Methods for the targeted insertion of a polynucleotide at a specific location in the plant genome are known in the art. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide of the embodiments can be contained in transfer cassette flanked by two non-identical recombination sites. The transfer cassette is introduced into a plant or a plant cell have stably incorporated into its genome a target site which is flanked by two non-identical recombination sites that correspond to the sites of the transfer cassette. An appropriate recombinase is provided and the transfer cassette is integrated at the target site. The polynucleotide of interest may then be integrated from the transfer cassette to a specific chromosomal position in the plant genome.

The cells that have been transformed may be grown into plants in accordance with conventional ways. See, for example, McCormick et al., Plant Cell Reports, 1986, 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the embodiments provides transformed seed (also referred to as “transgenic seed”) having a nucleotide construct of the embodiments, for example, an expression cassette of the embodiments, stably incorporated into their genome.

These embodiments may be used to confer or enhance fungal plant pathogen resistance or protect from fungal pathogen attack in plants, especially corn (Zea mays). Different parts of the plant may be protected from attack by pathogens, with the parts including and not limited to stalks, ears, leaves, roots and tassels. Other plant species may also be of interest in practicing the embodiments of the disclosure, including, and not limited to, other monocot crop plants.

Where appropriate, the polynucleotides may be optimized for increased expression in the transformed organism. For example, the polynucleotides can be synthesized using plant-preferred codons for improved expression. Methods are available in the art for synthesizing plant-preferred genes. See, for example, U.S. Pat. Nos. 5,380,831, and 5,436,391, and Murray et al., Nucleic Acids Res., 1989, 17:477-498, herein incorporated by reference.

The embodiments described herein may be effective against a variety of plant pathogens, particularly fungal pathogens, such as, for example Colletotrichum graminicola Ces. Wils. (Cg), Fusarium verticilliodes, and related species. The embodiments of the present disclosure may also be effective against maize stalk rot, including anthracnose stalk rot, wherein the causative agent is Colletotrichum graminicola Ces. Wils., Fusarium verticilliodes, and related species. In some embodiments, the stalk rot is Fusarium stalk rot.

Hence, the methods of the embodiments can be utilized to protect plants from disease, particularly those diseases that are caused by plant fungal pathogens. Fungal resistance can provide for enhanced resistance or tolerance to a fungal pathogen when compared to that of a wild type plant. Effects may vary from a slight increase in tolerance to the effects of the fungal pathogen (e.g., partial inhibition) to total resistance such that the plant is unaffected by the presence of the fungal pathogen. An increased level of resistance against a particular fungal pathogen or against a wider spectrum of fungal pathogens constitutes “enhanced” or improved fungal resistance. The embodiments of the disclosure also will enhance or improve fungal plant pathogen resistance, such that the resistance of the plant to a fungal pathogen or pathogens will increase. The term “enhance” refers to improve, increase, amplify, multiply, elevate, raise, and the like. Herein, plants of the disclosure are described as being resistant to infection by fungi such as Colletotrichum graminicola Ces. Wils., Fusarium verticilliodes, and related species or having ‘enhanced resistance’ to infection by Colletotrichum graminicola Ces. Wils., Fusarium verticilliodes, and related species as a result of the loci at chromosomes 4 and 6 conferring the resistance to plant, as described herein. Resistance may be provided by only a locus at chromosome 4. Resistance may be provided by only a locus at chromosome 6. Resistance may be provided by a locus at chromosome 4 and a locus at chromosome 6 together.

### Methods of Identifying and Selecting Maize Plants Displaying Resistance to Stalk Rot

In one aspect is provided a process of identifying a maize plant that displays enhanced resistance to anthracnose stalk rot, the process comprising detecting in the maize plant
a. the presence of at least two markers at a resistance locus on chromosome 6 comprising a “G” at C16759-001-K1 and one of the following single nucleotide polymorphisms:
   a “C” at C12305-001-K1,
   a “C” at C12307-001-K1,
   a “G” at C16760-001-K1,
   an “A” at C12314-001-K1; and/or
b. the presence of at least one marker at a resistance locus on chromosome 6 comprising at least one of the variant nucleotide polymorphisms recited in Table 13, and/or
c. the presence of at least one marker at a resistance locus on chromosome 4, wherein the resistance locus comprises a Rcg1 resistance allele having a haplotype comprising one or more single nucleotide polymorphism selected from the group consisting of:
   a “C” at position 413 referenced to SEQ ID NO: 50,
   a “C” at position 958 referenced to SEQ ID NO: 50,
   a “C” at position 971 referenced to SEQ ID NO: 50,
   a “T” at position 1099 referenced to SEQ ID NO: 50,
   an “A” at position 1154 referenced to SEQ ID NO: 50,
   a “T” at position 1250 referenced to SEQ ID NO: 50,
   a “G” at position 1607 referenced to SEQ ID NO: 50,
   a “G” at position 2001 referenced to SEQ ID NO: 50,
   an “A” at position 2598 referenced to SEQ ID NO: 50, and
   an “A” at position 3342 referenced to SEQ ID NO: 50,
      where the at least two markers of (a) and/or the at least one marker of (b) are closely linked to and associated with the resistance locus on chromosome 6 and the at least one marker of (c) is closely linked to and associated with the resistance locus on chromosome 4.

In various embodiments, the resistance locus on chromosome 6 is located on a chromosomal interval between makers PZE-106066805 and PZE-106075546. In various embodiments, the resistance locus on chromosome 4 is located on a chromosomal interval between makers PZE-104102206 and PZE-104132759. In various embodiments, the resistance locus on chromosome 6 is located on a chromosomal interval between 139646631 and 139889078 numbered according to the B73AGPv05 genome sequence. In various embodiments, the resistance locus of chromosome 6 comprises SEQ ID NO: 272, or a fragment thereof. In various embodiments, the resistance locus on chromosome 6 comprising one or more nucleotide sequences is selected from the group consisting of
i. a nucleotide sequence of SEQ ID NO: 209, 212, 215, 218, 221, 224, 227, 230, 233, 236, 239, 242, 245, 248, 251, 254, 257, 260, 263, 266 or 269, preferably of SEQ ID NO: 266 or 269,
ii. a nucleotide sequence having a coding sequence of 210, 213, 216, 219, 222, 225, 228, 231, 234, 237, 240, 243, 246, 249, 252, 255, 258, 261, 264, 267 or 270, preferably of SEQ ID NO: 267 or 270,
iii. a nucleotide sequence which is complementary to a sequence from i. or ii.,
iv. a nucleotide sequence which has at least 80% or 85%, preferably at least 90%, 91%, 92%, 93%, 94% or 95%, more preferably at least 96%, 97%, 98% or 99% identity with a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 211, 214, 217, 220, 223, 226, 229, 232, 235, 238, 241, 244, 247, 250, 253, 256, 259, 262, 265, 268 or 271, preferably the sequence of SEQ ID NO: 268 or 271, or
viii. a nucleotide sequence which encodes for a protein which has at least 80% or 85%, preferably at least 90%, 91%, 92%, 93%, 94% or 95%, more preferably at least 960%, 97%, 98% or 99% identity with the sequence of SEQ ID NOs: 211, 214, 217, 220, 223, 226, 229, 232, 235, 238, 241, 244, 247, 250, 253, 256, 259, 262, 265, 268 or 271, preferably with the sequence of SEQ ID NO: 268 or 271.

In various embodiments, the resistance locus on chromosome 6 comprising one or more nucleotide sequences is selected from the group consisting of
i. a nucleotide sequence of SEQ ID NO: 1, 20, 23, 44 or 47,
ii. a nucleotide sequence which is complementary to a sequence from i.,
iii. a nucleotide sequence which has at least 80% or 85%, preferably at least 90%, 91%, 92%, 93%, 94% or 95%, more preferably at least 96%, 97%, 98% or 99% identity with a sequence from i. or ii.,
iv. a nucleotide sequence which differs from a nucleic acid sequence according to i., ii. or iii. depending on the degeneracy of the genetic code,
v. a nucleotide sequence which hybridizes with a nucleic acid sequence according to i., ii. or iii. under stringent conditions,
vi. a nucleotide sequence which encodes for a protein comprising the sequence of SEQ ID NO: 11-19, 22, 34-43, 46 or 49, or
vii. a nucleotide sequence which encodes for a protein which has at least 80% or 85%, preferably at least 90%, 91%, 92%, 93%, 94% or 95%, more preferably at least 960%, 97%, 98% or 99% identity with the sequence of SEQ ID NOs: 11-19, 22, 34-43, 46 or 49.

In various embodiments, the resistance locus on chromosome 4 does not produce the amplicon according to SEQ ID NO: 119 (amplicon 7) upon polymerase chain reaction amplification by means of primers of SEQ ID NOs: 120 and 121, primers of SEQ ID NOs: 122 and 123, or primers of SEQ ID NOs: 124 and 125.

In various embodiments, the resistance locus on chromosome 4 does not produce an amplicon selected from the group consisting of SEQ ID NOs: 94, 101, 106, 109, and 114 (from each of amplicons 2-6, respectively) upon polymerase chain reaction amplification by means of primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118.

In various embodiments, the resistance locus on chromosome 6 is derived from NC262A. In various embodiments, the resistance locus on chromosome 4 is derived from NC262A or NC342. In various embodiments, the process comprising detecting in the maize plant both (A) the presence or absence of at least one allele at the resistance locus on chromosome 6 as defined above under b. and (B) the presence or absence of at least one marker at the resistance locus on chromosome 4 as defined above under c.

In various embodiments, the at least one marker at the resistance locus on chromosome 6 detects a “G” at C16759-001-K1.

In various embodiments, the at least one marker at the resistance locus on chromosome 6 detects at least one of the variant nucleotide polymorphisms listed in Table 13. In various embodiments, the at least one marker at the resistance locus on chromosome 6 detects at least one of the variant nucleotide polymorphisms of the “A” at position 132836954 on chromosome 6, the “T” at position 132836944 on chromosome 6, an “A” at position 132836869 on chromosome 6, the “T” at position 132836849 on chromosome 6, the “T” at position 132836845 on chromosome 6, the “G” at position 132836840 on chromosome 6, the “T” at position 132836830 on chromosome 6, an “A” at position 132836810 on chromosome 6, the “CA” at position 132836805 on chromosome 6, the deletion of “ATC” at position 132836802 on chromosome 6, the deletion of “ATT” at position 132836799 on chromosome 6, an “A” at position 132836791 on chromosome 6, the “CTG” at position 132836787 on chromosome 6, the “GA” at position 132836781 on chromosome 6, the “T” at position 132836776 on chromosome 6, the “AG” at position 132836767 on chromosome 6, an “A” at position 132836762 on chromosome 6, the insertion of “CGCCAA” and an “A” at position 132836679 on chromosome 6, an “A” at position 132836678 on chromosome 6, the “G” at position 132836670 on chromosome 6, the “T” at position 132836667 on chromosome 6, an “A” at position 132836665 on chromosome 6, the “C” at position 132836662 on chromosome 6, the “T” at position 132836660 on chromosome 6, the “G” at position 132836651 on chromosome 6, the deletion of “AAT” at position 132836643 on chromosome 6, the deletion of “GCCATG” at position 132836636 on chromosome 6, the “AG” at position 132836622 on chromosome 6, the “AC” at position 132836620 on chromosome 6, the “G” at position 132836612 on chromosome 6, the “G” at position 132836603 on chromosome 6, the “C” at position 132836591 on chromosome 6, an “A” at position 132836586 on chromosome 6, the “G” at position 132836580 on chromosome 6, an “A” at position 132836069 on chromosome 6, an “A” at position 132836063 on chromosome 6, the “G” at position 132836061 on chromosome 6, the “G” at position 132836056 on chromosome 6, the “C” at position 132836050 on chromosome 6, the “CGT” at position 132836047 on chromosome 6, the “G” at position 132836034 on chromosome 6, the “G” at position 132836031 on chromosome 6, the “T” at position 132836019 on chromosome 6, the “G” at position 132836008 on chromosome 6, the “G” at position 132835978 on chromosome 6, the “G” at position 132835910 on chromosome 6, the “G” at position 132835851 on chromosome 6, the “C” at position 132835819 on chromosome 6, the “G” at position 132835803 on chromosome 6, the “T” at position 132835793 on chromosome 6, the “C” at position 132835788 on chromosome 6, an “A” at position 132835781 on chromosome 6, an “A” at position 132835779 on chromosome 6, the “T” at position 132835776 on chromosome 6, the “G” at position 132835746 on chromosome 6, an “A” at position 132835729 on chromosome 6, the “C” at position 132835728 on chromosome 6, the insertion of “GACATC” at position 132835722 on chromosome 6, the “T” at position 132835713 on chromosome 6, the deletion of “GAA” at position 132835708 on chromosome 6, the deletion of “ATA” at position 132835705 on chromosome 6, the deletion of “GAT” at position 132835702 on chromosome 6, the “G” at position 132835693 on chromosome 6, the “T” at position 132835681 on chromosome 6, the “C” at position 132835671 on chromosome 6, the “T” at position 132835666 on chromosome 6, an “A” at position 132835271 on chromosome 6, the “GG” at position 132835267 on chromosome 6, the “T” at position 132835147 on chromosome 6, the “C” at position 132835031 on chromosome 6, an “A” at position 132834960 on chromosome 6, an “A” at position 132834951 on chromosome 6, the “AC” at position 132834946 on chromosome 6, an “A” at position 132834942 on chromosome 6, an “A” at position 132834929 on chromosome 6, the “T” at position 132834927 on chromosome 6, an “A” at position 132834924 on chromosome 6, the “C” at position 132834919 on chromosome 6, the “T” at position 132834908 on chromosome 6, an “A” at position 132834899 on chromosome 6, the “C” at position 132834754 on chromosome 6, the “C” at position 132834753 on chromosome 6, the “C” at position 132834748 on chromosome 6, an “A” at position 132834721 on chromosome 6, an “A” at position 132834632 on chromosome 6, the “C” at position 132834622 on chromosome 6, the “G” at position 132834615 on chromosome 6, the “C” at position 132834602 on chromosome 6, the “G” at position 132834589 on chromosome 6, the “T” at position 132834581 on chromosome 6, an “A” at position 132834577 on chromosome 6, the “G” at position 132834569 on chromosome 6, the “C” at position 132834539 on chromosome 6, the “GAG” at position 132834532 on chromosome 6, the “T” at position 132834494 on chromosome 6, the “T” at position 132834431 on chromosome 6, the “G” at position 132834389 on chromosome 6, the “T” at position 132834208 on chromosome 6, the “T” at position 132831963 on chromosome 6, the “G” at position 132831924 on chromosome 6, an “A” at position 132831915 on chromosome 6, the “G” at position 132831839 on chromosome 6, the “T” at position 132829096 on chromosome 6, an “A” at position 132828997 on chromosome 6, the “G” at position 132828958 on chromosome 6, the “G” at position 132828950 on chromosome 6, the “C” at position 132828867 on chromosome 6, an “A” at position 132827606 on chromosome 6, the insertion of “AGTTCATAATAAAGTGATAGAGTT” (SEQ ID NO: 414) at position 132827596 on chromosome 6, the “T” at position 132827573 on chromosome 6, the “G” at position 132827566 on chromosome 6, an “A” at position 132827549 on chromosome 6, the “TAT” at position 132827545 on chromosome 6, the “C” at position 132827302 on chromosome 6, the “T” at position 132826983 on chromosome 6, the “T” at position 132826940 on chromosome 6, the “T” at position 1333040380 on chromosome 6, the “C” at position 1333040382 on chromosome 6, the “T” at position 133040760 on chromosome 6, compared to the position(s) on chromosome 6 of the B73AGPv04 reference genome sequence. In various embodiments, the at least one marker at the resistance locus on chromosome 6 are selected from the group consisting of SEQ ID NO: 273 to 407.

In various embodiments, the at least one marker at the resistance locus on chromosome 4 detects the nucleotide polymorphisms of the “C” at position 413 in SEQ ID NO: 50, the “C” at position 958 in SEQ ID NO: 50, the “C” at position 971 in SEQ ID NO: 50, the “T” at position 1099 in SEQ ID NO: 50, the “A” at position 1154 in SEQ ID NO: 50, the “T” at position 1250 in SEQ ID NO: 50, the “G” at position 1607 in SEQ ID NO: 50, the “G” at position 2001 in SEQ ID NO: 50, the “A” at position 2598 in SEQ ID NO: 50, or the “A” at position 3342 in SEQ ID NO: 50.

The presence or absence of at least one nucleotide polymorphism may be detected by polymerase chain reaction amplification of a nucleic acid present in the maize plant with a primer configured to specifically amplify a nucleic acid sequence comprising one or more of the single nucleotide polymorphisms described herein. The single nucleotide polymorphism may be a “C” at position 413 in SEQ ID NO: 50, and the primer may comprise the sequence GTACCATGTGACCA (SEQ ID NO: 406). An example of such primer is a primer comprising the sequence of SEQ ID NO: 58. The single nucleotide polymorphism may be a “T” at position 1099 in SEQ ID NO: 50, and the primer may comprise the sequence GTAGTGTTTTGAC (SEQ ID NO: 407). An example of such primer is a primer comprising the sequence of SEQ ID NO: 61. The single nucleotide polymorphism may be a “T” at position 1250 in SEQ ID NO: 50, and the primer may comprise the sequence TGATCTCAAAGAT (SEQ ID NO: 408). An example of such primer is a primer comprising the sequence of SEQ ID NO: 64. The single nucleotide polymorphism may be a “G” at position 1607 in SEQ ID NO: 50, and the primer may comprise the sequence GTTATGTGCACAA (SEQ ID NO: 409). An example of such primer is a primer comprising the sequence of SEQ ID NO: 67. The single nucleotide polymorphism may be a “G” at position 2001 in SEQ ID NO: 50, and the primer may comprise the sequence AGATGAAGGCTGT (SEQ ID NO: 410). An example of such primer is a primer comprising the sequence of SEQ ID NO: 70. The single nucleotide polymorphism may be an “A” at position 2598 in SEQ ID NO: 50, and the primer may comprise the sequence AAGTGACATGCAG (SEQ ID NO: 411). An example of such primer is a primer comprising the sequence of SEQ ID NO: 73. The single nucleotide polymorphism may be an “A” at position 3342 in SEQ ID NO: 50, and the primer may comprise the sequence CATCTGATGAAAGC (SEQ ID NO: 412). An example of such primer is a primer comprising the sequence of SEQ ID NO: 76.

In some embodiments, the presence or absence of the allele comprising a “G” at C16759-001-K1 is detected by polymerase chain reaction amplification of a nucleic acid present in the maize plant with a primer configured to specifically amplify a nucleic acid sequence of the allele. In some embodiments, the primer comprises the sequence AATTATGCTGATGA (SEQ ID NO: 413). An example of such primer is a primer comprising the sequence of SEQ ID NO: 85.

In another aspect is provided a process of selecting a maize plant with anthracnose stalk rot resistance, the process comprising identifying the maize plant according to any of the above-described processes, and selecting the maize plant as having anthracnose stalk rot resistance if the presence or absence of the at least one marker at the resistance locus on chromosome 6 and/or the at least one marker at the resistance locus on chromosome 4 is detected. A maize plant may be selected that comprises at least one additional marker allele that is closely linked to and associated with the nucleotide polymorphism(s). The additional marker allele may be linked to the nucleotide polymorphism by no more than 2 cM on a single meiosis based genetic map. The process may further comprise selecting the maize plant that comprises at least one additional marker allele that is linked to and associated with the allele comprising a “G” at C16759-001-K1. The additional marker allele may be linked to an allele comprising a “G” at C16759-001-K1 by no more than 2 cM on a single meiosis based genetic map. The additional marker allele may be linked to an allele comprising at least one of the variant nucleotide polymorphisms recited in Table 13.

In various embodiments, the process further comprises backcrossing the identified maize plant with another maize plant, preferably comprising backcrossing the resistance locus on chromosome 6 into a genotype which is not NC262A and/or the resistance locus on chromosome 4 into a genotype which is not NC262A or NC342.

In one aspect is provided a method of selecting a maize plant that displays resistance to anthracnose stalk rot. A first maize plant is obtained that comprises within its genome a haplotype comprising one or more polymorphism of
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50,
x. an “A” at position 3342 referenced to SEQ ID NO: 50, and/or
xi. a “G” at C16759-001-K1; and/or
xii. an “A” at position 132836954 on chromosome 6 referenced to B73AGPv04, preferably detectable by marker sequence of SEQ ID NO: 273,
xiii. a “T” at position 132836944 on chromosome 6 referenced to B73AGPv04, preferably detectable by marker sequence of SEQ ID NO: 274,
xiv. an “A” at position 132836869 on chromosome 6 referenced to B73AGPv04, preferably detectable by marker sequence of SEQ ID NO: 275,
xv. a “T” at position 132836849 on chromosome 6 referenced to B73AGPv04, preferably detectable by marker sequence of SEQ ID NO: 276,
xvi. a “T” at position 132836845 on chromosome 6 referenced to B73AGPv04, preferably detectable by marker sequence of SEQ ID NO: 277,
xvii. a “G” at position 132836840 on chromosome 6 referenced to B73AGPv04, preferably detectable by marker sequence of SEQ ID NO: 278,
xviii. a “T” at position 132836830 on chromosome 6 referenced to B73AGPv04, preferably detectable by marker sequence of SEQ ID NO: 279,
xix. an “A” at position 132836810 on chromosome 6 referenced to B73AGPv04, preferably detectable by marker sequence of SEQ ID NO: 280,
xx. a “CA” at position 132836805 on chromosome 6 referenced to B73AGPv04, preferably detectable by marker sequence of SEQ ID NO: 281 and/or 282,
xxi. a deletion of “ATC” at position 132836802 on chromosome 6 referenced to B73AGPv04, preferably detectable by marker sequence of SEQ ID NO: 283 and/or 284,
xxii. a deletion of “ATT” at position 132836799 on chromosome 6 referenced to B73AGPv04, preferably detectable by marker sequence of SEQ ID NO: 283 and/or 284,
xxiii. an “A” at position 132836791 on chromosome 6 referenced to B73AGPv04, preferably detectable by marker sequence of SEQ ID NO: 285,
xxiv. a “CTG” at position 132836787 on chromosome 6 referenced to B73AGPv04, preferably detectable by marker sequence of SEQ ID NO: 286 and/or 287,
xxv. a “GA” at position 132836781 on chromosome 6 referenced to B73AGPv04, preferably detectable by marker sequence of SEQ ID NO: 288 and/or 289,
xxvi. a “T” at position 132836776 on chromosome 6 referenced to B73AGPv04, preferably detectable by marker sequence of SEQ ID NO: 290,
xxvii. a “AG” at position 132836767 on chromosome 6 referenced to B73AGPv04, preferably detectable by marker sequence of SEQ ID NO: 291 and/or 292,
xxviii. an “A” at position 132836762 on chromosome 6 referenced to B73AGPv04, preferably detectable by marker sequence of SEQ ID NO: 293,
xxix. an insertion of “CGCCAA” and an “A” at position 132836679 on chromosome 6 referenced to B73AGPv04, preferably detectable by marker sequence of SEQ ID NO: 294 and/or 295,
xxx. an “A” at position 132836678 on chromosome 6 referenced to B73AGPv04, preferably detectable by marker sequence of SEQ ID NO: 296,
xxxi. a “G” at position 132836670 on chromosome 6 referenced to B73AGPv04, preferably detectable by marker sequence of SEQ ID NO: 297,
xxxii. a “T” at position 132836667 on chromosome 6 referenced to B73AGPv04, preferably detectable by marker sequence of SEQ ID NO: 298,
xxxiii. an “A” at position 132836665 on chromosome 6 referenced to B73AGPv04, preferably detectable by marker sequence of SEQ ID NO: 299,
xxxiv. a “C” at position 132836662 on chromosome 6 referenced to B73AGPv04, preferably detectable by marker sequence of SEQ ID NO: 300,
xxxv. a “T” at position 132836660 on chromosome 6 referenced to B73AGPv04, preferably detectable by marker sequence of SEQ ID NO: 301,
xxxvi. a “G” at position 132836651 on chromosome 6 referenced to B73AGPv04, preferably detectable by marker sequence of SEQ ID NO: 302,
xxxvii. a deletion of “AAT” at position 132836643 on chromosome 6 referenced to B73AGPv04, preferably detectable by marker sequence of SEQ ID NO: 303 and/or 304,
xxxviii. a deletion of “GCCATG” at position 132836636 on chromosome 6 referenced to B73AGPv04, preferably detectable by marker sequence of SEQ ID NO: 305 and/or 306,
xxxix. a “AG” at position 132836622 on chromosome 6 referenced to B73AGPv04, preferably detectable by marker sequence of SEQ ID NO: 307 and/or 308,
xl. a “AC” at position 132836620 on chromosome 6 referenced to B73AGPv04, preferably detectable by marker sequence of SEQ ID NO: 309 and/or 310,
xli. a “G” at position 132836612 on chromosome 6 referenced to B73AGPv04, preferably detectable by marker sequence of SEQ ID NO: 311,
xlii. a “G” at position 132836603 on chromosome 6 referenced to B73AGPv04, preferably detectable by marker sequence of SEQ ID NO: 312,
xliii. a “C” at position 132836591 on chromosome 6 referenced to B73AGPv04, preferably detectable by marker sequence of SEQ ID NO: 313,
xliv. an “A” at position 132836586 on chromosome 6 referenced to B73AGPv04, preferably detectable by marker sequence of SEQ ID NO: 314,
xlv. a “G” at position 132836580 on chromosome 6 referenced to B73AGPv04, preferably detectable by marker sequence of SEQ ID NO: 315,
xlvi. an “A” at position 132836069 on chromosome 6 referenced to B73AGPv04, preferably detectable by marker sequence of SEQ ID NO: 316,
xlvii. an “A” at position 132836063 on chromosome 6 referenced to B73AGPv04, preferably detectable by marker sequence of SEQ ID NO: 317,
xlviii. a “G” at position 132836061 on chromosome 6 referenced to B73AGPv04, preferably detectable by marker sequence of SEQ ID NO: 318,
xlix. a “G” at position 132836056 on chromosome 6 referenced to B73AGPv04, preferably detectable by marker sequence of SEQ ID NO: 319,
l. a “C” at position 132836050 on chromosome 6 referenced to B73AGPv04, preferably detectable by marker sequence of SEQ ID NO: 320,
li. a “CGT” at position 132836047 on chromosome 6 referenced to B73AGPv04, preferably detectable by marker sequence of SEQ ID NO: 321 and/or 322,
lii. a “G” at position 132836034 on chromosome 6 referenced to B73AGPv04, preferably detectable by marker sequence of SEQ ID NO: 323,
liii. a “G” at position 132836031 on chromosome 6 referenced to B73AGPv04, preferably detectable by marker sequence of SEQ ID NO: 324,
liv. a “T” at position 132836019 on chromosome 6 referenced to B73AGPv04, preferably detectable by marker sequence of SEQ ID NO: 325,
lv. a “G” at position 132836008 on chromosome 6 referenced to B73AGPv04, preferably detectable by marker sequence of SEQ ID NO: 326,
lvi. a “G” at position 132835978 on chromosome 6 referenced to B73AGPv04, preferably detectable by marker sequence of SEQ ID NO: 327,
lvii. a “G” at position 132835910 on chromosome 6 referenced to B73AGPv04, preferably detectable by marker sequence of SEQ ID NO: 328,
lviii. a “G” at position 132835851 on chromosome 6 referenced to B73AGPv04, preferably detectable by marker sequence of SEQ ID NO: 329,
lix. a “C” at position 132835819 on chromosome 6 referenced to B73AGPv04, preferably detectable by marker sequence of SEQ ID NO: 330,
lx. a “G” at position 132835803 on chromosome 6 referenced to B73AGPv04, preferably detectable by marker sequence of SEQ ID NO: 331,
lxi. a “T” at position 132835793 on chromosome 6 referenced to B73AGPv04, preferably detectable by marker sequence of SEQ ID NO: 332,
lxii. a “C” at position 132835788 on chromosome 6 referenced to B73AGPv04, preferably detectable by marker sequence of SEQ ID NO: 333,
lxiii. an “A” at position 132835781 on chromosome 6 referenced to B73AGPv04, preferably detectable by marker sequence of SEQ ID NO: 334,
lxiv. an “A” at position 132835779 on chromosome 6 referenced to B73AGPv04, preferably detectable by marker sequence of SEQ ID NO: 335,
lxv. a “T” at position 132835776 on chromosome 6 referenced to B73AGPv04, preferably detectable by marker sequence of SEQ ID NO: 336,
lxvi. a “G” at position 132835746 on chromosome 6 referenced to B73AGPv04, preferably detectable by marker sequence of SEQ ID NO: 337,
lxvii. an “A” at position 132835729 on chromosome 6 referenced to B73AGPv04, preferably detectable by marker sequence of SEQ ID NO: 338,
lxviii. a “C” at position 132835728 on chromosome 6 referenced to B73AGPv04, preferably detectable by marker sequence of SEQ ID NO: 339,
lxix. an insertion of “GACATC” at position 132835722 on chromosome 6 referenced to B73AGPv04, preferably detectable by marker sequence of SEQ ID NO: 340 and/or 341,
lxx. a “T” at position 132835713 on chromosome 6 referenced to B73AGPv04, preferably detectable by marker sequence of SEQ ID NO: 342,
lxxi. a deletion of “GAA” at position 132835708 on chromosome 6 referenced to B73AGPv04, preferably detectable by marker sequence of SEQ ID NO: 343 and/or 344,
lxxii. a deletion of “ATA” at position 132835705 on chromosome 6 referenced to B73AGPv04, preferably detectable by marker sequence of SEQ ID NO: 343 and/or 344,
lxxiii. a deletion of “GAT” at position 132835702 on chromosome 6 referenced to B73AGPv04, preferably detectable by marker sequence of SEQ ID NO: 343 and/or 344,
lxxiv. a “G” at position 132835693 on chromosome 6 referenced to B73AGPv04, preferably detectable by marker sequence of SEQ ID NO: 345,
lxxv. a “T” at position 132835681 on chromosome 6 referenced to B73AGPv04, preferably detectable by marker sequence of SEQ ID NO: 346,
lxxvi. a “C” at position 132835671 on chromosome 6 referenced to B73AGPv04, preferably detectable by marker sequence of SEQ ID NO: 347,
lxxvii. a “T” at position 132835666 on chromosome 6 referenced to B73AGPv04, preferably detectable by marker sequence of SEQ ID NO: 348,
lxxviii. an “A” at position 132835271 on chromosome 6 referenced to B73AGPv04, preferably detectable by marker sequence of SEQ ID NO: 349,
lxxix. a “GG” at position 132835267 on chromosome 6 referenced to B73AGPv04, preferably detectable by marker sequence of SEQ ID NO: 350 and/or 351,
lxxx. a “T” at position 132835147 on chromosome 6 referenced to B73AGPv04, preferably detectable by marker sequence of SEQ ID NO: 352,
lxxxi. a “C” at position 132835031 on chromosome 6 referenced to B73AGPv04, preferably detectable by marker sequence of SEQ ID NO: 353,
lxxxii. an “A” at position 132834960 on chromosome 6 referenced to B73AGPv04, preferably detectable by marker sequence of SEQ ID NO: 354,
lxxxiii. an “A” at position 132834951 on chromosome 6 referenced to B73AGPv04, preferably detectable by marker sequence of SEQ ID NO: 355,
lxxxiv. a “AC” at position 132834946 on chromosome 6 referenced to B73AGPv04, preferably detectable by marker sequence of SEQ ID NO: 356 and/or 357,
lxxxv. an “A” at position 132834942 on chromosome 6 referenced to B73AGPv04, preferably detectable by marker sequence of SEQ ID NO: 358,
lxxxvi. an “A” at position 132834929 on chromosome 6 referenced to B73AGPv04, preferably detectable by marker sequence of SEQ ID NO: 359,
lxxxvii. a “T” at position 132834927 on chromosome 6 referenced to B73AGPv04, preferably detectable by marker sequence of SEQ ID NO: 360,
lxxxviii. an “A” at position 132834924 on chromosome 6 referenced to B73AGPv04, preferably detectable by marker sequence of SEQ ID NO: 361,
lxxxix. a “C” at position 132834919 on chromosome 6 referenced to B73AGPv04, preferably detectable by marker sequence of SEQ ID NO: 362,
xc. a “T” at position 132834908 on chromosome 6 referenced to B73AGPv04, preferably detectable by marker sequence of SEQ ID NO: 363,
xci. an “A” at position 132834899 on chromosome 6 referenced to B73AGPv04, preferably detectable by marker sequence of SEQ ID NO: 364,
xcii. a “C” at position 132834754 on chromosome 6 referenced to B73AGPv04, preferably detectable by marker sequence of SEQ ID NO: 365,
xciii. a “C” at position 132834753 on chromosome 6 referenced to B73AGPv04, preferably detectable by marker sequence of SEQ ID NO: 366,
xciv. a “C” at position 132834748 on chromosome 6 referenced to B73AGPv04, preferably detectable by marker sequence of SEQ ID NO: 367,
xcv. an “A” at position 132834721 on chromosome 6 referenced to B73AGPv04, preferably detectable by marker sequence of SEQ ID NO: 368,
xcvi. an “A” at position 132834632 on chromosome 6 referenced to B73AGPv04, preferably detectable by marker sequence of SEQ ID NO: 369,
xcvii. a “C” at position 132834622 on chromosome 6 referenced to B73AGPv04, preferably detectable by marker sequence of SEQ ID NO: 370,
xcviii. a “G” at position 132834615 on chromosome 6 referenced to B73AGPv04, preferably detectable by marker sequence of SEQ ID NO: 371,
xcix. a “C” at position 132834602 on chromosome 6 referenced to B73AGPv04,
c. a “G” at position 132834589 on chromosome 6 referenced to B73AGPv04, preferably detectable by marker sequence of SEQ ID NO: 372,
ci. a “T” at position 132834581 on chromosome 6 referenced to B73AGPv04, preferably detectable by marker sequence of SEQ ID NO: 373,
cii. an “A” at position 132834577 on chromosome 6 referenced to B73AGPv04, preferably detectable by marker sequence of SEQ ID NO: 374,
ciii. a “G” at position 132834569 on chromosome 6 referenced to B73AGPv04, preferably detectable by marker sequence of SEQ ID NO: 375,
civ. a “C” at position 132834539 on chromosome 6 referenced to B73AGPv04, preferably detectable by marker sequence of SEQ ID NO: 376,
cv. a “GAG” at position 132834532 on chromosome 6 referenced to B73AGPv04, preferably detectable by marker sequence of SEQ ID NO: 377 and/or 378,
cvi. a “T” at position 132834494 on chromosome 6 referenced to B73AGPv04, preferably detectable by marker sequence of SEQ ID NO: 379,
cvii. a “T” at position 132834431 on chromosome 6 referenced to B73AGPv04, preferably detectable by marker sequence of SEQ ID NO: 380,
cviii. a “G” at position 132834389 on chromosome 6 referenced to B73AGPv04, preferably detectable by marker sequence of SEQ ID NO: 381,
cix. a “T” at position 132834208 on chromosome 6 referenced to B73AGPv04, preferably detectable by marker sequence of SEQ ID NO: 382,
cx. a “T” at position 132831963 on chromosome 6 referenced to B73AGPv04, preferably detectable by marker sequence of SEQ ID NO: 383,
cxi. a “G” at position 132831924 on chromosome 6 referenced to B73AGPv04, preferably detectable by marker sequence of SEQ ID NO: 384,
cxii. an “A” at position 132831915 on chromosome 6 referenced to B73AGPv04, preferably detectable by marker sequence of SEQ ID NO: 385,
cxiii. a “G” at position 132831839 on chromosome 6 referenced to B73AGPv04, preferably detectable by marker sequence of SEQ ID NO: 386,
cxiv. a “T” at position 132829096 on chromosome 6 referenced to B73AGPv04, preferably detectable by marker sequence of SEQ ID NO: 387,
cxv. an “A” at position 132828997 on chromosome 6 referenced to B73AGPv04, preferably detectable by marker sequence of SEQ ID NO: 388,
cxvi. a “G” at position 132828958 on chromosome 6 referenced to B73AGPv04, preferably detectable by marker sequence of SEQ ID NO: 389,
cxvii. a “G” at position 132828950 on chromosome 6 referenced to B73AGPv04, preferably detectable by marker sequence of SEQ ID NO: 390,
cxviii. a “C” at position 132828867 on chromosome 6 referenced to B73AGPv04, preferably detectable by marker sequence of SEQ ID NO: 391,
cxix. an “A” at position 132827606 on chromosome 6 referenced to B73AGPv04, preferably detectable by marker sequence of SEQ ID NO: 392,
cxx. an insertion of “AGTTCATAATAAAGTGATAGAGTT” (SEQ ID NO: 414) at position 132827596 on chromosome 6 referenced to B73AGPv04, preferably detectable by marker sequence of SEQ ID NO: 393 and/or 394,
cxxi. a “T” at position 132827573 on chromosome 6 referenced to B73AGPv04, preferably detectable by marker sequence of SEQ ID NO: 395,
cxxii. a “G” at position 132827566 on chromosome 6 referenced to B73AGPv04, preferably detectable by marker sequence of SEQ ID NO: 396,
cxxiii. an “A” at position 132827549 on chromosome 6 referenced to B73AGPv04, preferably detectable by marker sequence of SEQ ID NO: 397,
cxxiv. a “TAT” at position 132827545 on chromosome 6 referenced to B73AGPv04, preferably detectable by marker sequence of SEQ ID NO: 398 and/or 399,
cxxv. a “C” at position 132827302 on chromosome 6 referenced to B73AGPv04, preferably detectable by marker sequence of SEQ ID NO: 400,
cxxvi. a “T” at position 132826983 on chromosome 6 referenced to B73AGPv04, preferably detectable by marker sequence of SEQ ID NO: 401,
cxxvii. a “T” at position 132826940 on chromosome 6 referenced to B73AGPv04, preferably detectable by marker sequence of SEQ ID NO: 402,
cxxviii. a “T” at position 1333040380 on chromosome 6 referenced to B73AGPv04, preferably detectable by marker sequence of SEQ ID NO: 403,
cxxix. a “C” at position 1333040382 on chromosome 6 referenced to B73AGPv04, preferably detectable by marker sequence of SEQ ID NO: 404, or
cxxx. a “T” at position 133040760 on chromosome 6 referenced to B73AGPv04, preferably detectable by marker sequence of SEQ ID NO: 405.

The first maize plant is crossed to a second maize plant. Progeny plants are evaluated for the above haplotype or at least one marker allele linked to and associated with a haplotype in the first maize plant crossed with the second maize plant. Progeny plants are selected for that possess the haplotype in the first maize plant.

In some embodiments, the first maize plant is obtained and comprises within its genome a haplotype comprising a “G” at C16759-001-K1 and optionally one or more of a “C” at C12305-001-K1, a “C” at C12307-001-K1, a “G” at C16760-001-K1 and an “A” at C12314-001-K1, and/or a haplotype comprising variant nucleotide polymorphisms recited in Table 13 and/or a haplotype comprising one or more of
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50, and
x. an “A” at position 3342 referenced to SEQ ID NO: 50.

Also provided is a maize plant selected according to any of the above methods.

Modification of Plants with Site-Directed Nucleases

The disclosure provides a method for manufacturing a maize plant having anthracnose stalk rot resistance, comprising:
(a) introducing or introgressing into at least one cell of a maize plant any of the nucleic acid molecules described herein, or any of the expression cassettes described herein; or
(b.1) introducing of a site-directed nuclease and a repair matrix into at least one cell of a maize plant, wherein the site-directed nuclease is able to generate at least one double-strand break of the DNA in the genome of the at least one cell and the repair matrix comprises a nucleic acid molecule and/or a nucleic acid sequence of interest described herein; and
(b.2) cultivation of the at least one cell of (b.1) under conditions that allow a homology-directed repair or a homologous recombination, wherein the nucleic acid molecule is integrated from the repair matrix into the genome of the maize plant; or
(c.1) introducing of a site-directed nuclease, wherein the site-directed nuclease is able to generate at least one single-strand break of the DNA in the genome of the at least one cell and optionally the repair matrix comprises a nucleic acid molecule and/or a nucleic acid sequence of interest described herein; and
(c.2) cultivation of the at least one cell of (c.1) under conditions that allow introduction of at least one single nucleotide polymorphism of the anthracnose stalk rot resistance conferring genes of the present disclosure described herein or that allow a homology-directed repair or a homologous recombination, wherein the nucleic acid molecule is integrated from the repair matrix into the genome of the maize plant; and
(d) obtaining the plant having anthracnose stalk rot resistance from the at least one cell.

In various embodiments, the site-specific nuclease comprises a zinc-finger nuclease, a transcription activator-like effector nuclease, a CRISPR/Cas system, including a CRISPR/Cas9 system, a CRISPR/Cpf1 system, a CRISPR/MAD7, a CRISPR/CasX system, a CRISPR/CasY system, a Prime Editing system, a CRISPR-based base editor system, an engineered homing endonuclease, and a meganuclease, and/or any combination, variant, or catalytically active fragment thereof. The site-specific nuclease as used herein may include also mutant variants having nickase activity like nCas9 and/or non-functionality as nuclease like dCas9.

A CRISPR system, e.g., CRISPR/Cas, CRISPR/Cas9, CRISPR/Cpf1, a CRISPR/MAD7, CRISPR/CasX, or CRISPR/CasY, in its natural environment describes a molecular complex comprising at least one small and individual non-coding RNA in combination with a Cas nuclease or another CRISPR nuclease like a Cpf1 nuclease which can produce a specific DNA double-stranded break. Presently, CRISPR systems are categorized into 2 classes comprising five types of CRISPR systems, the type II system, for instance, using Cas9 as effector and the type V system using Cpf1 as effector molecule. In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems, for example, can code for a Cas9 nuclease as key enzyme for the interference step, which system contains both a crRNA and also a trans-activating RNA (tracrRNA) as the guide motif. These hybridize and form double-stranded (ds) RNA regions which are recognized by RNAseIII and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease. As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337: 816-821, incorporated by reference herein in its entirety. The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (International Patent Publication No. WO 2016/021973 A1, incorporated by reference herein in its entirety). For Cpf1 nucleases, the Cpf1-crRNA complex, without a tracrRNA, efficiently recognize and cleave target DNA proceeded by a short T-rich PAM in contrast to the commonly G-rich PAMs recognized by Cas9 systems. Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241, incorporated by reference herein in its entirety).

Presently, for example, Type II systems relying on Cas9, or a variant or any chimeric form thereof, as endonuclease have been modified for genome engineering. Synthetic CRISPR systems consisting of two components, a guide RNA (gRNA) also called single guide RNA (sgRNA) and a non-specific CRISPR-associated endonuclease can be used to generate knock-out cells or animals by co-expressing a gRNA specific to the gene to be targeted and capable of association with the endonuclease Cas9. Notably, the gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA (as “single guide RNA” (sgRNA) or simply “gRNA”). The genomic target can be any ˜20 nucleotide DNA sequence, provided that the target is present immediately upstream of a PAM sequence. The PAM sequence is of outstanding importance for target binding and the exact sequence is dependent upon the species of Cas9 and, for example, reads 5′-NGG-3′ or 5′ NAG 3′ (Standard IUPAC nucleotide code) (Jinek et al., Science 2012, supra) for a Streptococcus pyogenes derived Cas9. The PAM sequence for Cas9 from Staphylococcus aureus is NNGRRT or NNGRR(N). Many further variant CRISPR/Cas9 systems are known, including inter alia, Neisseria meningitidis Cas9 cleaving the PAM sequence NNNNGATT. A Streptococcus thermophilus Cas9 cleaving the PAM sequence NNAGAAW. Using modified Cas nucleases, targeted single-strand breaks can be introduced into a target sequence of interest. By the combined use of such a Cas nickase with different recombinant gRNAs highly site specific DNA double-strand breaks can be introduced using a double nicking system. Using one or more gRNAs can further increase the overall specificity and reduce off-target effects.

Once expressed, the Cas9 protein and the gRNA form a ribonucleoprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

CRISPR/Cas, e.g. CRISPR/Cas9, and likewise CRISPR/Cpf1 or CRISPR/CasX or CRISPR/CasY and other CRISPR systems are highly specific when gRNAs are designed correctly, but especially specificity is still a major concern, particularly for clinical uses or targeted plant GE based on the CRISPR technology. The specificity of the CRISPR system is determined in large part by how specific the gRNA targeting sequence is for the genomic target compared to the rest of the genome. Therefore, the methods according to the present disclosure when combined with the use of at least one CRISPR nuclease as site-specific nuclease and further combined with the use of a suitable CRISPR nucleic acid can provide a significantly more predictable outcome of GE. Whereas the CRISPR complex can mediate a highly precise cut of a genome or genetic material of a cell or cellular system at a specific site, the methods presented herein provide an additional control mechanism guaranteeing a programmable and predictable repair mechanism.

Covalent and non-covalent association or attachment can also apply for CRISPR nucleic acids sequences, which may comprise more than one portion, for example, a crRNA and a tracrRNA portion, which may be associated with each other as detailed above. In one embodiment, a repair template (repair matrix) nucleic acid sequence may be placed within a CRISPR nucleic acid sequence of interest to form a hybrid nucleic acid sequence according to the present disclosure, which hybrid may be formed by covalent and non-covalent association.

In various embodiments, the site-specific nuclease comprises a transcription activator-like effectors nuclease (TALEN). A TALEN can comprise a transcription activator-like effector (TALEs) from the bacterial genus Xanthomonas fused to a catalytic domain of a nuclease (e.g., FokI or a variant thereof). The DNA binding specificity of the TALE can be defined by repeat-variable di-residues (RVDs) of tandem-arranged 34/35-amino acid repeat units, such that one RVD specifically recognizes one nucleotide in the target DNA. The repeat units can be assembled to recognize basically any target sequences and fused to a catalytic domain of a nuclease create sequence specific endonucleases (see, e.g., Boch et al. (2009). Breaking the code of DNA binding specificity of TAL-type III effectors. Science, 326(5959), 1509-1512; Moscou & Bogdanove (2009). A simple cipher governs DNA recognition by TAL effectors. Science, 326(5959), 1501-1501; and WO 2010/079430, WO 2011/072246, WO 2011/154393, WO 2011/146121, WO 2012/001527, WO 2012/093833, WO 2012/104729, WO 2012/138927, WO 2012/138939). WO 2012/138927 further describes monomeric (compact) TALENs and TALEs with various catalytic domains and combinations thereof.

The nucleic acid molecule to be introduced into the maize plant may comprise a homology sequence. The homology sequence may be physically associated with the at least one nucleic acid sequence of interest within the nucleic acid molecule. As such, the homology sequence may be part of the at least one nucleic acid sequence of interest to be introduced. The homology sequence may be positioned within the 5′ and/or 3′ position of the at least one nucleic acid sequence of interest.

The homology sequence may comprise a sequence that has at least 85%-100% complementarity to one or more nucleic acid sequence(s) adjacent to the predetermined location on the genome to which the sequence is to be introduced, upstream and/or downstream from the predetermined location, over the entire length of the respective adjacent region(s). For high precision more than 95% homology/complementarity can be used to achieve a highly targeted repair event. As shown in Rubnitz et al., Mol. Cell Biol., 1984, 4(11), 2253-2258, also very low sequence homology might suffice to obtain a homologous recombination. As it is known to the skilled person, the degree of complementarity will depend on the genetic material to be modified, the nature of the planned edit, the complexity and size of a genome, the number of potential off-target sites, the genetic background and the environment within a cell or cellular system to be modified. In certain embodiments, the homology sequence comprises at least one spacer nucleotide. Alternatively, the spacer nucleotide can be present within the at least one nucleic acid sequence of interest to be introduced.

Without wishing to be bound by any theory, the homology sequence(s) can serve as templates to mediate homology-directed repair by having complementarity to at least one region, the upstream and/or the downstream region, adjacent to the predetermined location within the genetic material of the cellular system to be modified. The at least one nucleic acid sequence of interest and the flanking one or more homology region(s) thus can have the function of a repair template (RT) nucleic acid sequence. In certain embodiments, the RT may be further associated with another DNA and/or RNA sequence as mediated by complementary base pairing. In an alternative embodiment the RT may be associated with other sequence, for example, sequences of a vector, e.g., a plasmid vector, which vector can be used to amplify the RT prior to transformation.

Furthermore, the RT may also be physically associated with at least part of an amino acid component, preferably a site-specific nuclease. This configuration and association allows the availability of the RT in close physical proximity to the site of a double strand break (DSB), i.e., exactly at the position a targeted GE event is to be effected to allow even higher efficiency rates.

For example, the at least one RT may also be associated with at least one gRNA interacting with the at least one RT and further interacting with at least one portion of a CRISPR nuclease as site-specific nuclease.

A base editor enzyme or base editor or base editor system as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Preferably, the at least one base editor according to the present disclosure is temporarily or permanently linked to at least one site-specific effector, or optionally to a component of at least one site-specific effector complex. The linkage can be covalent and/or non-covalent.

Any base editor or site-specific effector, or a catalytically active fragment thereof, or any component of a base editor complex or of a site-specific effector complex as disclosed herein can be introduced into a cell as a nucleic acid fragment, the nucleic acid fragment representing or encoding a DNA, RNA or protein effector, or it can be introduced as DNA, RNA and/or protein, or any combination thereof. The nucleic acid sequence of interest may comprise any sequence from an anthracnose stalk rot resistance allele sequence described herein. The nucleic acid sequence of interest to be introduced or integrated into the plant genome may comprise for instances the entirety of the nucleic acid sequence of SEQ ID NO: 272, or a portion of the nucleic acid sequence of SEQ ID NO: 272, wherein the nucleic acid sequence of interest comprises one or more of the following polymorphisms:
an “A” at position 132836954 on chromosome 6 referenced to B73AGPv04, preferably detectable by marker sequence of SEQ ID NO: 273,
a “T” at position 132836944 on chromosome 6 referenced to B73AGPv04, preferably detectable by marker sequence of SEQ ID NO: 274,
an “A” at position 132836869 on chromosome 6 referenced to B73AGPv04, preferably detectable by marker sequence of SEQ ID NO: 275,
a “T” at position 132836849 on chromosome 6 referenced to B73AGPv04, preferably detectable by marker sequence of SEQ ID NO: 276,
a “T” at position 132836845 on chromosome 6 referenced to B73AGPv04, preferably detectable by marker sequence of SEQ ID NO: 277,
a “G” at position 132836840 on chromosome 6 referenced to B73AGPv04, preferably detectable by marker sequence of SEQ ID NO: 278,
a “T” at position 132836830 on chromosome 6 referenced to B73AGPv04, preferably detectable by marker sequence of SEQ ID NO: 279,
an “A” at position 132836810 on chromosome 6 referenced to B73AGPv04, preferably detectable by marker sequence of SEQ ID NO: 280,
a “CA” at position 132836805 on chromosome 6 referenced to B73AGPv04, preferably detectable by marker sequence of SEQ ID NO: 281 and/or 282,
a deletion of “ATC” at position 132836802 on chromosome 6 referenced to B73AGPv04, preferably detectable by marker sequence of SEQ ID NO: 283 and/or 284,
a deletion of “ATT” at position 132836799 on chromosome 6 referenced to B73AGPv04, preferably detectable by marker sequence of SEQ ID NO: 283 and/or 284,
an “A” at position 132836791 on chromosome 6 referenced to B73AGPv04, preferably detectable by marker sequence of SEQ ID NO: 285,
a “CTG” at position 132836787 on chromosome 6 referenced to B73AGPv04, preferably detectable by marker sequence of SEQ ID NO: 286 and/or 287,
a “GA” at position 132836781 on chromosome 6 referenced to B73AGPv04, preferably detectable by marker sequence of SEQ ID NO: 288 and/or 289,
a “T” at position 132836776 on chromosome 6 referenced to B73AGPv04, preferably detectable by marker sequence of SEQ ID NO: 290,
a “AG” at position 132836767 on chromosome 6 referenced to B73AGPv04, preferably detectable by marker sequence of SEQ ID NO: 291 and/or 292,
an “A” at position 132836762 on chromosome 6 referenced to B73AGPv04, preferably detectable by marker sequence of SEQ ID NO: 293,
an insertion of “CGCCAA” and an “A” at position 132836679 on chromosome 6 referenced to B73AGPv04, preferably detectable by marker sequence of SEQ ID NO: 294 and/or 295,
an “A” at position 132836678 on chromosome 6 referenced to B73AGPv04, preferably detectable by marker sequence of SEQ ID NO: 296,
a “G” at position 132836670 on chromosome 6 referenced to B73AGPv04, preferably detectable by marker sequence of SEQ ID NO: 297,
a “T” at position 132836667 on chromosome 6 referenced to B73AGPv04, preferably detectable by marker sequence of SEQ ID NO: 298,
an “A” at position 132836665 on chromosome 6 referenced to B73AGPv04, preferably detectable by marker sequence of SEQ ID NO: 299,
a “C” at position 132836662 on chromosome 6 referenced to B73AGPv04, preferably detectable by marker sequence of SEQ ID NO: 300,
a “T” at position 132836660 on chromosome 6 referenced to B73AGPv04, preferably detectable by marker sequence of SEQ ID NO: 301,
a “G” at position 132836651 on chromosome 6 referenced to B73AGPv04, preferably detectable by marker sequence of SEQ ID NO: 302,
a deletion of “AAT” at position 132836643 on chromosome 6 referenced to B73AGPv04, preferably detectable by marker sequence of SEQ ID NO: 303 and/or 304,
a deletion of “GCCATG” at position 132836636 on chromosome 6 referenced to B73AGPv04, preferably detectable by marker sequence of SEQ ID NO: 305 and/or 306,
a “AG” at position 132836622 on chromosome 6 referenced to B73AGPv04, preferably detectable by marker sequence of SEQ ID NO: 307 and/or 308,
a “AC” at position 132836620 on chromosome 6 referenced to B73AGPv04, preferably detectable by marker sequence of SEQ ID NO: 309 and/or 310,
a “G” at position 132836612 on chromosome 6 referenced to B73AGPv04, preferably detectable by marker sequence of SEQ ID NO: 311,
a “G” at position 132836603 on chromosome 6 referenced to B73AGPv04, preferably detectable by marker sequence of SEQ ID NO: 312,
a “C” at position 132836591 on chromosome 6 referenced to B73AGPv04, preferably detectable by marker sequence of SEQ ID NO: 313,
an “A” at position 132836586 on chromosome 6 referenced to B73AGPv04, preferably detectable by marker sequence of SEQ ID NO: 314,
a “G” at position 132836580 on chromosome 6 referenced to B73AGPv04, preferably detectable by marker sequence of SEQ ID NO: 315,
an “A” at position 132836069 on chromosome 6 referenced to B73AGPv04, preferably detectable by marker sequence of SEQ ID NO: 316,
an “A” at position 132836063 on chromosome 6 referenced to B73AGPv04, preferably detectable by marker sequence of SEQ ID NO: 317,
a “G” at position 132836061 on chromosome 6 referenced to B73AGPv04, preferably detectable by marker sequence of SEQ ID NO: 318,
a “G” at position 132836056 on chromosome 6 referenced to B73AGPv04, preferably detectable by marker sequence of SEQ ID NO: 319,
a “C” at position 132836050 on chromosome 6 referenced to B73AGPv04, preferably detectable by marker sequence of SEQ ID NO: 320,
a “CGT” at position 132836047 on chromosome 6 referenced to B73AGPv04, preferably detectable by marker sequence of SEQ ID NO: 321 and/or 322,
a “G” at position 132836034 on chromosome 6 referenced to B73AGPv04, preferably detectable by marker sequence of SEQ ID NO: 323,
a “G” at position 132836031 on chromosome 6 referenced to B73AGPv04, preferably detectable by marker sequence of SEQ ID NO: 324,
a “T” at position 132836019 on chromosome 6 referenced to B73AGPv04, preferably detectable by marker sequence of SEQ ID NO: 325,
a “G” at position 132836008 on chromosome 6 referenced to B73AGPv04, preferably detectable by marker sequence of SEQ ID NO: 326,
a “G” at position 132835978 on chromosome 6 referenced to B73AGPv04, preferably detectable by marker sequence of SEQ ID NO: 327,
a “G” at position 132835910 on chromosome 6 referenced to B73AGPv04, preferably detectable by marker sequence of SEQ ID NO: 328,
a “G” at position 132835851 on chromosome 6 referenced to B73AGPv04, preferably detectable by marker sequence of SEQ ID NO: 329,
a “C” at position 132835819 on chromosome 6 referenced to B73AGPv04, preferably detectable by marker sequence of SEQ ID NO: 330,
a “G” at position 132835803 on chromosome 6 referenced to B73AGPv04, preferably detectable by marker sequence of SEQ ID NO: 331,
a “T” at position 132835793 on chromosome 6 referenced to B73AGPv04, preferably detectable by marker sequence of SEQ ID NO: 332,
a “C” at position 132835788 on chromosome 6 referenced to B73AGPv04, preferably detectable by marker sequence of SEQ ID NO: 333,
an “A” at position 132835781 on chromosome 6 referenced to B73AGPv04, preferably detectable by marker sequence of SEQ ID NO: 334,
an “A” at position 132835779 on chromosome 6 referenced to B73AGPv04, preferably detectable by marker sequence of SEQ ID NO: 335,
a “T” at position 132835776 on chromosome 6 referenced to B73AGPv04, preferably detectable by marker sequence of SEQ ID NO: 336,
a “G” at position 132835746 on chromosome 6 referenced to B73AGPv04, preferably detectable by marker sequence of SEQ ID NO: 337,
an “A” at position 132835729 on chromosome 6 referenced to B73AGPv04, preferably detectable by marker sequence of SEQ ID NO: 338,
a “C” at position 132835728 on chromosome 6 referenced to B73AGPv04, preferably detectable by marker sequence of SEQ ID NO: 339,
an insertion of “GACATC” at position 132835722 on chromosome 6 referenced to B73AGPv04, preferably detectable by marker sequence of SEQ ID NO: 340 and/or 341,
a “T” at position 132835713 on chromosome 6 referenced to B73AGPv04, preferably detectable by marker sequence of SEQ ID NO: 342,
a deletion of “GAA” at position 132835708 on chromosome 6 referenced to B73AGPv04, preferably detectable by marker sequence of SEQ ID NO: 343 and/or 344,
a deletion of “ATA” at position 132835705 on chromosome 6 referenced to B73AGPv04, preferably detectable by marker sequence of SEQ ID NO: 343 and/or 344,
a deletion of “GAT” at position 132835702 on chromosome 6 referenced to B73AGPv04, preferably detectable by marker sequence of SEQ ID NO: 343 and/or 344,
a “G” at position 132835693 on chromosome 6 referenced to B73AGPv04, preferably detectable by marker sequence of SEQ ID NO: 345,
a “T” at position 132835681 on chromosome 6 referenced to B73AGPv04, preferably detectable by marker sequence of SEQ ID NO: 346,
a “C” at position 132835671 on chromosome 6 referenced to B73AGPv04, preferably detectable by marker sequence of SEQ ID NO: 347,
a “T” at position 132835666 on chromosome 6 referenced to B73AGPv04, preferably detectable by marker sequence of SEQ ID NO: 348,
an “A” at position 132835271 on chromosome 6 referenced to B73AGPv04, preferably detectable by marker sequence of SEQ ID NO: 349,
a “GG” at position 132835267 on chromosome 6 referenced to B73AGPv04, preferably detectable by marker sequence of SEQ ID NO: 350 and/or 351,
a “T” at position 132835147 on chromosome 6 referenced to B73AGPv04, preferably detectable by marker sequence of SEQ ID NO: 352,
a “C” at position 132835031 on chromosome 6 referenced to B73AGPv04, preferably detectable by marker sequence of SEQ ID NO: 353,
an “A” at position 132834960 on chromosome 6 referenced to B73AGPv04, preferably detectable by marker sequence of SEQ ID NO: 354,
an “A” at position 132834951 on chromosome 6 referenced to B73AGPv04, preferably detectable by marker sequence of SEQ ID NO: 355,
a “AC” at position 132834946 on chromosome 6 referenced to B73AGPv04, preferably detectable by marker sequence of SEQ ID NO: 356 and/or 357,
an “A” at position 132834942 on chromosome 6 referenced to B73AGPv04, preferably detectable by marker sequence of SEQ ID NO: 358,
an “A” at position 132834929 on chromosome 6 referenced to B73AGPv04, preferably detectable by marker sequence of SEQ ID NO: 359,
a “T” at position 132834927 on chromosome 6 referenced to B73AGPv04, preferably detectable by marker sequence of SEQ ID NO: 360,
an “A” at position 132834924 on chromosome 6 referenced to B73AGPv04, preferably detectable by marker sequence of SEQ ID NO: 361,
a “C” at position 132834919 on chromosome 6 referenced to B73AGPv04, preferably detectable by marker sequence of SEQ ID NO: 362,
a “T” at position 132834908 on chromosome 6 referenced to B73AGPv04, preferably detectable by marker sequence of SEQ ID NO: 363,
an “A” at position 132834899 on chromosome 6 referenced to B73AGPv04, preferably detectable by marker sequence of SEQ ID NO: 364,
a “C” at position 132834754 on chromosome 6 referenced to B73AGPv04, preferably detectable by marker sequence of SEQ ID NO: 365,
a “C” at position 132834753 on chromosome 6 referenced to B73AGPv04, preferably detectable by marker sequence of SEQ ID NO: 366,
a “C” at position 132834748 on chromosome 6 referenced to B73AGPv04, preferably detectable by marker sequence of SEQ ID NO: 367,
an “A” at position 132834721 on chromosome 6 referenced to B73AGPv04, preferably detectable by marker sequence of SEQ ID NO: 368,
an “A” at position 132834632 on chromosome 6 referenced to B73AGPv04, preferably detectable by marker sequence of SEQ ID NO: 369,
a “C” at position 132834622 on chromosome 6 referenced to B73AGPv04, preferably detectable by marker sequence of SEQ ID NO: 370,
a “G” at position 132834615 on chromosome 6 referenced to B73AGPv04, preferably detectable by marker sequence of SEQ ID NO: 371,
a “C” at position 132834602 on chromosome 6 referenced to B73AGPv04,
a “G” at position 132834589 on chromosome 6 referenced to B73AGPv04, preferably detectable by marker sequence of SEQ ID NO: 372,
a “T” at position 132834581 on chromosome 6 referenced to B73AGPv04, preferably detectable by marker sequence of SEQ ID NO: 373,
an “A” at position 132834577 on chromosome 6 referenced to B73AGPv04, preferably detectable by marker sequence of SEQ ID NO: 374,
a “G” at position 132834569 on chromosome 6 referenced to B73AGPv04, preferably detectable by marker sequence of SEQ ID NO: 375,
a “C” at position 132834539 on chromosome 6 referenced to B73AGPv04, preferably detectable by marker sequence of SEQ ID NO: 376,
a “GAG” at position 132834532 on chromosome 6 referenced to B73AGPv04, preferably detectable by marker sequence of SEQ ID NO: 377 and/or 378,
a “T” at position 132834494 on chromosome 6 referenced to B73AGPv04, preferably detectable by marker sequence of SEQ ID NO: 379,
a “T” at position 132834431 on chromosome 6 referenced to B73AGPv04, preferably detectable by marker sequence of SEQ ID NO: 380,
a “G” at position 132834389 on chromosome 6 referenced to B73AGPv04, preferably detectable by marker sequence of SEQ ID NO: 381,
a “T” at position 132834208 on chromosome 6 referenced to B73AGPv04, preferably detectable by marker sequence of SEQ ID NO: 382,
a “T” at position 132831963 on chromosome 6 referenced to B73AGPv04, preferably detectable by marker sequence of SEQ ID NO: 383,
a “G” at position 132831924 on chromosome 6 referenced to B73AGPv04, preferably detectable by marker sequence of SEQ ID NO: 384,
an “A” at position 132831915 on chromosome 6 referenced to B73AGPv04, preferably detectable by marker sequence of SEQ ID NO: 385,
a “G” at position 132831839 on chromosome 6 referenced to B73AGPv04, preferably detectable by marker sequence of SEQ ID NO: 386,
a “T” at position 132829096 on chromosome 6 referenced to B73AGPv04, preferably detectable by marker sequence of SEQ ID NO: 387,
an “A” at position 132828997 on chromosome 6 referenced to B73AGPv04, preferably detectable by marker sequence of SEQ ID NO: 388,
a “G” at position 132828958 on chromosome 6 referenced to B73AGPv04, preferably detectable by marker sequence of SEQ ID NO: 389,
a “G” at position 132828950 on chromosome 6 referenced to B73AGPv04, preferably detectable by marker sequence of SEQ ID NO: 390,
a “C” at position 132828867 on chromosome 6 referenced to B73AGPv04, preferably detectable by marker sequence of SEQ ID NO: 391,
an “A” at position 132827606 on chromosome 6 referenced to B73AGPv04, preferably detectable by marker sequence of SEQ ID NO: 392,
an insertion of “AGTTCATAATAAAGTGATAGAGTT” (SEQ ID NO: 414) at position 132827596 on chromosome 6 referenced to B73AGPv04, preferably detectable by marker sequence of SEQ ID NO: 393 and/or 394,
a “T” at position 132827573 on chromosome 6 referenced to B73AGPv04, preferably detectable by marker sequence of SEQ ID NO: 395,
a “G” at position 132827566 on chromosome 6 referenced to B73AGPv04, preferably detectable by marker sequence of SEQ ID NO: 396,
an “A” at position 132827549 on chromosome 6 referenced to B73AGPv04, preferably detectable by marker sequence of SEQ ID NO: 397,
a “TAT” at position 132827545 on chromosome 6 referenced to B73AGPv04, preferably detectable by marker sequence of SEQ ID NO: 398 and/or 399,
a “C” at position 132827302 on chromosome 6 referenced to B73AGPv04, preferably detectable by marker sequence of SEQ ID NO: 400,
a “T” at position 132826983 on chromosome 6 referenced to B73AGPv04, preferably detectable by marker sequence of SEQ ID NO: 401,
a “T” at position 132826940 on chromosome 6 referenced to B73AGPv04, preferably detectable by marker sequence of SEQ ID NO: 402,
a “T” at position 1333040380 on chromosome 6 referenced to B73AGPv04, preferably detectable by marker sequence of SEQ ID NO: 403,
a “C” at position 1333040382 on chromosome 6 referenced to B73AGPv04, preferably detectable by marker sequence of SEQ ID NO: 404, or
a “T” at position 133040760 on chromosome 6 referenced to B73AGPv04, preferably detectable by marker sequence of SEQ ID NO: 405.

The nucleic acid sequence of interest to be introduced or integrated into the plant genome may comprise additionally or alternatively the entirety of the nucleic acid sequence of SEQ ID NO: 50, or a portion of the nucleic acid sequence of SEQ ID NO: 50, wherein the nucleic acid sequence of interest comprises one or more of the following polymorphisms:
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50, and
x. an “A” at position 3342 referenced to SEQ ID NO: 50.

In certain embodiments, the nucleic acid sequence of interest is operatively linked to a heterologous regulatory element, preferably to a heterologous promoter. In certain embodiments, the nucleic acid sequence of interest may comprise an expression cassette comprising any of the above nucleotide sequences.

The method may be effective to introduce, or integrate, the sequence of interest into chromosome 4 or 6 of the cell of the maize plant. Introduction of the sequence of interest may be sufficient to confer to a maize plant cultivated from the modified maize cell a resistance to a fungal pathogen, e.g., a fungal pathogen causing anthracnose stalk rot. To detect whether the sequence of interest has been integrated into chromosome 4 or 6 of the cell, PCR amplification of genomic DNA from the cell or its progeny may be performed using primers. In some embodiments, the primers detect a variant nucleotide of Table 13. In some embodiments, the primers detect a variant nucleotide selected from any of SEQ ID NOS: 58-78. In some embodiments, the nucleic acid sequence of interest does not produce the amplicon according to SEQ ID NO: 119 (Amplicon 7) upon polymerase chain reaction amplification using primers of SEQ ID NOs: 120 and 121, primers of SEQ ID NOs: 122 and 123, or primers of SEQ ID NOs: 124 and 125. In some embodiments, the nucleic acid sequence of interest does not produce an amplicon selected from the group consisting of SEQ ID NOs: 94, 101, 106, 109, and 114 upon polymerase chain reaction amplification by means of primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118. In certain embodiments, the nucleic acid sequence of interest is operatively linked to a heterologous regulatory element, preferably to a heterologous promoter. In certain embodiments, the nucleic acid sequence of interest may comprise an expression cassette comprising any of the above nucleotide sequences.

The nucleic acid sequence of interest may comprise one or more nucleotide sequences selected from the group consisting of:
i. a nucleotide sequence of SEQ ID NO: 209, 212, 215, 218, 221, 224, 227, 230, 233, 236, 239, 242, 245, 248, 251, 254, 257, 260, 263, 266 or 269, preferably SEQ ID NO: 266 or 269,
ii. a nucleotide sequence having a coding sequence of SEQ ID NO: 210, 213, 216, 219, 222, 225, 228, 231, 234, 240, 243, 246, 249, 252, 255, 258, 261, 264, 267 or 270, preferably SEQ ID NO: 267 or 270,
iii. a nucleotide sequence which is complementary to a sequence from i. or ii.,
iv. a nucleotide sequence with at least 80% or 85%, preferably at least 90%, 91%, 92%, 93%, 94% or 95%, more preferably at least 96%, 97%, 98% or 99% identity to a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 211, 214, 217, 220, 223, 226, 229, 232, 235, 238, 241, 244, 247, 250, 253, 256, 259, 262, 265, 268 or 271, preferably SEQ ID NO: 268 or 271, or
viii. a nucleotide sequence which encodes for a protein comprising a sequence with at least 80% or 85%, preferably at least 90%, 91%, 92%, 93%, 94% or 95%, more preferably at least 96%, 97%, 98% or 99% identity to the sequence of SEQ ID NO: 211, 214, 217, 220, 223, 226, 229, 232, 235, 238, 241, 244, 247, 250, 253, 256, 259, 262, 265, 268 or 271, preferably SEQ ID NO: 268 or 271.

In certain embodiments, the nucleic acid sequence of interest is operatively linked to a heterologous regulatory element, preferably to a heterologous promoter. In certain embodiments, the nucleic acid sequence of interest may comprise an expression cassette comprising any of the above nucleotide sequences i) through viii). Introduction of the sequence of interest may be sufficient to confer to a maize plant cultivated from the modified maize cell a resistance to a fungal pathogen, e.g., a fungal pathogen causing anthracnose stalk rot.

The nucleic acid sequence of interest may comprise a nucleotide sequence encoding for a protein comprising the sequence of any one of SEQ ID NO: 211, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of any one of SEQ ID NO: 211. Alternatively, the nucleic acid sequence of interest may comprise a nucleotide sequence encoding for a protein comprising the sequence of SEQ ID NO: 214, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 214. Alternatively, the nucleic acid sequence of interest may comprise a nucleotide sequence encoding for a protein comprising the sequence of SEQ ID NO: 217, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 217. Alternatively, the nucleic acid sequence of interest may comprise a nucleotide sequence encoding for a protein comprising the sequence of SEQ ID NO: 220, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 220. Alternatively, the nucleic acid sequence of interest may comprise a nucleotide sequence encoding for a protein comprising the sequence of SEQ ID NO: 223, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 223. Alternatively, the nucleic acid sequence of interest may comprise a nucleotide sequence encoding for a protein comprising the sequence of SEQ ID NO: 226, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 226. Alternatively, the nucleic acid sequence of interest may comprise a nucleotide sequence encoding for a protein comprising the sequence of SEQ ID NO: 229, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 229. Alternatively, the nucleic acid sequence of interest may comprise a nucleotide sequence encoding for a protein comprising the sequence of SEQ ID NO: 232, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 232. In certain embodiments, the nucleic acid sequence of interest may comprise an expression cassette comprising any of the above nucleotide sequences

The nucleic acid sequence of interest may comprise a nucleotide sequence encoding for a protein comprising the sequence of SEQ ID NO: 235, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 235. The nucleic acid sequence of interest may comprise a nucleotide sequence encoding for a protein comprising the sequence of SEQ ID NO: 238, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 238. The nucleic acid sequence of interest may comprise a nucleotide sequence encoding for a protein comprising the sequence of SEQ ID NO: 241, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 241. The nucleic acid sequence of interest may comprise a nucleotide sequence encoding for a protein comprising the sequence of SEQ ID NO: 244, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 244. The nucleic acid sequence of interest may comprise a nucleotide sequence encoding for a protein comprising the sequence of SEQ ID NO: 247, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 247. The nucleic acid sequence of interest may comprise a nucleotide sequence encoding for a protein comprising the sequence of SEQ ID NO: 250, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 250. The nucleic acid sequence of interest may comprise a nucleotide sequence encoding for a protein comprising the sequence of SEQ ID NO: 253, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 253. The nucleic acid sequence of interest may comprise a nucleotide sequence encoding for a protein comprising the sequence of SEQ ID NO: 256, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 256. The nucleic acid sequence of interest may comprise a nucleotide sequence encoding for a protein comprising the sequence of SEQ ID NO: 259, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 259. Alternatively, the nucleic acid sequence of interest may comprise a nucleotide sequence encoding for a protein comprising the sequence of SEQ ID NO: 262, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 262. The nucleic acid sequence of interest may comprise a nucleotide sequence encoding for a protein comprising the sequence of SEQ ID NO: 265, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 265. Alternatively, the nucleic acid sequence of interest may comprise a nucleotide sequence encoding for a protein comprising the sequence of SEQ ID NO: 268, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 268. Alternatively, the nucleic acid sequence of interest may comprise a nucleotide sequence encoding for a protein comprising the sequence of SEQ ID NO: 271, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 271. In certain embodiments, the nucleic acid sequence of interest may comprise an expression cassette comprising any of the above nucleotide sequences.

The nucleic acid sequence of interest may comprise one or more nucleotide sequences selected from the group consisting of
i. a nucleotide sequence of SEQ ID NO: 1, 20, 23, 44 or 47,
ii. a nucleotide sequence which is complementary to a sequence from i.,
iii. a nucleotide sequence which has at least 80% or 85%, preferably at least 90%, 91%, 92%, 93%, 94% or 95%, more preferably at least 96%, 97%, 98% or 99% identity with a sequence from i. or ii.,
iv. a nucleotide sequence which differs from a nucleic acid sequence according to i., ii. or iii. depending on the degeneracy of the genetic code,
v. a nucleotide sequence which hybridizes with a nucleic acid sequence according to i., ii. or iii. under stringent conditions,
vi. a nucleotide sequence which encodes for a protein comprising the sequence of SEQ ID NO: 11-19, 22, 34-43, 46 or 49, or
vii. a nucleotide sequence which encodes for a protein which has at least 80% or 85%, preferably at least 90%, 91%, 92%, 93%, 94% or 95%, more preferably at least 960%, 97%, 98% or 99% identity with the sequence of SEQ ID NOs: 11-19, 22, 34-43, 46 or 49.

In certain embodiments, the nucleic acid sequence of interest is operatively linked to a heterologous regulatory element, preferably to a heterologous promoter. In certain embodiments, the nucleic acid sequence of interest may comprise an expression cassette comprising any of the above nucleic acids i) through vii). Introduction of the sequence of interest may be sufficient to confer to a maize plant cultivated from the modified maize cell a resistance to a fungal pathogen, e.g., a fungal pathogen causing anthracnose stalk rot.

The nucleic acid sequence of interest may comprise a nucleotide sequence encoding for a protein comprising the sequence of SEQ ID NO: 11, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 11. The nucleic acid sequence of interest may comprise a nucleotide sequence encoding for a protein comprising the sequence of SEQ ID NO: 12, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 12. The nucleic acid sequence of interest may comprise a nucleotide sequence encoding for a protein comprising the sequence of SEQ ID NO: 13, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 13. The nucleic acid sequence of interest may comprise a nucleotide sequence encoding for a protein comprising the sequence of SEQ ID NO: 14, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 14. The nucleic acid sequence of interest may comprise a nucleotide sequence encoding for a protein comprising the sequence of SEQ ID NO: 15, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 15. The nucleic acid sequence of interest may comprise a nucleotide sequence encoding for a protein comprising the sequence of SEQ ID NO: 16, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 16. The nucleic acid sequence of interest may comprise a nucleotide sequence encoding for a protein comprising the sequence of SEQ ID NO: 17, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 17. The nucleic acid sequence of interest may comprise a nucleotide sequence encoding for a protein comprising the sequence of SEQ ID NO: 18, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 18. The nucleic acid sequence of interest may comprise a nucleotide sequence encoding for a protein comprising the sequence of SEQ ID NO: 19, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 19.

The nucleic acid sequence of interest may comprise a nucleotide sequence encoding for a protein comprising the sequence of SEQ ID NO: 22, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 22. The nucleic acid sequence of interest may comprise a nucleotide sequence encoding for a protein comprising the sequence of SEQ ID NO: 34, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 34. The nucleic acid sequence of interest may comprise a nucleotide sequence encoding for a protein comprising the sequence of SEQ ID NO: 35, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 35. The nucleic acid sequence of interest may comprise a nucleotide sequence encoding for a protein comprising the sequence of SEQ ID NO: 36, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 36. The nucleic acid sequence of interest may comprise a nucleotide sequence encoding for a protein comprising the sequence of SEQ ID NO: 37, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 37. The nucleic acid sequence of interest may comprise a nucleotide sequence encoding for a protein comprising the sequence of SEQ ID NO: 38, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 38. The nucleic acid sequence of interest may comprise a nucleotide sequence encoding for a protein comprising the sequence of SEQ ID NO: 39, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 39. The nucleic acid sequence of interest may comprise a nucleotide sequence encoding for a protein comprising the sequence of SEQ ID NO: 40, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 40. The nucleic acid sequence of interest may comprise a nucleotide sequence encoding for a protein comprising the sequence of SEQ ID NO: 41, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 41. The nucleic acid sequence of interest may comprise a nucleotide sequence encoding for a protein comprising the sequence of SEQ ID NO: 42, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 42. The nucleic acid sequence of interest may comprise a nucleotide sequence encoding for a protein comprising the sequence of SEQ ID NO: 43, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 43. The nucleic acid sequence of interest may comprise a nucleotide sequence encoding for a protein comprising the sequence of SEQ ID NO: 46, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 46. The nucleic acid sequence of interest may comprise a nucleotide sequence encoding for a protein comprising the sequence of SEQ ID NO: 49, or a protein comprising a sequence at least 80%, 81%, 82%, 83%, 84%, 85%, 86%, 87%, 88%, 89%, 90%, 91%, 92%, 93%, 94%, 95%, 96%, 97%, 98%, or 99% identical to a protein comprising the sequence of SEQ ID NO: 49.

In various embodiments, the homology-directed repair is mediated by non-homologous end joining (NHEJ). Without wishing to be bound by theory, in eukaryotic cells, genome integrity is ensured by robust and partially redundant mechanisms for repairing DNA DSBs caused by environmental stresses and errors of cellular DNA processing machinery. In most eukaryotic cells and at most stages of the respective cell cycle, the non-homologous end-joining (NHEJ) DNA repair pathway is the highly dominant form of repair. A second pathway uses homologous recombination (HR) of similar DNA sequences to repair DSBs. This pathway can usually be used in the S and G2 stages of the cell cycle by templating from the duplicated homologous region of a paired chromosome to precisely repair the DSB. However, an artificially-provided repair template (RT) with homology to the target can also be used to repair the DSB, in a process known as homology-directed repair (HDR) or gene targeting. By this strategy it is possible to introduce very precise, targeted changes in the genomes of eukaryotic cells.

NHEJ is the dominant nuclear response in animals and plants which does not require homologous sequences, but is often error-prone and thus potentially mutagenic (Wyman C., Kanaar R. “DNA double-strand break repair: all's well that ends well”, Annu. Rev. Genet., 2006, 40, 363-83). Classical- and backup-NHEJ pathways are known relying on different mechanism, wherein both pathways are error-prone. Repair by HDR requires homology, but those HDR pathways that use an intact chromosome to repair the broken one, i.e. double-strand break repair and synthesis-dependent strand annealing, are highly accurate. In the classical DSB repair pathway, the 3′ ends invade an intact homologous template then serve as a primer for DNA repair synthesis, ultimately leading to the formation of double Holliday junctions (dHJs). dHJs are four-stranded branched structures that form when elongation of the invasive strand “captures” and synthesizes DNA from the second DSB end. The individual HJs are resolved via cleavage in one of two ways. Synthesis-dependent strand annealing is conservative, and results exclusively in non-crossover events. This means that all newly synthesized sequences are present on the same molecule. Unlike the NHEJ repair pathway, following strand invasion and D loop formation in synthesis-dependent strand annealing, the newly synthesized portion of the invasive strand is displaced from the template and returned to the processed end of the non-invading strand at the other DSB end. The 3′ end of the non-invasive strand is elongated and ligated to fill the gap. There is a further pathway of HDR, called break-induced repair pathway not yet fully characterized. A central feature of this pathway is the presence of only one invasive end at a DSB that can be used for repair.

In various embodiments, the homology-directed repair is mediated by microhomology-mediated end joining (MMEJ). Without wishing to be bound by theory, MMEJ has been recognized as a distinct type of DSB repair in eukaryotes. Only very short (2-14 bp) regions of homology are needed for this pathway, and it typically leaves deletions like single-strand annealing. MMEJ has also been distinguished genetically from the homologous recombination and NHEJ pathways and in mammalian cells acts as a backup to NHEJ (Kwon, T., Huq, E., & Herrin, D. L. (2010). Microhomology-mediated and nonhomologous repair of a double-strand break in the chloroplast genome of Arabidopsis. Proceedings of the National Academy of Sciences of the United States of America, 107(31), 13954-13959).

### Maize Plants Resistant to Stalk Rot

In one aspect is provided a maize plant comprising a resistance locus associated with anthracnose stalk rot resistance, wherein the resistance locus is located on chromosome 4, and where the resistance locus is introgressed into the maize plant according to any of the methods described herein. In various embodiments, the maize plant does not further comprise a resistance locus associated with anthracnose stalk rot resistance that is found on chromosome 6. In various embodiments, the maize plant only has one resistance locus associated with anthracnose stalk rot resistance that is located on chromosome 4.

In one aspect is provided a maize plant comprising a resistance locus associated with anthracnose stalk rot resistance, wherein the resistance locus is located on chromosome 6, and where the resistance locus is introgressed into the maize plant according to any of the methods described herein. The maize plant does not further comprise a resistance locus associated with anthracnose stalk rot resistance that is found on chromosome 4.

In one embodiment is provided a maize plant comprising a resistance locus on chromosome 4 associated with anthracnose stalk rot resistance, where the resistance locus located on chromosome 4 comprises a nucleic acid molecule encoding a Rcg1 resistance allele having a haplotype comprising one or more nucleotide polymorphism selected from the group consisting of:
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50, and
x. an “A” at position 3342 referenced to SEQ ID NO: 50.

In one embodiment is provided a maize plant comprising a resistance locus on chromosome 6 associated with anthracnose stalk rot resistance, where the resistance locus located on chromosome 6 comprises a nucleic acid molecule encoding an anthracnose stalk rot resistance allele having a haplotype comprising one or more nucleotide polymorphism selected from the group consisting of:
an “A” at position 132836954 on chromosome 6 referenced to B73AGPv04, preferably detectable by marker sequence of SEQ ID NO: 273,
a “T” at position 132836944 on chromosome 6 referenced to B73AGPv04, preferably detectable by marker sequence of SEQ ID NO: 274,
an “A” at position 132836869 on chromosome 6 referenced to B73AGPv04, preferably detectable by marker sequence of SEQ ID NO: 275,
a “T” at position 132836849 on chromosome 6 referenced to B73AGPv04, preferably detectable by marker sequence of SEQ ID NO: 276,
a “T” at position 132836845 on chromosome 6 referenced to B73AGPv04, preferably detectable by marker sequence of SEQ ID NO: 277,
a “G” at position 132836840 on chromosome 6 referenced to B73AGPv04, preferably detectable by marker sequence of SEQ ID NO: 278,
a “T” at position 132836830 on chromosome 6 referenced to B73AGPv04, preferably detectable by marker sequence of SEQ ID NO: 279,
an “A” at position 132836810 on chromosome 6 referenced to B73AGPv04, preferably detectable by marker sequence of SEQ ID NO: 280,
a “CA” at position 132836805 on chromosome 6 referenced to B73AGPv04, preferably detectable by marker sequence of SEQ ID NO: 281 and/or 282,
a deletion of “ATC” at position 132836802 on chromosome 6 referenced to B73AGPv04, preferably detectable by marker sequence of SEQ ID NO: 283 and/or 284,
a deletion of “ATT” at position 132836799 on chromosome 6 referenced to B73AGPv04, preferably detectable by marker sequence of SEQ ID NO: 283 and/or 284,
an “A” at position 132836791 on chromosome 6 referenced to B73AGPv04, preferably detectable by marker sequence of SEQ ID NO: 285,
a “CTG” at position 132836787 on chromosome 6 referenced to B73AGPv04, preferably detectable by marker sequence of SEQ ID NO: 286 and/or 287,
a “GA” at position 132836781 on chromosome 6 referenced to B73AGPv04, preferably detectable by marker sequence of SEQ ID NO: 288 and/or 289,
a “T” at position 132836776 on chromosome 6 referenced to B73AGPv04, preferably detectable by marker sequence of SEQ ID NO: 290,
a “AG” at position 132836767 on chromosome 6 referenced to B73AGPv04, preferably detectable by marker sequence of SEQ ID NO: 291 and/or 292,
an “A” at position 132836762 on chromosome 6 referenced to B73AGPv04, preferably detectable by marker sequence of SEQ ID NO: 293,
an insertion of “CGCCAA” and an “A” at position 132836679 on chromosome 6 referenced to B73AGPv04, preferably detectable by marker sequence of SEQ ID NO: 294 and/or 295,
an “A” at position 132836678 on chromosome 6 referenced to B73AGPv04, preferably detectable by marker sequence of SEQ ID NO: 296,
a “G” at position 132836670 on chromosome 6 referenced to B73AGPv04, preferably detectable by marker sequence of SEQ ID NO: 297,
a “T” at position 132836667 on chromosome 6 referenced to B73AGPv04, preferably detectable by marker sequence of SEQ ID NO: 298,
an “A” at position 132836665 on chromosome 6 referenced to B73AGPv04, preferably detectable by marker sequence of SEQ ID NO: 299,
a “C” at position 132836662 on chromosome 6 referenced to B73AGPv04, preferably detectable by marker sequence of SEQ ID NO: 300,
a “T” at position 132836660 on chromosome 6 referenced to B73AGPv04, preferably detectable by marker sequence of SEQ ID NO: 301,
a “G” at position 132836651 on chromosome 6 referenced to B73AGPv04, preferably detectable by marker sequence of SEQ ID NO: 302,
a deletion of “AAT” at position 132836643 on chromosome 6 referenced to B73AGPv04, preferably detectable by marker sequence of SEQ ID NO: 303 and/or 304,
a deletion of “GCCATG” at position 132836636 on chromosome 6 referenced to B73AGPv04, preferably detectable by marker sequence of SEQ ID NO: 305 and/or 306,
a “AG” at position 132836622 on chromosome 6 referenced to B73AGPv04, preferably detectable by marker sequence of SEQ ID NO: 307 and/or 308,
a “AC” at position 132836620 on chromosome 6 referenced to B73AGPv04, preferably detectable by marker sequence of SEQ ID NO: 309 and/or 310,
a “G” at position 132836612 on chromosome 6 referenced to B73AGPv04, preferably detectable by marker sequence of SEQ ID NO: 311,
a “G” at position 132836603 on chromosome 6 referenced to B73AGPv04, preferably detectable by marker sequence of SEQ ID NO: 312,
a “C” at position 132836591 on chromosome 6 referenced to B73AGPv04, preferably detectable by marker sequence of SEQ ID NO: 313,
an “A” at position 132836586 on chromosome 6 referenced to B73AGPv04, preferably detectable by marker sequence of SEQ ID NO: 314,
a “G” at position 132836580 on chromosome 6 referenced to B73AGPv04, preferably detectable by marker sequence of SEQ ID NO: 315,
an “A” at position 132836069 on chromosome 6 referenced to B73AGPv04, preferably detectable by marker sequence of SEQ ID NO: 316,
an “A” at position 132836063 on chromosome 6 referenced to B73AGPv04, preferably detectable by marker sequence of SEQ ID NO: 317,
a “G” at position 132836061 on chromosome 6 referenced to B73AGPv04, preferably detectable by marker sequence of SEQ ID NO: 318,
a “G” at position 132836056 on chromosome 6 referenced to B73AGPv04, preferably detectable by marker sequence of SEQ ID NO: 319,
a “C” at position 132836050 on chromosome 6 referenced to B73AGPv04, preferably detectable by marker sequence of SEQ ID NO: 320,
a “CGT” at position 132836047 on chromosome 6 referenced to B73AGPv04, preferably detectable by marker sequence of SEQ ID NO: 321 and/or 322,
a “G” at position 132836034 on chromosome 6 referenced to B73AGPv04, preferably detectable by marker sequence of SEQ ID NO: 323,
a “G” at position 132836031 on chromosome 6 referenced to B73AGPv04, preferably detectable by marker sequence of SEQ ID NO: 324,
a “T” at position 132836019 on chromosome 6 referenced to B73AGPv04, preferably detectable by marker sequence of SEQ ID NO: 325,
a “G” at position 132836008 on chromosome 6 referenced to B73AGPv04, preferably detectable by marker sequence of SEQ ID NO: 326,
a “G” at position 132835978 on chromosome 6 referenced to B73AGPv04, preferably detectable by marker sequence of SEQ ID NO: 327,
a “G” at position 132835910 on chromosome 6 referenced to B73AGPv04, preferably detectable by marker sequence of SEQ ID NO: 328,
a “G” at position 132835851 on chromosome 6 referenced to B73AGPv04, preferably detectable by marker sequence of SEQ ID NO: 329,
a “C” at position 132835819 on chromosome 6 referenced to B73AGPv04, preferably detectable by marker sequence of SEQ ID NO: 330,
a “G” at position 132835803 on chromosome 6 referenced to B73AGPv04, preferably detectable by marker sequence of SEQ ID NO: 331,
a “T” at position 132835793 on chromosome 6 referenced to B73AGPv04, preferably detectable by marker sequence of SEQ ID NO: 332,
a “C” at position 132835788 on chromosome 6 referenced to B73AGPv04, preferably detectable by marker sequence of SEQ ID NO: 333,
an “A” at position 132835781 on chromosome 6 referenced to B73AGPv04, preferably detectable by marker sequence of SEQ ID NO: 334,
an “A” at position 132835779 on chromosome 6 referenced to B73AGPv04, preferably detectable by marker sequence of SEQ ID NO: 335,
a “T” at position 132835776 on chromosome 6 referenced to B73AGPv04, preferably detectable by marker sequence of SEQ ID NO: 336,
a “G” at position 132835746 on chromosome 6 referenced to B73AGPv04, preferably detectable by marker sequence of SEQ ID NO: 337,
an “A” at position 132835729 on chromosome 6 referenced to B73AGPv04, preferably detectable by marker sequence of SEQ ID NO: 338,
a “C” at position 132835728 on chromosome 6 referenced to B73AGPv04, preferably detectable by marker sequence of SEQ ID NO: 339, an insertion of “GACATC” at position 132835722 on chromosome 6 referenced to B73AGPv04, preferably detectable by marker sequence of SEQ ID NO: 340 and/or 341,
a “T” at position 132835713 on chromosome 6 referenced to B73AGPv04, preferably detectable by marker sequence of SEQ ID NO: 342,
a deletion of “GAA” at position 132835708 on chromosome 6 referenced to B73AGPv04, preferably detectable by marker sequence of SEQ ID NO: 343 and/or 344,
a deletion of “ATA” at position 132835705 on chromosome 6 referenced to B73AGPv04, preferably detectable by marker sequence of SEQ ID NO: 343 and/or 344,
a deletion of “GAT” at position 132835702 on chromosome 6 referenced to B73AGPv04, preferably detectable by marker sequence of SEQ ID NO: 343 and/or 344,
a “G” at position 132835693 on chromosome 6 referenced to B73AGPv04, preferably detectable by marker sequence of SEQ ID NO: 345,
a “T” at position 132835681 on chromosome 6 referenced to B73AGPv04, preferably detectable by marker sequence of SEQ ID NO: 346,
a “C” at position 132835671 on chromosome 6 referenced to B73AGPv04, preferably detectable by marker sequence of SEQ ID NO: 347,
a “T” at position 132835666 on chromosome 6 referenced to B73AGPv04, preferably detectable by marker sequence of SEQ ID NO: 348,
an “A” at position 132835271 on chromosome 6 referenced to B73AGPv04, preferably detectable by marker sequence of SEQ ID NO: 349,
a “GG” at position 132835267 on chromosome 6 referenced to B73AGPv04, preferably detectable by marker sequence of SEQ ID NO: 350 and/or 351,
a “T” at position 132835147 on chromosome 6 referenced to B73AGPv04, preferably detectable by marker sequence of SEQ ID NO: 352,
a “C” at position 132835031 on chromosome 6 referenced to B73AGPv04, preferably detectable by marker sequence of SEQ ID NO: 353,
an “A” at position 132834960 on chromosome 6 referenced to B73AGPv04, preferably detectable by marker sequence of SEQ ID NO: 354,
an “A” at position 132834951 on chromosome 6 referenced to B73AGPv04, preferably detectable by marker sequence of SEQ ID NO: 355,
a “AC” at position 132834946 on chromosome 6 referenced to B73AGPv04, preferably detectable by marker sequence of SEQ ID NO: 356 and/or 357,
an “A” at position 132834942 on chromosome 6 referenced to B73AGPv04, preferably detectable by marker sequence of SEQ ID NO: 358,
an “A” at position 132834929 on chromosome 6 referenced to B73AGPv04, preferably detectable by marker sequence of SEQ ID NO: 359,
a “T” at position 132834927 on chromosome 6 referenced to B73AGPv04, preferably detectable by marker sequence of SEQ ID NO: 360,
an “A” at position 132834924 on chromosome 6 referenced to B73AGPv04, preferably detectable by marker sequence of SEQ ID NO: 361,
a “C” at position 132834919 on chromosome 6 referenced to B73AGPv04, preferably detectable by marker sequence of SEQ ID NO: 362,
a “T” at position 132834908 on chromosome 6 referenced to B73AGPv04, preferably detectable by marker sequence of SEQ ID NO: 363,
an “A” at position 132834899 on chromosome 6 referenced to B73AGPv04, preferably detectable by marker sequence of SEQ ID NO: 364,
a “C” at position 132834754 on chromosome 6 referenced to B73AGPv04, preferably detectable by marker sequence of SEQ ID NO: 365,
a “C” at position 132834753 on chromosome 6 referenced to B73AGPv04, preferably detectable by marker sequence of SEQ ID NO: 366,
a “C” at position 132834748 on chromosome 6 referenced to B73AGPv04, preferably detectable by marker sequence of SEQ ID NO: 367,
an “A” at position 132834721 on chromosome 6 referenced to B73AGPv04, preferably detectable by marker sequence of SEQ ID NO: 368,
an “A” at position 132834632 on chromosome 6 referenced to B73AGPv04, preferably detectable by marker sequence of SEQ ID NO: 369,
a “C” at position 132834622 on chromosome 6 referenced to B73AGPv04, preferably detectable by marker sequence of SEQ ID NO: 370,
a “G” at position 132834615 on chromosome 6 referenced to B73AGPv04, preferably detectable by marker sequence of SEQ ID NO: 371,
a “C” at position 132834602 on chromosome 6 referenced to B73AGPv04,
a “G” at position 132834589 on chromosome 6 referenced to B73AGPv04, preferably detectable by marker sequence of SEQ ID NO: 372,
a “T” at position 132834581 on chromosome 6 referenced to B73AGPv04, preferably detectable by marker sequence of SEQ ID NO: 373,
an “A” at position 132834577 on chromosome 6 referenced to B73AGPv04, preferably detectable by marker sequence of SEQ ID NO: 374,
a “G” at position 132834569 on chromosome 6 referenced to B73AGPv04, preferably detectable by marker sequence of SEQ ID NO: 375,
a “C” at position 132834539 on chromosome 6 referenced to B73AGPv04, preferably detectable by marker sequence of SEQ ID NO: 376,
a “GAG” at position 132834532 on chromosome 6 referenced to B73AGPv04, preferably detectable by marker sequence of SEQ ID NO: 377 and/or 378,
a “T” at position 132834494 on chromosome 6 referenced to B73AGPv04, preferably detectable by marker sequence of SEQ ID NO: 379,
a “T” at position 132834431 on chromosome 6 referenced to B73AGPv04, preferably detectable by marker sequence of SEQ ID NO: 380,
a “G” at position 132834389 on chromosome 6 referenced to B73AGPv04, preferably detectable by marker sequence of SEQ ID NO: 381,
a “T” at position 132834208 on chromosome 6 referenced to B73AGPv04, preferably detectable by marker sequence of SEQ ID NO: 382,
a “T” at position 132831963 on chromosome 6 referenced to B73AGPv04, preferably detectable by marker sequence of SEQ ID NO: 383,
a “G” at position 132831924 on chromosome 6 referenced to B73AGPv04, preferably detectable by marker sequence of SEQ ID NO: 384,
an “A” at position 132831915 on chromosome 6 referenced to B73AGPv04, preferably detectable by marker sequence of SEQ ID NO: 385,
a “G” at position 132831839 on chromosome 6 referenced to B73AGPv04, preferably detectable by marker sequence of SEQ ID NO: 386,
a “T” at position 132829096 on chromosome 6 referenced to B73AGPv04, preferably detectable by marker sequence of SEQ ID NO: 387,
an “A” at position 132828997 on chromosome 6 referenced to B73AGPv04, preferably detectable by marker sequence of SEQ ID NO: 388,
a “G” at position 132828958 on chromosome 6 referenced to B73AGPv04, preferably detectable by marker sequence of SEQ ID NO: 389,
a “G” at position 132828950 on chromosome 6 referenced to B73AGPv04, preferably detectable by marker sequence of SEQ ID NO: 390,
a “C” at position 132828867 on chromosome 6 referenced to B73AGPv04, preferably detectable by marker sequence of SEQ ID NO: 391,
an “A” at position 132827606 on chromosome 6 referenced to B73AGPv04, preferably detectable by marker sequence of SEQ ID NO: 392,
an insertion of “AGTTCATAATAAAGTGATAGAGTT” (SEQ ID NO: 414) at position 132827596 on chromosome 6 referenced to B73AGPv04, preferably detectable by marker sequence of SEQ ID NO: 393 and/or 394,
a “T” at position 132827573 on chromosome 6 referenced to B73AGPv04, preferably detectable by marker sequence of SEQ ID NO: 395,
a “G” at position 132827566 on chromosome 6 referenced to B73AGPv04, preferably detectable by marker sequence of SEQ ID NO: 396,
an “A” at position 132827549 on chromosome 6 referenced to B73AGPv04, preferably detectable by marker sequence of SEQ ID NO: 397,
a “TAT” at position 132827545 on chromosome 6 referenced to B73AGPv04, preferably detectable by marker sequence of SEQ ID NO: 398 and/or 399,
a “C” at position 132827302 on chromosome 6 referenced to B73AGPv04, preferably detectable by marker sequence of SEQ ID NO: 400,
a “T” at position 132826983 on chromosome 6 referenced to B73AGPv04, preferably detectable by marker sequence of SEQ ID NO: 401,
a “T” at position 132826940 on chromosome 6 referenced to B73AGPv04, preferably detectable by marker sequence of SEQ ID NO: 402,
a “T” at position 1333040380 on chromosome 6 referenced to B73AGPv04, preferably detectable by marker sequence of SEQ ID NO: 403,
a “C” at position 1333040382 on chromosome 6 referenced to B73AGPv04, preferably detectable by marker sequence of SEQ ID NO: 404, or
a “T” at position 133040760 on chromosome 6 referenced to B73AGPv04, preferably detectable by marker sequence of SEQ ID NO: 405.

In some embodiments, the nucleic acid molecule encoding a Rcg1 resistance allele has a haplotype comprising a single nucleotide polymorphism of “C” at position 413 referenced to SEQ ID NO: 50. In some embodiments, the nucleic acid molecule encoding a Rcg1 resistance allele has a haplotype comprising a single nucleotide polymorphism of “C” at position 958 referenced to SEQ ID NO: 50. In some embodiments, the nucleic acid molecule encoding a Rcg1 resistance allele has a haplotype comprising a single nucleotide polymorphism of “C” at position 971 referenced to SEQ ID NO: 50. In some embodiments, the nucleic acid molecule encoding a Rcg1 resistance allele has a haplotype comprising a single nucleotide polymorphism of “T” at position 1099 referenced to SEQ ID NO: 50. In some embodiments, the nucleic acid molecule encoding a Rcg1 resistance allele has a haplotype comprising a single nucleotide polymorphism of “A” at position 1154 referenced to SEQ ID NO: 50. In some embodiments, the nucleic acid molecule encoding a Rcg1 resistance allele has a haplotype comprising a single nucleotide polymorphism of “T” at position 1250 referenced to SEQ ID NO: 50. In some embodiments, the nucleic acid molecule encoding a Rcg1 resistance allele has a haplotype comprising a single nucleotide polymorphism of “G” at position 1607 referenced to SEQ ID NO: 50. In some embodiments, the nucleic acid molecule encoding a Rcg1 resistance allele has a haplotype comprising a single nucleotide polymorphism of “G” at position 2001 referenced to SEQ ID NO: 50. In some embodiments, the nucleic acid molecule encoding a Rcg1 resistance allele has a haplotype comprising a single nucleotide polymorphism of “A” at position 2598 referenced to SEQ ID NO: 50. In some embodiments, the nucleic acid molecule encoding a Rcg1 resistance allele has a haplotype comprising a single nucleotide polymorphism of “A” at position 3342 referenced to SEQ ID NO: 50.

In a related aspect is provided a maize plant comprising a resistance locus on chromosome 6 associated with anthracnose stalk rot resistance, but does not further comprise a resistance locus on chromosome 4, or a nucleic acid comprising sequence from the resistance locus on chromosome 4. The maize plant comprises one or more of the following nucleic acids:
i. a nucleotide sequence of SEQ ID NO: 209, 212, 215, 218, 221, 224, 227, 230, 233, 236, 239, 242, 245, 248, 251, 254, 257, 260, 263, 266 or 269, preferably of SEQ ID NO: 266 or 269, or
ii. a nucleotide sequence having a coding sequence of SEQ ID NO: 210, 213, 216, 219, 222, 225, 228, 231, 234, 237, 240, 243, 246, 249, 252, 255, 258, 261, 264, 267 or 270, preferably of SEQ ID NO: 267 or 270,
iii. a nucleotide sequence which is complementary to a sequence from i. or ii.,
iv. a nucleotide sequence which has at least 80% or 85%, preferably at least 90%, 91%, 92%, 93%, 94% or 95%, more preferably at least 96%, 97%, 98% or 99% identity with a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 211, 214, 217, 220, 223, 226, 229, 232, 235, 238, 241, 244, 247, 250, 253, 256, 259, 262, 265, 268 or 271, preferably 268 or 271,
viii. a nucleotide sequence which encodes for a protein which has at least 80% or 8500, preferably at least 90%, 91%, 92%, 93%, 94% or 95%, more preferably at least 96%, 97%, 98% or 99% identity with a sequence of SEQ ID NOs: 211, 214, 217, 220, 223, 226, 229, 232, 235, 238, 241, 244, 247, 250, 253, 256, 259, 262, 265, 268 or 271, preferably 268 or 271.

Alternatively, the maize plant comprises one or more of the following nucleic acids:
i. a nucleotide sequence of SEQ ID NO: 1, 20, 23, 44 or 47,
ii. a nucleotide sequence which is complementary to a sequence from (i),
iii. a nucleotide sequence which has at least 80% or 85%, preferably at least 90%, 91%, 92%, 93%, 94% or 95%, more preferably at least 96%, 97%, 98% or 99% identity with a sequence from (i) or (ii),
iv. a nucleotide sequence which differs from a nucleic acid sequence according to (i), (ii) or (iii) depending on the degeneracy of the genetic code,
v. a nucleotide sequence which hybridizes with a nucleic acid sequence according to i., ii. or iii. under stringent conditions,
vi. a nucleotide sequence which encodes for a protein comprising the sequence of SEQ ID NO: 11-19, 22, 34-43, 46 or 49,
vii. a nucleotide sequence which encodes for a protein which has at least 80% or 85%, preferably at least 90%, 91%, 92%, 93%, 94% or 95%, more preferably at least 96%, 97%, 98% or 99% identity with a sequence of SEQ ID NOs: 11-19, 22, 34-43, 46 or 49.

In various embodiments, the nucleic acid molecule does not produce the amplicon according to SEQ ID NO: 119 upon polymerase chain reaction amplification by means of primers of SEQ ID NO: 120 and 121, primers of SEQ ID NO: 122 and 123, or primers of SEQ ID NO: 124 and 125.

In various embodiments, the nucleic acid molecule does not produce an amplicon selected from the group consisting of SEQ ID NOs: 94, 101, 106, 109, and 114 upon polymerase chain reaction amplification by means of primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118.

In another aspect is provided a maize plant comprising two resistance loci associated with anthracnose stalk rot resistance, wherein one resistance locus is located on chromosome 6, and wherein the other the resistance locus is located on chromosome 4. The maize plant comprises one or more of the following nucleic acids:
i. a nucleotide sequence of SEQ ID NO: 209, 212, 215, 218, 221, 224, 227, 230, 233, 236, 239, 242, 245, 248, 251, 254, 257, 260, 263, 266 or 269, preferably of SEQ ID NO: 266 or 269,
ii. a nucleotide sequence having a coding sequence of SEQ ID NO: 210, 213, 216, 219, 222, 225, 228, 231, 234, 237, 240, 243, 246, 249, 252, 255, 258, 261, 264, 267 or 270, preferably of SEQ ID NO: 267 or 270,
iii. a nucleotide sequence which is complementary to a sequence from i. or ii.,
iv. a nucleotide sequence which has at least 80% or 85%, preferably at least 90%, 91%, 92%, 93%, 94% or 95%, more preferably at least 96%, 97%, 98% or 99% identity with a sequence from i. or ii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 211, 214, 217, 220, 223, 226, 229, 232, 235, 238, 241, 244, 247, 250, 253, 256, 259, 262, 265, 268 or 271, preferably SEQ ID NO: 268 or 271,
viii. a nucleotide sequence which encodes for a protein which has at least 80% or 85%, preferably at least 90%, 91%, 92%, 93%, 94% or 95%, more preferably at least 96%, 97%, 98% or 99% identity with a sequence of SEQ ID NOs: 211, 214, 217, 220, 223, 226, 229, 232, 235, 238, 241, 244, 247, 250, 253, 256, 259, 262, 265, 268 or 271, preferably SEQ ID NO: 268 or 271.

The maize plant also comprises a nucleic acid molecule encoding an anthracnose stalk rot resistance allele having a haplotype comprising one or more nucleotide polymorphism selected from the group consisting of:
an “A” at position 132836954 on chromosome 6 referenced to B73AGPv04, preferably detectable by marker sequence of SEQ ID NO: 273,
a “T” at position 132836944 on chromosome 6 referenced to B73AGPv04, preferably detectable by marker sequence of SEQ ID NO: 274,
an “A” at position 132836869 on chromosome 6 referenced to B73AGPv04, preferably detectable by marker sequence of SEQ ID NO: 275,
a “T” at position 132836849 on chromosome 6 referenced to B73AGPv04, preferably detectable by marker sequence of SEQ ID NO: 276,
a “T” at position 132836845 on chromosome 6 referenced to B73AGPv04, preferably detectable by marker sequence of SEQ ID NO: 277,
a “G” at position 132836840 on chromosome 6 referenced to B73AGPv04, preferably detectable by marker sequence of SEQ ID NO: 278,
a “T” at position 132836830 on chromosome 6 referenced to B73AGPv04, preferably detectable by marker sequence of SEQ ID NO: 279,
an “A” at position 132836810 on chromosome 6 referenced to B73AGPv04, preferably detectable by marker sequence of SEQ ID NO: 280,
a “CA” at position 132836805 on chromosome 6 referenced to B73AGPv04, preferably detectable by marker sequence of SEQ ID NO: 281 and/or 282,
a deletion of “ATC” at position 132836802 on chromosome 6 referenced to B73AGPv04, preferably detectable by marker sequence of SEQ ID NO: 283 and/or 284,
a deletion of “ATT” at position 132836799 on chromosome 6 referenced to B73AGPv04, preferably detectable by marker sequence of SEQ ID NO: 283 and/or 284,
an “A” at position 132836791 on chromosome 6 referenced to B73AGPv04, preferably detectable by marker sequence of SEQ ID NO: 285,
a “CTG” at position 132836787 on chromosome 6 referenced to B73AGPv04, preferably detectable by marker sequence of SEQ ID NO: 286 and/or 287,
a “GA” at position 132836781 on chromosome 6 referenced to B73AGPv04, preferably detectable by marker sequence of SEQ ID NO: 288 and/or 289,
a “T” at position 132836776 on chromosome 6 referenced to B73AGPv04, preferably detectable by marker sequence of SEQ ID NO: 290,
a “AG” at position 132836767 on chromosome 6 referenced to B73AGPv04, preferably detectable by marker sequence of SEQ ID NO: 291 and/or 292,
an “A” at position 132836762 on chromosome 6 referenced to B73AGPv04, preferably detectable by marker sequence of SEQ ID NO: 293,
an insertion of “CGCCAA” and an “A” at position 132836679 on chromosome 6 referenced to B73AGPv04, preferably detectable by marker sequence of SEQ ID NO: 294 and/or 295,
an “A” at position 132836678 on chromosome 6 referenced to B73AGPv04, preferably detectable by marker sequence of SEQ ID NO: 296,
a “G” at position 132836670 on chromosome 6 referenced to B73AGPv04, preferably detectable by marker sequence of SEQ ID NO: 297,
a “T” at position 132836667 on chromosome 6 referenced to B73AGPv04, preferably detectable by marker sequence of SEQ ID NO: 298,
an “A” at position 132836665 on chromosome 6 referenced to B73AGPv04, preferably detectable by marker sequence of SEQ ID NO: 299,
a “C” at position 132836662 on chromosome 6 referenced to B73AGPv04, preferably detectable by marker sequence of SEQ ID NO: 300,
a “T” at position 132836660 on chromosome 6 referenced to B73AGPv04, preferably detectable by marker sequence of SEQ ID NO: 301,
a “G” at position 132836651 on chromosome 6 referenced to B73AGPv04, preferably detectable by marker sequence of SEQ ID NO: 302,
a deletion of “AAT” at position 132836643 on chromosome 6 referenced to B73AGPv04, preferably detectable by marker sequence of SEQ ID NO: 303 and/or 304,
a deletion of “GCCATG” at position 132836636 on chromosome 6 referenced to B73AGPv04, preferably detectable by marker sequence of SEQ ID NO: 305 and/or 306,
a “AG” at position 132836622 on chromosome 6 referenced to B73AGPv04, preferably detectable by marker sequence of SEQ ID NO: 307 and/or 308,
a “AC” at position 132836620 on chromosome 6 referenced to B73AGPv04, preferably detectable by marker sequence of SEQ ID NO: 309 and/or 310,
a “G” at position 132836612 on chromosome 6 referenced to B73AGPv04, preferably detectable by marker sequence of SEQ ID NO: 311,
a “G” at position 132836603 on chromosome 6 referenced to B73AGPv04, preferably detectable by marker sequence of SEQ ID NO: 312,
a “C” at position 132836591 on chromosome 6 referenced to B73AGPv04, preferably detectable by marker sequence of SEQ ID NO: 313,
an “A” at position 132836586 on chromosome 6 referenced to B73AGPv04, preferably detectable by marker sequence of SEQ ID NO: 314,
a “G” at position 132836580 on chromosome 6 referenced to B73AGPv04, preferably detectable by marker sequence of SEQ ID NO: 315,
an “A” at position 132836069 on chromosome 6 referenced to B73AGPv04, preferably detectable by marker sequence of SEQ ID NO: 316,
an “A” at position 132836063 on chromosome 6 referenced to B73AGPv04, preferably detectable by marker sequence of SEQ ID NO: 317,
a “G” at position 132836061 on chromosome 6 referenced to B73AGPv04, preferably detectable by marker sequence of SEQ ID NO: 318,
a “G” at position 132836056 on chromosome 6 referenced to B73AGPv04, preferably detectable by marker sequence of SEQ ID NO: 319,
a “C” at position 132836050 on chromosome 6 referenced to B73AGPv04, preferably detectable by marker sequence of SEQ ID NO: 320,
a “CGT” at position 132836047 on chromosome 6 referenced to B73AGPv04, preferably detectable by marker sequence of SEQ ID NO: 321 and/or 322,
a “G” at position 132836034 on chromosome 6 referenced to B73AGPv04, preferably detectable by marker sequence of SEQ ID NO: 323,
a “G” at position 132836031 on chromosome 6 referenced to B73AGPv04, preferably detectable by marker sequence of SEQ ID NO: 324,
a “T” at position 132836019 on chromosome 6 referenced to B73AGPv04, preferably detectable by marker sequence of SEQ ID NO: 325,
a “G” at position 132836008 on chromosome 6 referenced to B73AGPv04, preferably detectable by marker sequence of SEQ ID NO: 326,
a “G” at position 132835978 on chromosome 6 referenced to B73AGPv04, preferably detectable by marker sequence of SEQ ID NO: 327,
a “G” at position 132835910 on chromosome 6 referenced to B73AGPv04, preferably detectable by marker sequence of SEQ ID NO: 328,
a “G” at position 132835851 on chromosome 6 referenced to B73AGPv04, preferably detectable by marker sequence of SEQ ID NO: 329,
a “C” at position 132835819 on chromosome 6 referenced to B73AGPv04, preferably detectable by marker sequence of SEQ ID NO: 330,
a “G” at position 132835803 on chromosome 6 referenced to B73AGPv04, preferably detectable by marker sequence of SEQ ID NO: 331,
a “T” at position 132835793 on chromosome 6 referenced to B73AGPv04, preferably detectable by marker sequence of SEQ ID NO: 332,
a “C” at position 132835788 on chromosome 6 referenced to B73AGPv04, preferably detectable by marker sequence of SEQ ID NO: 333,
an “A” at position 132835781 on chromosome 6 referenced to B73AGPv04, preferably detectable by marker sequence of SEQ ID NO: 334,
an “A” at position 132835779 on chromosome 6 referenced to B73AGPv04, preferably detectable by marker sequence of SEQ ID NO: 335,
a “T” at position 132835776 on chromosome 6 referenced to B73AGPv04, preferably detectable by marker sequence of SEQ ID NO: 336,
a “G” at position 132835746 on chromosome 6 referenced to B73AGPv04, preferably detectable by marker sequence of SEQ ID NO: 337,
an “A” at position 132835729 on chromosome 6 referenced to B73AGPv04, preferably detectable by marker sequence of SEQ ID NO: 338,
a “C” at position 132835728 on chromosome 6 referenced to B73AGPv04, preferably detectable by marker sequence of SEQ ID NO: 339,
an insertion of “GACATC” at position 132835722 on chromosome 6 referenced to B73AGPv04, preferably detectable by marker sequence of SEQ ID NO: 340 and/or 341,
a “T” at position 132835713 on chromosome 6 referenced to B73AGPv04, preferably detectable by marker sequence of SEQ ID NO: 342,
a deletion of “GAA” at position 132835708 on chromosome 6 referenced to B73AGPv04, preferably detectable by marker sequence of SEQ ID NO: 343 and/or 344,
a deletion of “ATA” at position 132835705 on chromosome 6 referenced to B73AGPv04, preferably detectable by marker sequence of SEQ ID NO: 343 and/or 344,
a deletion of “GAT” at position 132835702 on chromosome 6 referenced to B73AGPv04, preferably detectable by marker sequence of SEQ ID NO: 343 and/or 344,
a “G” at position 132835693 on chromosome 6 referenced to B73AGPv04, preferably detectable by marker sequence of SEQ ID NO: 345,
a “T” at position 132835681 on chromosome 6 referenced to B73AGPv04, preferably detectable by marker sequence of SEQ ID NO: 346,
a “C” at position 132835671 on chromosome 6 referenced to B73AGPv04, preferably detectable by marker sequence of SEQ ID NO: 347,
a “T” at position 132835666 on chromosome 6 referenced to B73AGPv04, preferably detectable by marker sequence of SEQ ID NO: 348,
an “A” at position 132835271 on chromosome 6 referenced to B73AGPv04, preferably detectable by marker sequence of SEQ ID NO: 349,
a “GG” at position 132835267 on chromosome 6 referenced to B73AGPv04, preferably detectable by marker sequence of SEQ ID NO: 350 and/or 351,
a “T” at position 132835147 on chromosome 6 referenced to B73AGPv04, preferably detectable by marker sequence of SEQ ID NO: 352,
a “C” at position 132835031 on chromosome 6 referenced to B73AGPv04, preferably detectable by marker sequence of SEQ ID NO: 353,
an “A” at position 132834960 on chromosome 6 referenced to B73AGPv04, preferably detectable by marker sequence of SEQ ID NO: 354,
an “A” at position 132834951 on chromosome 6 referenced to B73AGPv04, preferably detectable by marker sequence of SEQ ID NO: 355,
a “AC” at position 132834946 on chromosome 6 referenced to B73AGPv04, preferably detectable by marker sequence of SEQ ID NO: 356 and/or 357,
an “A” at position 132834942 on chromosome 6 referenced to B73AGPv04, preferably detectable by marker sequence of SEQ ID NO: 358,
an “A” at position 132834929 on chromosome 6 referenced to B73AGPv04, preferably detectable by marker sequence of SEQ ID NO: 359,
a “T” at position 132834927 on chromosome 6 referenced to B73AGPv04, preferably detectable by marker sequence of SEQ ID NO: 360,
an “A” at position 132834924 on chromosome 6 referenced to B73AGPv04, preferably detectable by marker sequence of SEQ ID NO: 361,
a “C” at position 132834919 on chromosome 6 referenced to B73AGPv04, preferably detectable by marker sequence of SEQ ID NO: 362,
a “T” at position 132834908 on chromosome 6 referenced to B73AGPv04, preferably detectable by marker sequence of SEQ ID NO: 363,
an “A” at position 132834899 on chromosome 6 referenced to B73AGPv04, preferably detectable by marker sequence of SEQ ID NO: 364,
a “C” at position 132834754 on chromosome 6 referenced to B73AGPv04, preferably detectable by marker sequence of SEQ ID NO: 365,
a “C” at position 132834753 on chromosome 6 referenced to B73AGPv04, preferably detectable by marker sequence of SEQ ID NO: 366,
a “C” at position 132834748 on chromosome 6 referenced to B73AGPv04, preferably detectable by marker sequence of SEQ ID NO: 367,
an “A” at position 132834721 on chromosome 6 referenced to B73AGPv04, preferably detectable by marker sequence of SEQ ID NO: 368,
an “A” at position 132834632 on chromosome 6 referenced to B73AGPv04, preferably detectable by marker sequence of SEQ ID NO: 369,
a “C” at position 132834622 on chromosome 6 referenced to B73AGPv04, preferably detectable by marker sequence of SEQ ID NO: 370,
a “G” at position 132834615 on chromosome 6 referenced to B73AGPv04, preferably detectable by marker sequence of SEQ ID NO: 371,
a “C” at position 132834602 on chromosome 6 referenced to B73AGPv04,
a “G” at position 132834589 on chromosome 6 referenced to B73AGPv04, preferably detectable by marker sequence of SEQ ID NO: 372,
a “T” at position 132834581 on chromosome 6 referenced to B73AGPv04, preferably detectable by marker sequence of SEQ ID NO: 373,
an “A” at position 132834577 on chromosome 6 referenced to B73AGPv04, preferably detectable by marker sequence of SEQ ID NO: 374,
a “G” at position 132834569 on chromosome 6 referenced to B73AGPv04, preferably detectable by marker sequence of SEQ ID NO: 375,
a “C” at position 132834539 on chromosome 6 referenced to B73AGPv04, preferably detectable by marker sequence of SEQ ID NO: 376,
a “GAG” at position 132834532 on chromosome 6 referenced to B73AGPv04, preferably detectable by marker sequence of SEQ ID NO: 377 and/or 378,
a “T” at position 132834494 on chromosome 6 referenced to B73AGPv04, preferably detectable by marker sequence of SEQ ID NO: 379,
a “T” at position 132834431 on chromosome 6 referenced to B73AGPv04, preferably detectable by marker sequence of SEQ ID NO: 380,
a “G” at position 132834389 on chromosome 6 referenced to B73AGPv04, preferably detectable by marker sequence of SEQ ID NO: 381,
a “T” at position 132834208 on chromosome 6 referenced to B73AGPv04, preferably detectable by marker sequence of SEQ ID NO: 382,
a “T” at position 132831963 on chromosome 6 referenced to B73AGPv04, preferably detectable by marker sequence of SEQ ID NO: 383,
a “G” at position 132831924 on chromosome 6 referenced to B73AGPv04, preferably detectable by marker sequence of SEQ ID NO: 384,
an “A” at position 132831915 on chromosome 6 referenced to B73AGPv04, preferably detectable by marker sequence of SEQ ID NO: 385,
a “G” at position 132831839 on chromosome 6 referenced to B73AGPv04, preferably detectable by marker sequence of SEQ ID NO: 386,
a “T” at position 132829096 on chromosome 6 referenced to B73AGPv04, preferably detectable by marker sequence of SEQ ID NO: 387,
an “A” at position 132828997 on chromosome 6 referenced to B73AGPv04, preferably detectable by marker sequence of SEQ ID NO: 388,
a “G” at position 132828958 on chromosome 6 referenced to B73AGPv04, preferably detectable by marker sequence of SEQ ID NO: 389,
a “G” at position 132828950 on chromosome 6 referenced to B73AGPv04, preferably detectable by marker sequence of SEQ ID NO: 390,
a “C” at position 132828867 on chromosome 6 referenced to B73AGPv04, preferably detectable by marker sequence of SEQ ID NO: 391,
an “A” at position 132827606 on chromosome 6 referenced to B73AGPv04, preferably detectable by marker sequence of SEQ ID NO: 392,
an insertion of “AGTTCATAATAAAGTGATAGAGTT” (SEQ ID NO: 414) at position 132827596 on chromosome 6 referenced to B73AGPv04, preferably detectable by marker sequence of SEQ ID NO: 393 and/or 394,
a “T” at position 132827573 on chromosome 6 referenced to B73AGPv04, preferably detectable by marker sequence of SEQ ID NO: 395,
a “G” at position 132827566 on chromosome 6 referenced to B73AGPv04, preferably detectable by marker sequence of SEQ ID NO: 396,
an “A” at position 132827549 on chromosome 6 referenced to B73AGPv04, preferably detectable by marker sequence of SEQ ID NO: 397,
a “TAT” at position 132827545 on chromosome 6 referenced to B73AGPv04, preferably detectable by marker sequence of SEQ ID NO: 398 and/or 399,
a “C” at position 132827302 on chromosome 6 referenced to B73AGPv04, preferably detectable by marker sequence of SEQ ID NO: 400,
a “T” at position 132826983 on chromosome 6 referenced to B73AGPv04, preferably detectable by marker sequence of SEQ ID NO: 401,
a “T” at position 132826940 on chromosome 6 referenced to B73AGPv04, preferably detectable by marker sequence of SEQ ID NO: 402,
a “T” at position 1333040380 on chromosome 6 referenced to B73AGPv04, preferably detectable by marker sequence of SEQ ID NO: 403,
a “C” at position 1333040382 on chromosome 6 referenced to B73AGPv04, preferably detectable by marker sequence of SEQ ID NO: 404, or
a “T” at position 133040760 on chromosome 6 referenced to B73AGPv04, preferably detectable by marker sequence of SEQ ID NO: 405.

Alternatively the maize plant comprises one or more of the following nucleic acids:
i. a nucleotide sequence of SEQ ID NO: 1, 20, 23, 44 or 47,
ii. a nucleotide sequence which is complementary to a sequence from (i),
iii. a nucleotide sequence which has at least 80% or 85%, preferably at least 90%, 91%, 92%, 93%, 94% or 95%, more preferably at least 96%, 97%, 98% or 99% identity with a sequence from (i) or (ii),
iv. a nucleotide sequence which differs from a nucleic acid sequence according to (i), (ii) or (iii) depending on the degeneracy of the genetic code,
v. a nucleotide sequence which hybridizes with a nucleic acid sequence according to i., ii. or iii. under stringent conditions,
vi. a nucleotide sequence which encodes for a protein comprising the sequence of SEQ ID NO: 11-19, 22, 34-43, 46 or 49,
vii. a nucleotide sequence which encodes for a protein which has at least 80% or 8500, preferably at least 90%, 91%, 92%, 93%, 94% or 95%, more preferably at least 96%, 97%, 98% or 99% identity with a sequence of SEQ ID NOs: 11-19, 22, 34-43, 46 or 49.

The maize plant also comprises a nucleic acid molecule encoding a Rcg1 resistance allele having a haplotype comprising one or more nucleotide polymorphism selected from the group consisting of:
i. a “C” at position 413 referenced to SEQ ID NO: 50,
ii. a “C” at position 958 referenced to SEQ ID NO: 50,
iii. a “C” at position 971 referenced to SEQ ID NO: 50,
iv. a “T” at position 1099 referenced to SEQ ID NO: 50,
v. an “A” at position 1154 referenced to SEQ ID NO: 50,
vi. a “T” at position 1250 referenced to SEQ ID NO: 50,
vii. a “G” at position 1607 referenced to SEQ ID NO: 50,
viii. a “G” at position 2001 referenced to SEQ ID NO: 50,
ix. an “A” at position 2598 referenced to SEQ ID NO: 50, and
x. an “A” at position 3342 referenced to SEQ ID NO: 50.

Also provided are seeds or plant parts of any of the above maize plants.

### Introgression

When a gene is introgressed by marker-assisted selection, it is not only the gene that is introduced but also the flanking regions (Gepts, Crop Sci, 2002, 42: 1780-1790). This is referred to as “linkage drag.” In the case where the donor plant is highly unrelated to the recipient plant, as in the case of the Rcg1 locus being introgressed from MP305, an exotic source, into elite inbreds, these flanking regions carry additional genes that may code for agronomically undesirable traits. This “linkage drag” may also result in reduced yield or other negative agronomic characteristics even after multiple cycles of backcrossing into the elite corn line. This is also sometimes referred to as “yield drag.” The size of the flanking region can be decreased by additional backcrossing, although this is not always successful, as breeders do not have control over the size of the region or the recombination breakpoints (Young et al., Genetics, 1998, 120:579-585). In classical breeding it is usually only by chance that recombinations are selected that contribute to a reduction in the size of the donor segment (Tanksley et al., Biotechnology, 1989, 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will allow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers. When the exact location of a gene is known, a series of flanking markers surrounding the gene can be utilized to select for recombinations in different population sizes. For example, in smaller population sizes recombinations may be expected further away from the gene, so more distal flanking markers would be required to detect the recombination.

In one aspect is provided a method of introgressing a locus associated with anthracnose stalk rot resistance into a maize plant the method comprising:
a. screening a population with at least one marker to determine if one or more maize plants from the population comprises the locus associated with anthracnose stalk rot resistance, wherein the screening comprises a nucleic acid assay for the detection of at least one marker at a resistance locus on chromosome 6, wherein the resistance locus comprises an anthracnose stalk rot resistance allele having a haplotype comprising one or more nucleotide polymorphism selected from the group consisting of variant nucleotides of Table 13, and/or a nucleic acid assay for the detection of at least one marker at a resistance locus on chromosome 4, wherein the resistance locus comprises a Rcg1 resistance allele having a haplotype comprising one or more nucleotide polymorphism selected from the group consisting of:
a “C” at position 413 referenced to SEQ ID NO: 50,
a “C” at position 958 referenced to SEQ ID NO: 50,
a “C” at position 971 referenced to SEQ ID NO: 50,
a “T” at position 1099 referenced to SEQ ID NO: 50,
an “A” at position 1154 referenced to SEQ ID NO: 50,
a “T” at position 1250 referenced to SEQ ID NO: 50,
a “G” at position 1607 referenced to SEQ ID NO: 50,
a “G” at position 2001 referenced to SEQ ID NO: 50,
an “A” at position 2598 referenced to SEQ ID NO: 50, and
an “A” at position 3342 referenced to SEQ ID NO: 50; and
b. selecting from the population at least one maize plant comprising said locus associated with anthracnose stalk rot resistance; and
c. crossing the at least one maize plant to a second maize plant;
d. evaluating progeny plants for the at least one marker associated with anthracnose stalk rot resistance; and
e. selecting progeny plants possessing the allele associated with anthracnose stalk rot resistance.

In some embodiments, the one or more nucleotide polymorphisms are selected from the group consisting of:

an “A” at position 132836954 on chromosome 6, a “T” at position 132836944 on chromosome 6, an “A” at position 132836869 on chromosome 6, a “T” at position 132836849 on chromosome 6, a “T” at position 132836845 on chromosome 6, a “G” at position 132836840 on chromosome 6, a “T” at position 132836830 on chromosome 6, an “A” at position 132836810 on chromosome 6, a “CA” at position 132836805 on chromosome 6, a deletion of “ATC” at position 132836802 on chromosome 6, a deletion of “ATT” at position 132836799 on chromosome 6, an “A” at position 132836791 on chromosome 6, a “CTG” at position 132836787 on chromosome 6, a “GA” at position 132836781 on chromosome 6, a “T” at position 132836776 on chromosome 6, a “AG” at position 132836767 on chromosome 6, an “A” at position 132836762 on chromosome 6, an insertion of “CGCCAA” and an “A” at position 132836679 on chromosome 6, an “A” at position 132836678 on chromosome 6, a “G” at position 132836670 on chromosome 6, a “T” at position 132836667 on chromosome 6, an “A” at position 132836665 on chromosome 6, a “C” at position 132836662 on chromosome 6, a “T” at position 132836660 on chromosome 6, a “G” at position 132836651 on chromosome 6, a deletion of “AAT” at position 132836643 on chromosome 6, a deletion of “GCCATG” at position 132836636 on chromosome 6, a “AG” at position 132836622 on chromosome 6, a “AC” at position 132836620 on chromosome 6, a “G” at position 132836612 on chromosome 6, a “G” at position 132836603 on chromosome 6, a “C” at position 132836591 on chromosome 6, an “A” at position 132836586 on chromosome 6, a “G” at position 132836580 on chromosome 6, an “A” at position 132836069 on chromosome 6, an “A” at position 132836063 on chromosome 6, a “G” at position 132836061 on chromosome 6, a “G” at position 132836056 on chromosome 6, a “C” at position 132836050 on chromosome 6, a “CGT” at position 132836047 on chromosome 6, a “G” at position 132836034 on chromosome 6, a “G” at position 132836031 on chromosome 6, a “T” at position 132836019 on chromosome 6, a “G” at position 132836008 on chromosome 6, a “G” at position 132835978 on chromosome 6, a “G” at position 132835910 on chromosome 6, a “G” at position 132835851 on chromosome 6, a “C” at position 132835819 on chromosome 6, a “G” at position 132835803 on chromosome 6, a “T” at position 132835793 on chromosome 6, a “C” at position 132835788 on chromosome 6, an “A” at position 132835781 on chromosome 6, an “A” at position 132835779 on chromosome 6, a “T” at position 132835776 on chromosome 6, a “G” at position 132835746 on chromosome 6, an “A” at position 132835729 on chromosome 6, a “C” at position 132835728 on chromosome 6, an insertion of “GACATC” at position 132835722 on chromosome 6, a “T” at position 132835713 on chromosome 6, a deletion of “GAA” at position 132835708 on chromosome 6, a deletion of “ATA” at position 132835705 on chromosome 6, a deletion of “GAT” at position 132835702 on chromosome 6, a “G” at position 132835693 on chromosome 6, a “T” at position 132835681 on chromosome 6, a “C” at position 132835671 on chromosome 6, a “T” at position 132835666 on chromosome 6, an “A” at position 132835271 on chromosome 6, a “GG” at position 132835267 on chromosome 6, a “T” at position 132835147 on chromosome 6, a “C” at position 132835031 on chromosome 6, an “A” at position 132834960 on chromosome 6, an “A” at position 132834951 on chromosome 6, a “AC” at position 132834946 on chromosome 6, an “A” at position 132834942 on chromosome 6, an “A” at position 132834929 on chromosome 6, a “T” at position 132834927 on chromosome 6, an “A” at position 132834924 on chromosome 6, a “C” at position 132834919 on chromosome 6, a “T” at position 132834908 on chromosome 6, an “A” at position 132834899 on chromosome 6, a “C” at position 132834754 on chromosome 6, a “C” at position 132834753 on chromosome 6, a “C” at position 132834748 on chromosome 6, an “A” at position 132834721 on chromosome 6, an “A” at position 132834632 on chromosome 6, a “C” at position 132834622 on chromosome 6, a “G” at position 132834615 on chromosome 6, a “C” at position 132834602 on chromosome 6, a “G” at position 132834589 on chromosome 6, a “T” at position 132834581 on chromosome 6, an “A” at position 132834577 on chromosome 6, a “G” at position 132834569 on chromosome 6, a “C” at position 132834539 on chromosome 6, a “GAG” at position 132834532 on chromosome 6, a “T” at position 132834494 on chromosome 6, a “T” at position 132834431 on chromosome 6, a “G” at position 132834389 on chromosome 6, a “T” at position 132834208 on chromosome 6, a “T” at position 132831963 on chromosome 6, a “G” at position 132831924 on chromosome 6, an “A” at position 132831915 on chromosome 6, a “G” at position 132831839 on chromosome 6, a “T” at position 132829096 on chromosome 6, an “A” at position 132828997 on chromosome 6, a “G” at position 132828958 on chromosome 6, a “G” at position 132828950 on chromosome 6, a “C” at position 132828867 on chromosome 6, an “A” at position 132827606 on chromosome 6, an insertion of “AGTTCATAATAAAGTGATAGAGTT” (SEQ ID NO: 414) at position 132827596 on chromosome 6, a “T” at position 132827573 on chromosome 6, a “G” at position 132827566 on chromosome 6, an “A” at position 132827549 on chromosome 6, a “TAT” at position 132827545 on chromosome 6, a “C” at position 132827302 on chromosome 6, a “T” at position 132826983 on chromosome 6, a “T” at position 132826940 on chromosome 6, a “T” at position 1333040380 on chromosome 6, a “C” at position 1333040382 on chromosome 6, or a “T” at position 133040760 on chromosome 6, compared to the position on chromosome 6 of the B73AGPv04 reference genome sequence.

In various embodiments, the at least one marker used for screening is located within 5 cM of any one of the variant nucleotide polymorphisms recited in Table 13 and/or any one of the polymorphisms: “C” at position 413 referenced to SEQ ID NO: 50, a “C” at position 958 referenced to SEQ ID NO: 50, a “C” at position 971 referenced to SEQ ID NO: 50, a “T” at position 1099 referenced to SEQ ID NO: 50, an “A” at position 1154 referenced to SEQ ID NO: 50, a “T” at position 1250 referenced to SEQ ID NO: 50, a “G” at position 1607 referenced to SEQ ID NO: 50, a “G” at position 2001 referenced to SEQ ID NO: 50, an “A” at position 2598 referenced to SEQ ID NO: 50, or an “A” at position 3342 referenced to SEQ ID NO: 50.

In various embodiments, the at least one marker used for screening is located within 1 cM of any one of the variant nucleotide polymorphisms recited in Table 13 and/or any one of the polymorphisms: “C” at position 413 referenced to SEQ ID NO: 50, a “C” at position 958 referenced to SEQ ID NO: 50, a “C” at position 971 referenced to SEQ ID NO: 50, a “T” at position 1099 referenced to SEQ ID NO: 50, an “A” at position 1154 referenced to SEQ ID NO: 50, a “T” at position 1250 referenced to SEQ ID NO: 50, a “G” at position 1607 referenced to SEQ ID NO: 50, a “G” at position 2001 referenced to SEQ ID NO: 50, an “A” at position 2598 referenced to SEQ ID NO: 50, or an “A” at position 3342 referenced to SEQ ID NO: 50.

In various embodiments, the resistance locus on chromosome 4 is located on a chromosomal interval between makers PZE-104102206 and PZE-104132759. In various embodiments, the resistance locus on chromosome 6 is located on a chromosomal interval between makers PZE-106066805 and PZE-106075546. In various embodiments, the resistance locus on chromosome 6 is located on a chromosomal interval between 139646631 and 139889078 numbered according to the B73AGPv05 genome sequence. In various embodiments, the resistance locus of chromosome 6 comprises SEQ ID NO: 272, or a fragment thereof.

In various embodiments, the resistance locus on chromosome 4 does not produce an amplicon selected from the group consisting of SEQ ID NOs: 94, 101, 106, 109, and 114 upon polymerase chain reaction amplification by means of primers of SEQ ID NOs: 95 and 96, primers of SEQ ID NOs: 97 and 98, primers of SEQ ID NOs: 99 and 100, primers of SEQ ID NOs: 101 and 102, primers of SEQ ID NOs: 103 and 104, primers of SEQ ID NOs: 105 and 106, primers of SEQ ID NOs: 107 and 108, primers of SEQ ID NOs: 109 and 110, primers of SEQ ID NOs: 111 and 112, primers of SEQ ID NOs: 113 and 114, primers of SEQ ID NOs: 115 and 116, or primers of SEQ ID NOs: 117 and 118.

In various embodiments, the resistance locus on chromosome 4 is derived from NC262A or NC342. In various embodiments, the resistance locus on chromosome 6 is derived from NC262A.

### Stalk Splitter Apparatus

With respect to the various embodiments of selecting and breeding plants described herein, the plants may be assayed for stalk rot with the use of a stalk splitter apparatus (100). The stalk splitter apparatus can improve the speed and reproducibility of analyzing a corn plant for stalk rot. The stalk splitter apparatus can reproducibly split corn stalks in a manner to allow for improved quantitative analysis of the extent of stalk rot.

The stalk splitter apparatus comprises a body (101), preferably a metallic body, comprising a hole (102) configured to allow a corn stalk (200) to fit into the hole. The metallic body may be comprised of aluminum, steel, copper, or any other suitable metal. The metallic body may be made of a solid metal, such as solid aluminum. The hole may be placed in or near the center of the solid body.

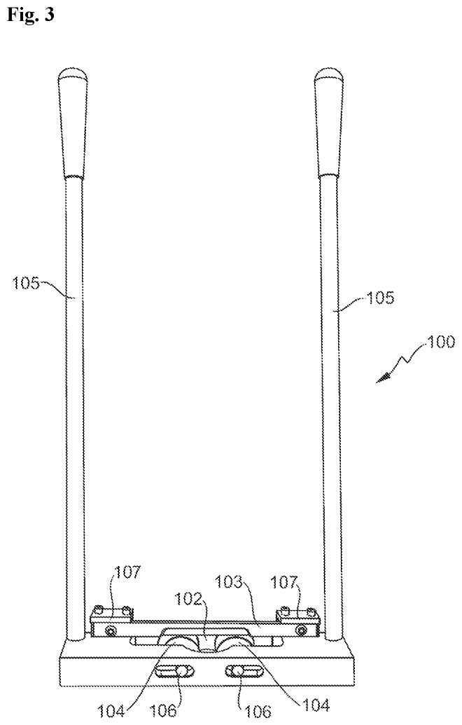

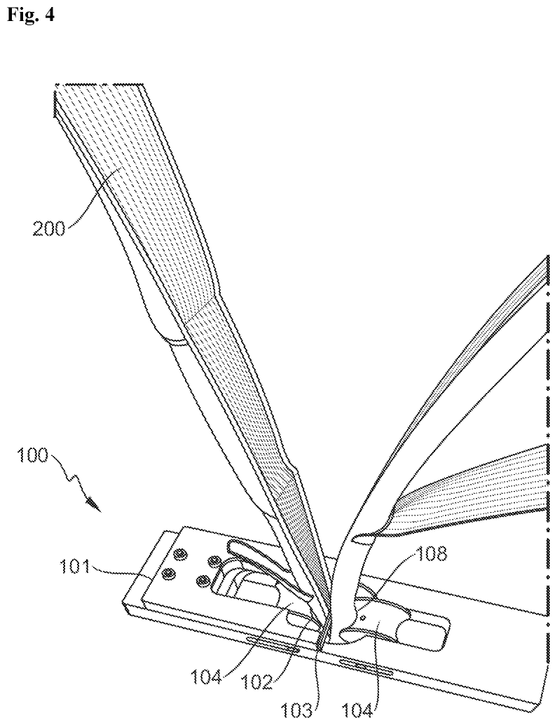

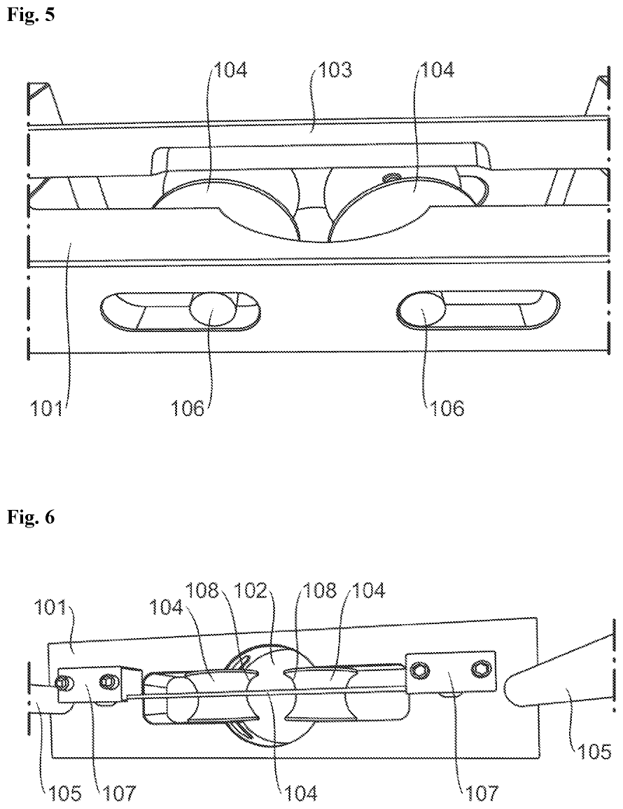

The stalk splitter apparatus comprises a blade (103) positioned above the hole. The blade is of sufficient sharpness and strength so as to be able to split and/or open a corn stalk. In various embodiments, the blade is centered above the hole. In various embodiments, the blade is fixed by mounting elements (107). In various embodiments, the blade is positioned in direction of the shafts of the rollers (106) (as shown in FIG. 3, FIGS. 5 and 6) or perpendicularly to the shafts of the rollers (as shown in FIG. 4), any other direction which allows splitting and/or opening a corn stalk. In various embodiments, the blade is removable for sharpening.

The stalk splitter apparatus comprises two rollers (104) below the blade. The rollers are configured to apply compressive force to the stalk. The compressive force is able to center the stalk into the hole. The stalk splitter apparatus may then be able to cut stalks having variable diameter consistently in the middle. In various embodiments, one or both of the two rollers are tensioned by springs residing in channels drilled horizontally out from the center hole in the body of the splitter on either side of the rollers. In various embodiments, one or both of the two rollers have a concave running surface (108) and/or roughened to center the stalk into the hole and grip the stalk.

To cut a corn stalk, the stalk splitter is initially positioned above the corn stalk such that the top of the corn stalk fits into the hole. Application of downward force allows the stalk splitter to cut the corn stalk. As downward force is applied, the corn stalk moves upward through the hole such that the blade cuts into the center of the stalk.

The stalk splitter apparatus may comprise handles (105) extending from the outer top of the body (101) comprising the hole. The handles may extend up to a length of about 50 cm. The handles may have a length of about 35 cm, 40 cm, 45 cm, 50 cm, 55 cm, or 60 cm. The extended handles may enable a user to push down the stalk all the way through the apparatus to the ground while the user is standing.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1. Identification of Two Significant QTL Relevant to ASR

Hundreds of inbreds were screened for resistance to ASR by artificially infecting stalks with spores of the cultured fungi. The inoculum source was obtained from infected stalks in central Indiana. The inoculum source was purified and cultured on oatmeal agar media. Spores were harvested in 21 days, blended with water, and strained with cheesecloth. Concentrations were normalized to 6×10⁵ ml⁻¹. Plots were 3.3 meters long in rows 0.76 meters wide and replicated twice. Stalks were injected with 0.5 ml of inoculum into the first internode above the brace roots within a week after silking. A Socorex Dosys™ syringe was used whose 3.18 mm diameter needles had their tips filled and a 0.51 mm hole drilled laterally for side flow. Holes 3.97 mm in diameter were pre-drilled in the stalks with a cordless drill. Holes were covered with petroleum jelly after inoculation. Just after black layer maturity was reached, sections of stalks from the ear to the ground were cut off with branch loppers, bundled, barcoded and taken inside to be split with a bandsaw and rated.

The rating was composed of three scores. The first score is the intensity of infection at the inoculation site on a 1 to 9 scale, where 9 represents a completely rotted inoculated internode (lx weight). The second score is the number of nodes darkened more than 10%. The third score is the number of nodes darkened over 50%. A composite score was generated by weighting these scores 1:2:3, respectively. Inbred sources NC262A and NC342 were observed to be resistant for over four years with scores consistently below 10, while susceptible inbreds had scores above 32. Seeds of these inbreds were obtained from the National Genetic Resources Program maize repository at Iowa State (GRIN). They share the same parentage which traces to Coker 811A×C103⁴. These genotypes have not been previously reported to have resistance to ASR. They do not share parentage with known source Mp305. The molecular distinction from known sources is shown in Example 3 below.

A mapping population was generated from the cross NC262A×MN8. (MN8 is a moderately susceptible inbred.) The population was self-pollinated three times to produce a population with 310 F₂:S₂ families. These families were trialed in an augmented incomplete balanced block design for two years with two replications per year. Two inbreds with moderate ASR susceptibility served as the repeated checks in each sub-block. Inoculation and rating were performed as described above. Stalk rot composite scores for the two years as shown in Table 1 below, with higher values signifying more rot in the plant:

TABLE 1
Stalk Rot Composite Scores Over Two Years

| | |
|---|---|
| Mean | 15.77 |
| Min. | 5.15 |
| Max. | 32.21 |
| Var. | 19.19 |

P = 0.028, Passes the Cramer-Von Mises test for normality

A six-plant bulk of the lines was genotyped on an Affymetrix custom maize chip with 30,000 single nucleotide polymorphic loci (SNPs) queried. Composite interval analysis was performed using RQTL (QGene.org). See, Broman K W, Wu H, Sen Ś, Churchill G A (2003) R/qtl: QTL mapping in experimental crosses. Bioinformatics 19:889-890. Composite interval mapping revealed two highly significant QTL, as shown below in Table 2:

TABLE 2
Quantitative trait locus analysis results per R/qtl

| chromo- | Peak | Peak position | Left support | left 1.5 | Right support | Right 1.5 |
|---|---|---|---|---|---|---|
| some | Marker | (v2) | interval | LOD support | int. | LOD support |
| 4 | PZE- | 190,611,189 | 178,597,407 | PZE- | 216,244,367 | PZE- |
| | 104115065 | | | 104102206 | | 104132759 |
| 6 | PZE- | 119,641,655 | 119,158,886 | PZE- | 130,840,928 | PZE- |
| | 106066955 | | | 106066805 | | 106075546 |

| | Support | | | | | |
|---|---|---|---|---|---|---|
| chromo- | int. | Peak | | Est. | Est. | % vari- |
| some | (Mb) | LOD | p-value | (a) | (d) | ation |
| 4 | 37.6 | 7.8 | 1.32E−06 | −1.56 | −0.10 | 7.75 |
| 6 | 11.7 | 12.5 | 2.57E−12 | −2.17 | −0.88 | 16.02 |

Using these results, the progeny of five ASR resistant lines were selected that were heterozygous for the susceptible (MN8) allele on chromosome 4, and/or heterozygous in the support interval on chromosome 6. In this way, the effect of the chromosome 6 locus could be better isolated. These lines were chosen and self-pollinated to find additional recombination in the region and develop a fine map. S4 lines were assayed in the field with three replicates and six plants inoculated per replicate. Five plants per family were also self-pollinated to S5 for later use. 351 lines were tested for ASR resistance in three replicates and five to six plants per replicate, with an augmented incomplete balanced block design used.

Families in the tails of the distribution of stalk rot responses beyond approximately 1.5 of the standard deviation above and below the mean were contrasted for allelic frequency (NC262A SNP vs. MN8's). In those tails (43 lines in the resistant tail and 36 for the susceptible tail), the SNP locus with the highest contrast had a Chi-Squared deviation from the expected 1:1 ratio of 29.3 (P<0.0001) between 85.92 and 86.25 cM on the IBM4 map v2. These boundaries are between SNP markers Affx-90199961 and PZE-106073551, respectively.

The sequence of Affx-90199961 is:

(SEQ ID NO: 415)
ACACATACCACCTTGCTCTCTTCACGAGCTGAAATGCA
CAACCGAACAACGTTTTCCCGCCCATTTTTTTTTTTGT
TCTTCCAGCAGCGTCTTACCAACCNTATAACTGCAGAT
GTGTACAGGCGCGCATACTGACAGAGAGCGTAGAATGN
TACAGCAGTGTGGTCCTTTGTCGATTTTATATTAATTT
GTATGAC.

The sequence of PZE-106073551 is:

(SEQ ID NO: 416)
CTATTACCTCCACCAAAAGGGACGATACATGGCTCTCT
GTCCACCCGTCGTCATGTTTGCCCTATGCTCATGGCTC
ACCTACGTGCTCATCAAGAACTACNACCGCTCTCTTAA
TATCACCGTATTCATAACGGTCGTCTTGGCATTCCTGG
AGTTATACCAGCTCTACTTGTACATAGCCTCTGGTTGG
TTCAAGGTGGC.

The sequence of PZE-104115065 is:

(SEQ ID NO: 417)
TTTTGGTGATCTTCATCTGTTGCTACCAAAAAACAGTT
AAAAGACAAACA[T/C]GCTGCATATCTCAAAGTTTCA
AGGTTGGAGAAGTTCTTTCATGAACTGTC.

NC262A vs. MN8 alleles were also contrasted within families having at least 3 individuals. This was a powerful analysis with 137 such families. All 26 SNPs in the narrowed region were analyzed in a step-wise linear mixed model. SNPs were treated as fixed effects and families treated as random effects in the model:

Score=mean+SNP+family+residual

The result (Table 3) was almost the same as for the Chi-Square analysis involving the most resistant and susceptible tails. A highly significance interval extends from PZE.106072681 (P<1.25E-16) to PZE.106074560 (P<4.26E-14). Within this interval, the largest deviation from the expected 1:1 ratio was between ZmSYNBREED_54946_787 (P<2.10E-25) and ZmSYNBREED_54956_986 (P<1.58E-25).

TABLE 3
Results of Analysis with 137 such Families
in the Highly Significant Interval

| SNP marker name | Chisq | P value |
|---|---|---|
| PZE.106072681 | 68.53 | 1.25E−16 |
| PZE.106073001 | 74.68 | 5.53E−18 |
| PUT.163a.18167596.1294 | 79.51 | 4.81E−19 |
| ZmSYNBREED_54943_173 | 107.52 | 3.42E−25 |
| ZmSYNBREED_54946_787 | 108.48 | 2.10E−25 |
| PZE.106073260 | 108.48 | 2.10E−25 |
| ZmSYNBREED_54949_671 | 108.48 | 2.10E−25 |
| ZmSYNBREED_54956_986 | 109.05 | 1.58E−25 |
| PZE.106073551 | 105.03 | 1.20E−24 |
| ZmSYNBREED_54966_239 | 105.03 | 1.20E−24 |
| PZE.106073623 | 105.03 | 1.20E−24 |
| ZmSYNBREED_54969_202 | 105.03 | 1.20E−24 |
| ZmSYNBREED_54969_513 | 105.03 | 1.20E−24 |
| ZmSYNBREED_54973_806 | 105.69 | 8.60E−25 |
| ZmSYNBREED_54975_531 | 106.56 | 5.55E−25 |
| ZmSYNBREED_54977_728 | 93.08 | 5.01E−22 |
| ZmSYNBREED_54978_371 | 93.08 | 5.01E−22 |
| PZE.106074105 | 92.25 | 7.64E−22 |
| ZmSYNBREED_54980_496 | 92.25 | 7.64E−22 |
| ZmSYNBREED_54981_535 | 90.05 | 2.32E−21 |
| PZE.106074333 | 69.73 | 6.80E−17 |
| ZmSYNBREED_54987_542 | 68.80 | 1.09E−16 |
| PZE.106074560 | 57.05 | 4.26E−14 |

The S5 self-pollinated progeny of 132 families that were heterozygous for all or part of this interval for genotyping were then selected to identify additional recombination within this interval to further delineate the QTL location. 5,022 plants (plus parental checks) were genotyped with 14 gene rich KASP SNPs (LCG, Middlesex, UK) within this region. With the aid of PCR, KASP technology placed different fluors for each of the polymorphisms at the base queried.

Those plants that revealed recombination within the region were then phenotyped. Phenotyping was performed as described apart from the stalk splitting procedure.

Validation and further narrowing of the interval were then carried out using these results.

90 S6 progeny of the above mapping phase, whose genotypes are homozygous in the region and showing past recombination, were selected. Their S5 progenitors had a range of phenotypes in the previous testing phase. These were phenotyped in three replicates of six plants each as follows. All of the plants were carefully inoculated in the lower stalk at flowering with 3×10⁵ Colletotrichum graminicola spores. They were then carefully split as described above at physiological maturity, followed by phenotypic scoring and rating on a 1 to 9 scale as described above. A score of 9 indicates no spreading of fungal growth, while a score of 1 indicates that the plant is completely rotted.

Also, 55 S6 lines that are heterozygous and having been recombined between various markers were chosen. These were phenotyped with 6 replicates of 6 inoculated plants each to account for the variability. The means of these were used for analyses. The means of the 3 allelic classes (AA, AB, and BB) were used for analyses, where A represents a segment from the resistant and B from the susceptible parent.

Orthogonal contrasts within families (near isolines) were inconclusive due to the lower level of replication. A more powerful analysis was undertaken in which all of the more resistant were compared with the more susceptible “tails” of the means. On the same 1 to 9 scale, a priori delimiters of stalk rot rating over 7 or under 5.5 were chosen because visually, these groups represent quite resistant (over 7) versus quite susceptible (under 5.5) sets. For stalk rot, rating at the R2 stage is optimal because rating at a later stage would result in some of the intermediate families presenting as fully susceptible.

The “tails” of S6 lines were contrasted marker by marker using Chi-Square analysis. Under Hardy-Weinberg equilibrium, if there is no marker-trait association, an equal frequency of alleles between the tails at each marker locus would be expected. Deviation from H-W Equilibrium suggests the presence of a linked resistance gene. The results are shown in Table 4 below.

TABLE 4
Chi-Square contrast analysis between divergent phenotype tails of mapping population S6 lines.

| | simplex marker | | | | | |
|---|---|---|---|---|---|---|
| | PZE-106073001 | PUT-163a-18167596-1294 | S_54943_173 | PZE-106073260 | S_54949_671 | PZE-106073330 |
| | position AGP v4 | | | | | |
| | 132527554 | 132569643 | 132618791 | 132727834 | 132805062 | 132828360 |
| Resistant | 21 | 20 | 21 | 21 | 21 | 21 |
| tail lines | | | | | | |
| R tail obs | 30 | 32 | 34 | 38 | 38 | 38 |
| (donor alleles) | | | | | | |
| R tail exp. | 21 | 20 | 21 | 21 | 21 | 21 |
| (if | | | | | | |
| independent) | | | | | | |
| S tail lines | 15 | 15 | 15 | 15 | 15 | 15 |
| S tail obs | 8 | 4 | 0 | 0 | 0 | 0 |
| (RP alleles) | | | | | | |
| S tail | 15 | 15 | 15 | 15 | 15 | 15 |
| expected | | | | | | |
| (R obs − R | 3.9 | 7.2 | 8.0 | 13.8 | 13.8 | 13.8 |
| exp)\hat{ }2/R exp | | | | | | |
| (S obs − S | 3.3 | 8.1 | 15.0 | 15.0 | 15.0 | 15.0 |
| exp)\hat{ }2/S exp | | | | | | |
| sum Chi-Sq | 7.1 | 15.3 | 23.0 | 28.8 | 28.8 | 28.8 |

| | simplex marker | | | | | |
|---|---|---|---|---|---|---|
| | S_54956_986 | PZE-106073551 | S_54966_239 | PZE-106073623 | S_54969_513 | S_54972_725 |
| | position AGP v4 | | | | | |
| | 133038790 | 133193926 | 133317185 | 133326430 | 133421165 | 133497579 |
| Resistant | 21 | 21 | 21 | 21 | 21 | 21 |
| tail lines | | | | | | |
| R tail obs | 42 | 42 | 42 | 42 | 42 | 42 |
| (donor alleles) | | | | | | |
| R tail exp. | 21 | 21 | 21 | 21 | 21 | 21 |
| (if | | | | | | |
| independent) | | | | | | |
| S tail lines | 15 | 15 | 15 | 15 | 15 | 15 |
| S tail obs | 0 | 0 | 0 | 0 | 0 | 0 |
| (RP alleles) | | | | | | |
| S tail | 15 | 15 | 15 | 15 | 15 | 15 |
| expected | | | | | | |
| (R obs − R | 21.0 | 21.0 | 21.0 | 21.0 | 21.0 | 21.0 |
| exp)\hat{ }2/R exp | | | | | | |
| (S obs − S | 15.0 | 15.0 | 15.0 | 15.0 | 15.0 | 15.0 |
| exp)\hat{ }2/S exp | | | | | | |
| sum Chi-Sq | 36.0 | 36.0 | 36.0 | 36.0 | 36.0 | 36.0 |

| | simplex marker | | | | |
|---|---|---|---|---|---|
| | S_54973_806 | S_54975_531 | S_54977_728 | S_54978_371 | PZE-106074105 |
| | position AGP v4 | | | | |
| | 133507966 | 133535334 | 133579300 | 133595880 | 133604226 |
| Resistant | 21 | 21 | 21 | 21 | 21 |
| tail lines | | | | | |
| R tail obs | 42 | 42 | 42 | 42 | 42 |
| (donor alleles) | | | | | |
| R tail exp. | 21 | 21 | 21 | 21 | 21 |
| (if | | | | | |
| independent) | | | | | |
| S tail lines | 14 | 15 | 15 | 15 | 15 |
| S tail obs | 6 | 2 | 2 | 2 | 8 |
| (RP alleles) | | | | | |
| S tail | 14 | 15 | 15 | 15 | 15 |
| expected | | | | | |
| (R obs − R | 21.0 | 21.0 | 21.0 | 21.0 | 21.0 |
| exp)\hat{ }2/R exp | | | | | |
| (S obs − S | 4.6 | 11.3 | 11.3 | 11.3 | 3.3 |
| exp)\hat{ }2/S exp | | | | | |
| sum Chi-Sq | 25.6 | 32.3 | 32.3 | 32.3 | 24.3 |

The results from the above analysis indicate that the causal gene(s) for resistance lie between the AGP v4.0 physical map positions chr6: 129032506 and chr6: 129436586, a distance of 404,080 base pairs.

To further narrow down the mapping interval new homozygote recombinant plants (76 from 15 families) were phenotyped again in one location with two replications and three replications per recombinant plant. With four recombinant plants on the left site and three recombinant plants on the right site the new interval could be determined from marker PZE-106073330 (B73AGPv04 132828359 bp) to Affx-90423958 (B73AGPv04 133070737) (see further below Table 11).

### Example 2. Development of Stalk Splitter Apparatus

A stalk splitter apparatus was developed with the goal of more safely and efficiently splitting stalks in the field. The stalk splitter has a solid aluminum body with a hole in the middle through which the cut off stalk is passed. See, e.g., FIGS. 5 and 6. A blade is centered above the hole to split open the stalk. The blade is easily removable for sharpening. During operation, the stalk splitter is initially positioned above the corn stalk such that the top of the corn stalk fits into the hole.

Below the blade are two rollers which push in on the stalk to center it such that the cut of the variable diameter stalks is consistently in the middle. These rollers are tensioned by springs residing in channels drilled horizontally out from the center hole in the body of the splitter on either side of the rollers.

The stalk splitter cuts the corn stalk through application of downward force onto the corn stalk.

The stalk splitter device has 50 cm handles extending up from the outer top to enable the user to push down on the cut off stalk all the way to the ground while standing. The handles are shown in FIG. 3.

Throughout the examples described herein, phenotyping of corn stalks was performed by inoculating the stalks with the causal organism (e.g., Colletotrichum graminicola or Fusarium verticilliodes spores). At physiologic maturity, inoculated plants were cut off at about 0.7 m high with limb trimmers. The degree of internal rot was assessed by splitting open the stalk for visual rating.

### Example 3. Evaluation of Resistance Locus on Chromosome 4

PCR amplification was performed to amplify sequences on chromosome 4, particularly those described in U.S. Pat. No. 8,062,847, which is incorporated by reference herein in its entirety. For the genic interval covered by SEQ ID NO: 50, five overlapping amplicons were in common from the 5′ end (Appendix 1). However, Mp305 produced amplicons at the end of the interval, whereas NC262A and NC342 did not.

Several single nucleotide polymorphisms (SNPs) between Mp305 and DW1035 versus NC262a and NC342 could be identified from a comparison of amplicon sequences shown in FIG. 2. In particular, Table 5 shows ten exemplary polymorphisms between Mp305 and DW1035 vs. NC262a and NC342 within amplicons shown in FIG. 2. In two cases, SNPs result in alteration of the amino acid sequence as indicated in Table 5 in the column titled “Effect”.

TABLE 5

| | | Exon/ | | |
|---|---|---|---|---|
| SNP | Amplicon | Intron | Polymorphism | Effect |
| 1 | 2 | Intron | A → C | silent |
| 2 | 2 | Intron | G → C | silent |
| 3 | 2 | Intron | C → T | silent |
| 4 | 2 | Intron | C → A | silent |
| 5 | 2 | Intron | A → T | silent |
| 6 | 3 | Exon2 | A → G | Lysine (L) → Arginine (R) |
| 7 | 4 | Exon2 | A → G | silent |
| 8 | 4/5 | Exon2 | G → A | silent |
| 9 | 6 | Exon2 | C → A | Serine (S) → Arginine (R) |
| 10 | 6 | Exon2 | A → C | silent |

In Table 7 of U.S. Pat. No. 8,062,847, Mp305 was shown to have a C at position 1308 of SEQ ID NO: 50 as well as its resistant progeny. Other genotypes lack a fragment containing this base and are polymorphic at other positions. A novel insert is included in Mp305, its progeny DW1035, as well as in NC262A and NC342. Other genotypes tested in that patent as well as those genotyped and shown in Table 6 lack these amplicons.

It is important to note that Mp305 and DW1035 have amplicons beyond the region specified in that patent whereas NC262A and NC342 do not. NC262A and NC342 have a certain level of identity over all five amplicons. These lines possess two distinct alleles on chromosome 4 as further demonstrated in Tables 3 and 4. There are at least 10 SNPs between them in addition to having a shorter insert.

Selected SNPs were identified in allelic variants NC262A and NC342 on chromosome 4. Consensus position is determined with reference to SEQ ID NO: 50 (identical to SEQ ID NO: 1 of U.S. Pat. No. 8,062,847). Using the published sequences, the bases delineated Table 7 of U.S. Pat. No. 8,062,847 were analyzed. The data is shown in Table 6 below, with “NA” denoting “Not Amplified”. Primer sequences for detecting the SNPs in chromosome 4 are shown in Table 7.

TABLE 6

| | C0060- | Consensus position | | | | | | | | | | |
|---|---|---|---|---|---|---|---|---|---|---|---|---|
| Sample | 02 | 413 | 958 | 971 | 1099 | 1154 | 1250 | 1308 | 1607 | 2001 | 2598 | 3342 |
| Mp305 | Rcg1 | A:A | A:A | G:G | C:C | C:C | A:A | C:C | A:A | A:A | G:G | C:C |
| DW1035 | Rcg1 | A:A | A:A | G:G | C:C | C:C | A:A | C:C | A:A | A:A | G:G | C:C |
| NC262A | Rcg1 | C:C | C:C | C:C | T:T | A:A | T:T | C:C | G:G | G:G | A:A | A:A |
| NC342 | Rcg1 | C:C | C:C | C:C | T:T | A:A | T:T | C:C | G:G | G:G | A:A | A:A |
| GEMN- | NA | NA | NA | NA | NA | NA | NA | NA | NA | NA | NA | NA |
| 0117 | | | | | | | | | | | | |
| GEMS- | NA | NA | NA | NA | NA | NA | NA | NA | NA | NA | NA | NA |
| 0016 | | | | | | | | | | | | |
| blank | NA | NA | NA | NA | NA | NA | NA | NA | NA | NA | NA | NA |
| SNP | | | 1 | 2 | 3 | 4 | 5 | | 6 | 7 | 8 | 9 |
| acc. to | | | | | | | | | | | | |
| Table 3 | | | | | | | | | | | | |

TABLE 7
Markers to detect SNPs of Table 6: Chromosome 4 primers

| SNP_id | Primer_AlleleX | Primer_AlleleY | Primer_Common |
|---|---|---|---|
| Rcg1_SE | GAAGGTGACCAAGTT | GAAGGTCGGAGTCAACG | GCGACCGTACAGTCCGA |
| Q1413 | CATGCTATCGCTTCTT | GATTCGCTTCTTGTACCA | CGAAA |
| | GTACCATGTGACCA | TGTGACCC | (SEQ ID NO: 60) |
| | (SEQ ID NO: 58) | (SEQ ID NO: 59) | |
| Rcg1_SE | GAAGGTGACCAAGTT | GAAGGTCGGAGTCAACG | GAATAAAATAACGGTGA |
| Q11099 | CATGCTTTGGAAAAG | GATTCTTTGGAAAAGCG | GAAATGTATGCAA |
| | CGGTAGTGTTTTGAC | GTAGTGTTTTGAT | (SEQ ID NO: 63) |
| | (SEQ ID NO: 61) | (SEQ ID NO: 62) | |
| Rcg1_SE | GAAGGTGACCAAGTT | GAAGGTCGGAGTCAACG | CTTCCATGCCCAAAACA |
| Ql_1250 | CATGCTCAATCAGTG | GATTCAATCAGTGTGTC | TGTCCGAT |
| | TGTCTGATCTCAAAG | TGATCTCAAAGAA | (SEQ ID NO: 66) |
| | AT | (SEQ ID NO: 65) | |
| | (SEQ ID NO: 64) | | |
| Rcg1_SE | GAAGGTGACCAAGTT | GAAGGTCGGAGTCAACG | GCTCTGAGTGTTCGTCA |
| Q11607 | CATGCTCTTCTAACGG | GATTCTCTTCTAACGGTT | CTAAGTCTT |
| | TTATGTGCACAA | ATGTGCACAG | (SEQ ID NO: 69) |
| | (SEQ ID NO: 67) | (SEQ ID NO: 68) | |
| Rcg1_SE | GAAGGTGACCAAGTT | GAAGGTCGGAGTCAACG | GCGATGGTTAGCAGAGG |
| Ql_2001 | CATGCTTGAAGTTTTC | GATTGAAGTTTTCACTA | GATTTGTA |
| | ACTAGATGAAGGCTG | GATGAAGGCTGC | (SEQ ID NO: 72) |
| | T | (SEQ ID NO: 71) | |
| | (SEQ ID NO: 70) | | |
| Rcg1_SE | GAAGGTGACCAAGTT | GAAGGTCGGAGTCAACG | GTCTCTAAGTTACTCTGT |
| Q12598 | CATGCTATAAAATCA | GATTGATAAAATCAAGA | CCAGTTAAGAA |
| | AGATAACAAGTGACA | TAACAAGTGACATGCAA | (SEQ ID NO: 75) |
| | TGCAG | (SEQ ID NO: 74) | |
| | (SEQ ID NO: 73) | | |
| Rcg1_SE | GAAGGTGACCAAGTT | GAAGGTCGGAGTCAACG | AGCAACAGAACAGTCTA |
| Ql_3342 | CATGCTGTTGGTCAA | GATTGTTGGTCAACTCA | CAGCCTTAATTT |
| | CTCATCTGATGAAAG | TCTGATGAAAGA | (SEQ ID NO: 78) |
| | C | (SEQ ID NO: 77) | |
| | (SEQ ID NO: 76) | | |

The data in Table 6 shows that the resistance alleles from NC262A and NC342 are the same. These resistance alleles are allelic to that from Mp305, but not identical in state owing to its truncated length and many SNPs. The alleles are alike only at position 1308 using these markers.

Selection of inbreds with ASR resistance was effective via marker assisted backcrossing (MABC) for the NC262A or NC342 allele using KASP markers outside this genic region.

### Example 4. Evaluation of Genes in Resistance Locus on Chromosome 6

As described above, the region on chromosome 6 has been fine mapped. Table 8 shows candidate genes for the resistance phenotype have been identified in the fine mapped region linked closely with the trait on chromosome 6.

TABLE 8
Candidate Genes in the ASR Locus on Chromosome 6

| Gene | | | | |
|---|---|---|---|---|
| in | | | | Gene |
| region | Identifier | v4 interval | Gene name | symbol |
| A | Zm00001d037636 | Chr6: 132563692 | Cellulose synthase 2 | cesa2 |
| | | . . . 132569944 | | |
| B | Zm00001d037645 | Chr6: 132762227 | Calcium dependent protein | CDPK3 |
| | | . . . 132764738 | kinase 3 | |
| C | Zm00001d037648 | Chr6: 132796567 | NB-ARC LRR13 disease | Rpp13 |
| | | . . . 132805878 | resistance protein | |
| D | Zm00001d037666 | Chr6: 133532459 | Homology to serine/threonine | GSO1 |
| | | . . . 133535392 | protein kinase | |
| E | Zm00001d037682 | Chr6: 133908212 | Leucine-rich repeat extensin- | LRX4 |
| | | . . . 133909537 | like protein 4 | |
| F | Zm00001d037635 | Chr6: 132525481 | wall-associated receptor protein | |
| | | . . . 132529995 | kinase family protein | |
| G | Zm00001d037637 | Chr6: 132582694 | wall-associated receptor protein | |
| | | . . . 132586993) | kinase family protein | |
| H | Zm00001d037640 | Chr6: 132629007 | Adenine nucleotide transporter | |
| | | . . . 132634967 | BT1 chloroplastic/mitochondrial | |
| I | Zm00001d037642 | Chr6: 132669023 | | |
| | | . . . 132672000 | | |
| J | Zm00001d037643 | Chr6: 132723336 | phospholipase D4 | |
| | | . . . 132728573 | | |
| K | Zm00001d037647 | Chr6: 132784055 | hydroxyproline-rich | |
| | | . . . 132787415 | glycoprotein family protein | |
| L | Zm00001d037650 | Chr6: 132828573 | | |
| | | . . . 132836981 | | |
| M | Zm00001d037651 | Chr6: 133037545 | Subtilisin-like serine | |
| | | . . . 133043210 | endopeptidase family protein | |
| N | Zm00001d037652 | Chr6: 133048475 | Inositol-3-phosphate synthase | |
| | | . . . 133050067 | isozyme 1 | |
| O | Zm00001d037653 | Chr6: 133069804 | Xylanase inhibitor protein 1 | |
| | | . . . 133070751 | | |
| P | Zm00001d037656 | Chr6: 133140745 | Xylanase inhibitor protein 1 | |
| | | . . . 133141716 | | |
| Q | Zm00001d037658 | Chr6: 133192852 | | |
| | | . . . 133194981 | | |
| R | Zm00001d037659 | Chr6: 133275793 | Phosphoenolpyruvate/phosphate | |
| | | . . . 133278869 | translocator 2 chloroplastic | |
| S | Zm00001d037661 | Chr6: 133296885 | Serine/threonine-protein kinase | |
| | | . . . 133300907 | | |
| T | Zm00001d037663 | Chr6: 133420435 | NADH-ubiquinone | |
| | | . . . 133424501 | oxidoreductase 10.5 kDa subunit | |
| U | Zm00001d037668 | Chr6: 133555104- | NEP-interacting protein 1 | |
| | | 133557311 | | |
| V | Zm00001d037672 | Chr6: 133574540 | leucine-rich repeat receptor | |
| | | . . . 133578520 | protein kinase family protein | |
| W | Zm00001d037674 | Chr6: 133714246 | Alliin lyase | |
| | | . . . 133717685 | | |
| X | Zm00001d037675 | Chr6: 133723502 | Actin-like ATPase superfamily | |
| | | . . . 133727955 | protein | |
| Y | AGP9L | Chr6: 132,890,926 | Arabinogalactan protein 9-like | |
| | | . . . 132,891,357 | | |

The involvement of the genes can be verified by several means. A mapping population having been derived through selfing generated a number of pairs of near isogenic lines having the donor or susceptible parent fragment differing between S5 sisters. An orthogonal comparison of the contrasting phenotypes of approximately 16 such pairs will further confirm which fragment contains the causative gene. Further, expression analysis by quantitative PCR for the gene would reveal differential expression.

A cDNA library generated from RNA extracted from the infected nodal regions would be used. One such gene is calcium dependent protein kinase (CDPK).

Constitutive or induced resistance is also shown by knocking out the putative resistance gene by mutagenesis, i.e. TILLING (Targeted Induced Local Lesions in Genomes) (Gilchrist, E., et al., TILLING is an effective reverse genetics technique for C. elegans. BMC Genomics, 2006, 7(1), 262.) In such cases, a stop codon, frame shift, or non-synonymous mutation renders the gene product ineffective.

On chromosome 6, patent publication WO/2015088970, incorporated by reference herein in its entirety, describes the selection for ASR resistance associated with the haplotypes described herein. The bases delineated in Table 6 above were analyzed. The resistance sources uncovered on chromosome 6 that are shown in Table 9 are polymorphic at the base designated in bold. An additional SNP was identified in allelic variant NC262A that was not described in WO2015/088970, and is shown in Table 9 below. The locus on chromosome 6 shown in Table 9 (C16759-001-K1) is a distinct allele not described in WO2015/088970.

TABLE 9
Locus SNP markers on Chromosome
6 Compared to WO 2015/088970

| Locus SNP | NC262A | WO 2015088970 |
|---|---|---|
| C12305-001-K1 | C:C | C:C |
| C12307-001-K1 | C:C | C:C |
| C16759-001-K1 | G:G | A:A |
| C16760-001-K1 | G:G | G:G |
| C12314-001-K1 | A:A | A:A |

Primer sequences for detecting the SNPs are shown in Table 10.

TABLE 10
Primer Sequences for Detecting the SNPs in Table 9

| Code | Primer_AlleleX | Primer_AlleleY | Primer_Common |
|---|---|---|---|
| C12305-001-K1 | GAAGGTGACCAAGTT | GAAGGTCGGAGTCAA | ACACACCTTCAATAC |
| | CATGCTAACAGAATC | CGGATTGAACAGAAT | CCTCCTCGAT |
| | TTCAGCTCCCATCTTC | CTTCAGCTCCCATCTT | (SEQ ID NO: 81) |
| | (SEQ ID NO: 79) | T | |
| | | (SEQ ID NO: 80) | |
| C12307-001-K1 | GAAGGTGACCAAGTT | GAAGGTCGGAGTCAA | CAATCTTCAATGCCA |
| | CATGCTTCTTTTYGCC | CGGATTCTTCTTTTYG | ACACCACTCCAT |
| | TCCATTTTCGCC | CCTCCATTTTCGCA | (SEQ ID NO: 84) |
| | (SEQ ID NO: 82) | (SEQ ID NO: 83) | |
| C16759-001-K1 | GAAGGTGACCAAGTT | GAAGGTCGGAGTCAA | CGTTGAGATGCTTTCA |
| | CATGCTAGAGTTCCCC | CGGATTGAGTTCCCC | TAATGATCAAGGTA |
| | ATAATTATGCTGATGA | ATAATTATGCTGATGG | (SEQ ID NO: 87) |
| | (SEQ ID NO: 85) | (SEQ ID NO: 86) | |
| C16760-001-K1 | GAAGGTGACCAAGTT | GAAGGTCGGAGTCAA | TTGCCAGAGAAGTAT |
| | CATGCTAGACTGATT | CGGATTAGACTGATT | GGTCTTACTCTTA |
| | GATCATTTTAGGGGG | GATCATTTTAGGGGG | (SEQ ID NO: 91) |
| | A | G | |
| | (SEQ ID NO: 88) | (SEQ ID NO: 89) | |
| C12314-001-K1 | GAAGGTGACCAAGTT | GAAGGTCGGAGTCAA | AAAAAGTCAGTACAG |
| | CATGCTCATCCAATG | CGGATTATCCAATGG | ACTTGTGGRACAT |
| | GCATTAAGTGACCTC | CATTAAGTGACCTCG | (SEQ ID NO: 93) |
| | A | (SEQ ID NO: 92) | |
| | (SEQ ID NO: 91) | | |

The involvement of the candidate genes in anthracnose stalk rot resistance can be verified by several means. A mapping population having been derived through selfing, generated a number of pairs of near isogenic lines having the donor or susceptible parent fragment differing between S5 sisters. An orthogonal comparison of the contrasting phenotypes of approximately 16 such pairs will further confirm which fragment contains the causative gene. Further, expression analysis by quantitative PCR for the gene would reveal differential expression. Constitutive or induced resistance can also be shown by knocking out the putative resistance gene by mutagenesis, i.e. TTLLING (Targeted Induced Local Lesions in Genomes) (Gilchrist, E., et al., TILLING is an effective reverse genetics technique for C. elegans. BMC Genomics, 2006, 7(1), 262.) In such cases, a stop codon, frame shift, or non-synonymous mutation renders the gene product ineffective.

Further mapping of the QTL on chromosome 6 was performed by the identification of additional recombinants as shown in Table 11. Based on these recombinants, the list of candidate genes for anthracnose stalk rot resistance has been narrowed to two genes: Zm00001d037650 and Zm00001d037651, identified according to the annotation of B73AGPv4.

TABLE 11
Anthracnose stalk rot Resistance Candidate Genes on Chromosome 6

| | | position | | |
|---|---|---|---|---|
| | | B73AGPv04 | B73AGPv05 | NC262A |
| Recombinant- | Marker left - | 132828359 | 139646631 | MA_NC262A_v2.contig81: 29672136 |
| interval new | PZE- | | | |
| | 106073330 | | | |
| Recombinant- | Marker right - | 133070737 | 139889078 | MA_NC262A_v2.contig81: 29819546 |
| interval new | Affx-90423958 | | | |

The positions of the two candidate genes on chromosome 6 are shown in Table 12 according to different versions of the maize reference genome. For example, Table 12 shows the annotation of these in the older genome reference B73AGPv02 (identifiers: GRMZM2G002656 and GRMZM2G145589) and most recent reference B73AGPv5 (identifiers: Zm00001e0311 094 and Zm00001e031197.

TABLE 12
Annotation of Anthracnose Resistance Candidate Genes

| | B73AGPv02 | B73AGPv04 | B73AGPv05 | NC262A |
|---|---|---|---|---|
| Position | 6: 128823409 . . . | 6: 132828573 . . . | 6: 139644733 . . . | MA_NC262A_v2.contig81: |
| | 128833681 | 132832542 | 139655572 | 29668461 . . . 29683871 |
| Gene | GRMZM2G002656 | Zm00001d037650 | Zm00001e031194 | ZmNC262Av2c_OGOO1508HC.1 |
| ID | | | | |
| Position | 6: 129031310 . . . | 6: 133037545 . . . | 6: 139855686 . . . | MA_NC262A_v2.contig81: |
| | 129036927 | 133043210 | 139861571 | 29804742-29814143 |
| Gene | GRMZM2G145589 | Zm00001d037651 | Zm00001e031197 | ZmNC262Av2c_OGOO1512HC.1 |
| ID | | | | |

It was noted that the sequence annotation of these loci varies between the B73AGPv04 and B73AGPv05 sequences. The genomic sequence for candidate gene Zm00001e031194 in the NC262A genotype (ZmNC262Av2c_OGOO1508HC.1) is provided in SEQ ID NO: 266. The putative cDNA for candidate gene Zm00001e031194 in the NC262A genotype is provided in SEQ ID NO: 267 (ZmNC262Av2c_OGOO1508HC.1). The protein encoded by SEQ ID NO: 267 is provided in SEQ ID NO: 268. The genomic sequence for candidate gene for candidate gene Zm00001e031197 in the NC262A genotype (ZmNC262Av2c_OGOO1512HC.1) is provided in SEQ ID NO:269. The cDNA for candidate gene Zm00001e031197 in the NC262A genotype is provided in SEQ ID NO: 270 (ZmNC262Av2c_OGOO1512HC.1). The protein encoded by SEQ ID NO: 270 is provided in SEQ ID NO: 271.

Both candidate genes Zm00001e031194 and Zm00001e031197 are located on a contig derived from NC262A called MA_NC262A_v2.contig81. The complete genomic sequence between the new marker positions PZE-106073330-Affx-90423958 is shown in SEQ ID NO: 272. This contig derived from source NC262A can be used as target for markers for screening on resistance locus. Table 13 lists polymorphisms which are usable to identify anthracnose stalk rot resistance locus and follow the anthracnose stalk rot resistance locus during breeding selection numbered according to the B73AGPv04 genome sequence.

TABLE 13
Useful Polymorphisms

| | | | Variant | |
|---|---|---|---|---|
| | | | Nucleotide(s) | marker |
| Position | Chromo- | Reference | in | SEQ ID |
| B73_V4 | some | Sequence | NC262A | NO: |
| 132836954 | 6 | G | A | 273 |
| 132836944 | 6 | C | T | 274 |
| 132836869 | 6 | G | A | 275 |
| 132836849 | 6 | G | T | 276 |
| 132836845 | 6 | C | T | 277 |
| 132836840 | 6 | C | G | 278 |
| 132836830 | 6 | G | T | 279 |
| 132836810 | 6 | G | A | 280 |
| 132836805 | 6 | AG | CA | 281; 282 |
| 132836802 | 6 | ATC | --- | 283; 284 |
| 132836799 | 6 | ATT | --- | 283; 284 |
| 132836791 | 6 | G | A | 285 |
| 132836787 | 6 | ATA | CTG | 286; 287 |
| 132836781 | 6 | AT | GA | 288; 289 |
| 132836776 | 6 | G | T | 290 |
| 132836767 | 6 | GA | AG | 291; 292 |
| 132836762 | 6 | G | A | 293 |
| 132836679 | 6 | “------G” | CGCCAAA | 294; 295 |
| 132836678 | 6 | T | A | 296 |
| 132836670 | 6 | T | G | 297 |
| 132836667 | 6 | G | T | 298 |
| 132836665 | 6 | T | A | 299 |
| 132836662 | 6 | T | C | 300 |
| 132836660 | 6 | G | T | 301 |
| 132836651 | 6 | A | G | 302 |
| 132836643 | 6 | AAT | --- | 303; 304 |
| 132836636 | 6 | GCCATG | ------ | 305; 306 |
| 132836622 | 6 | CA | AG | 307; 308 |
| 132836620 | 6 | GA | AC | 309; 310 |
| 132836612 | 6 | C | G | 311 |
| 132836603 | 6 | A | G | 312 |
| 132836591 | 6 | G | C | 313 |
| 132836586 | 6 | G | A | 314 |
| 132836580 | 6 | T | G | 315 |
| 132836069 | 6 | G | A | 316 |
| 132836063 | 6 | G | A | 317 |
| 132836061 | 6 | A | G | 318 |
| 132836056 | 6 | T | G | 319 |
| 132836050 | 6 | T | C | 320 |
| 132836047 | 6 | ACC | CGT | 321; 322 |
| 132836034 | 6 | T | G | 323 |
| 132836031 | 6 | T | G | 324 |
| 132836019 | 6 | A | T | 325 |
| 132836008 | 6 | C | G | 326 |
| 132835978 | 6 | C | G | 327 |
| 132835910 | 6 | A | G | 328 |
| 132835851 | 6 | A | G | 329 |
| 132835819 | 6 | T | C | 330 |
| 132835803 | 6 | A | G | 331 |
| 132835793 | 6 | A | T | 332 |
| 132835788 | 6 | A | C | 333 |
| 132835781 | 6 | T | A | 334 |
| 132835779 | 6 | G | A | 335 |
| 132835776 | 6 | A | T | 336 |
| 132835746 | 6 | A | G | 337 |
| 132835729 | 6 | C | A | 338 |
| 132835728 | 6 | G | C | 339 |
| 132835722 | 6 | ------ | GACATC | 340; 341 |
| 132835713 | 6 | A | T | 342 |
| 132835708 | 6 | GAA | --- | 343; 344 |
| 132835705 | 6 | ATA | --- | 343; 344 |
| 132835702 | 6 | GAT | --- | 343; 344 |
| 132835693 | 6 | A | G | 345 |
| 132835681 | 6 | C | T | 346 |
| 132835671 | 6 | A | C | 347 |
| 132835666 | 6 | A | T | 348 |
| 132835271 | 6 | G | A | 349 |
| 132835267 | 6 | AC | GG | 350; 351 |
| 132835147 | 6 | G | T | 352 |
| 132835031 | 6 | T | C | 353 |
| 132834960 | 6 | G | A | 354 |
| 132834951 | 6 | G | A | 355 |
| 132834946 | 6 | GA | AC | 356; 357 |
| 132834942 | 6 | G | A | 358 |
| 132834929 | 6 | T | A | 359 |
| 132834927 | 6 | A | T | 360 |
| 132834924 | 6 | T | A | 361 |
| 132834919 | 6 | G | C | 362 |
| 132834908 | 6 | G | T | 363 |
| 132834899 | 6 | C | A | 364 |
| 132834754 | 6 | G | C | 365 |
| 132834753 | 6 | A | C | 366 |
| 132834748 | 6 | G | C | 367 |
| 132834721 | 6 | G | A | 368 |
| 132834632 | 6 | C | A | 369 |
| 132834622 | 6 | T | C | 370 |
| 132834615 | 6 | C | G | 371 |
| 132834602 | 6 | T | C | — |
| 132834589 | 6 | A | G | 372 |
| 132834581 | 6 | C | T | 373 |
| 132834577 | 6 | T | A | 374 |
| 132834569 | 6 | T | G | 375 |
| 132834539 | 6 | A | C | 376 |
| 132834532 | 6 | AGT | GAG | 377; 378 |
| 132834494 | 6 | C | T | 379 |
| 132834431 | 6 | C | T | 380 |
| 132834389 | 6 | A | G | 381 |
| 132834208 | 6 | C | T | 382 |
| 132831963 | 6 | A | T | 383 |
| 132831924 | 6 | T | G | 384 |
| 132831915 | 6 | G | A | 385 |
| 132831839 | 6 | C | G | 386 |
| 132829096 | 6 | C | T | 387 |
| 132828997 | 6 | T | A | 388 |
| 132828958 | 6 | A | G | 389 |
| 132828950 | 6 | C | G | 390 |
| 132828867 | 6 | T | C | 391 |
| 132827606 | 6 | G | A | 392 |
| 132827596 | 6 | ----- | AGTTC | 393; 394 |
| | | ----- | ATAAT | |
| | | ----- | AAAGT | |
| | | ----- | GATAG | |
| | | ---- | AGTT | |
| | | | (SEQ ID | |
| | | | NO: | |
| | | | 414) | |
| 132827573 | 6 | C | T | 395 |
| 132827566 | 6 | A | G | 396 |
| 132827549 | 6 | C | A | 397 |
| 132827545 | 6 | ACG | TAT | 398; 399 |
| 132827302 | 6 | A | C | 400 |
| 132826983 | 6 | C | T | 401 |
| 132826940 | 6 | C | T | 402 |
| 133040380 | 6 | C | T | 403 |
| 133040382 | 6 | A | C | 404 |
| 133040760 | 6 | C | T | 405 |

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS OF THE DISCLOSURE

Notwithstanding the appended claims, the disclosure sets forth the following numbered embodiments:

1. A process of identifying a maize plant that displays enhanced resistance to anthracnose stalk rot, the process comprising detecting in the maize plant
a. the presence of at least two markers at a resistance locus on chromosome 6 comprising a “G” at C16759-001-K1 and one of the following single nucleotide polymorphisms:
   a “C” at C12305-001-K1,
   a “C” at C12307-001-K1,
   a “G” at C16760-001-K1,
   an “A” at C12314-001-K1; and/or
b. the presence of at least one marker at a resistance locus on chromosome 6 comprising
at least one of the variant nucleotide polymorphisms recited in Table 13, wherein the at least two markers of (a) and/or the at least one marker of (b) are closely linked to and associated with the resistance locus on chromosome 6.
   2. The process of embodiment 1, wherein the resistance locus on chromosome 6 is located on a chromosomal interval between makers PZE-106066805 and PZE-106075546.
   3. The process of embodiment 1, wherein the resistance locus on chromosome 6 is located on a chromosomal interval between 139646631 and 139889078 numbered according to the B73AGPv05 genome sequence.
   4. The process of embodiment 1, wherein the resistance locus of chromosome 6 comprises SEQ ID NO: 272, or a fragment thereof.
   5. The process of embodiment 1, wherein the resistance locus on chromosome 6 comprises one or more nucleotide sequences selected from the group consisting of
i. a nucleotide sequence of SEQ ID NO: 266 or 269,
ii. a nucleotide sequence having a coding sequence of SEQ ID NO: 267 or 270,
iii. a nucleotide sequence which is complementary to a sequence from i. or ii.,
iv. a nucleotide sequence which has at least 80% identity with a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 268 or 271, or
viii. a nucleotide sequence which encodes for a protein comprising a sequence with at least 80% identity to the sequence of SEQ ID NOs: 268 or 271.
   6. The process of embodiment 1, wherein the resistance locus on chromosome 6 is derived from NC262A.
   7. The process of embodiment 1, the process comprising detecting in the maize plant the presence or absence of at least one allele at the resistance locus on chromosome 6 from embodiment 1 (b).
   8. The process of any one of embodiments 1-7, wherein the at least one marker at the resistance locus on chromosome 6 detects a “G” at C16759-001-K1.
   9. The method of any one of embodiments 1 to 8, wherein the presence or absence of at least one nucleotide polymorphism is detected by polymerase chain reaction amplification of a nucleic acid present in the maize plant with a primer configured to specifically amplify a nucleic acid sequence comprising one or more of the nucleotide polymorphisms.
   10. The method of embodiment 9, wherein the nucleotide polymorphisms are selected from the group consisting of the variant nucleotides recited in Table 13.
   11. The method of any one of embodiments 1 to 10, wherein the presence or absence of the allele comprising a “G” at C16759-001-K1 is detected by polymerase chain reaction amplification of a nucleic acid present in the maize plant with a primer configured to specifically amplify a nucleic acid sequence of the allele.
   12. The method of embodiment 11, wherein the primer comprises the sequence AATTATGCTGATGA (SEQ ID NO: 413).
   13. A process of selecting a maize plant with anthracnose stalk rot resistance, the process comprising identifying the maize plant according to the process of any one of embodiments 1 to 12, and selecting the maize plant as having anthracnose stalk rot resistance if the presence or absence of the at least one marker at the resistance locus on chromosome 6 is detected.
   14. The process of embodiment 13, further comprising selecting the maize plant that comprises at least one additional marker allele that is closely linked to and associated with the nucleotide polymorphism(s).
   15. The process of embodiment 14, wherein the additional marker allele is linked to the single nucleotide polymorphism by no more than 2 cM on a single meiosis based genetic map.
   16. The process of any one of embodiments 13 to 15, further comprising selecting the maize plant that comprises at least one additional marker allele that is linked to and associated with the allele comprising a “G” at C16759-001-K1.
   17. The process of embodiment 16, wherein the additional marker allele is linked to the allele comprising a “G” at C16759-001-K1 by no more than 2 cM on a single meiosis based genetic map.
   18. The process of any one of embodiments 13 to 15, further comprising selecting the maize plant that comprises at least one additional marker allele that is linked to and associated with the allele comprising a variant nucleotide polymorphism recited in Table 13.
   19. The process of any one of embodiments 1 to 17, further comprising backcrossing the identified maize plant with another maize plant, preferably comprising backcrossing the resistance locus on chromosome 6 into a genotype which is not NC262A.
   20. A method of introgressing an allele associated with anthracnose stalk rot resistance into a maize plant, the method comprising:
a. screening a population with a nucleic acid assay for the detection of at least one marker at a resistance locus on chromosome 6 comprising:
   (i) the following single nucleotide polymorphisms
      a “C” at C12305-001-K1,
      a “C” at C12307-001-K1,
      a “G” at C16759-001-K1,
      a “G” at C16760-001-K1,
      an “A” at C12314-001-K1; and/or
   (ii) one or more of the variant nucleotide polymorphisms recited in Table 13; and
b. selecting from the population at least one maize plant comprising the resistance locus on chromosome 6 or comprising a “G” at C16759-001-K1 and/or one or more of the variant nucleotide polymorphisms recited in Table 13; and
c. crossing the at least one maize plant to a second maize plant;
d. evaluating progeny plants for the presence of the “G” at C16759-001-K1 and/or one or more of the variant nucleotide polymorphisms recited in Table 13; and
e. selecting progeny plants possessing the “G” at C16759-001-K1 and/or one or more of the variant nucleotide polymorphisms recited in Table 13.
   21. The method of embodiment 20, wherein the at least one marker is located within 5 cM of the “G” at C16759-001-K1.
   22. The method of embodiment 20, wherein the at least one marker is located within 1 cM of the “G” at C16759-001-K1.
   23. The method of embodiment 20, wherein the resistance locus on chromosome 6 is located on a chromosomal interval between makers PZE-106066805 and PZE-106075546.
   24. The method of embodiment 20, wherein the resistance locus on chromosome 6 is located on a chromosomal interval between 139646631 and 139889078 numbered according to the B73AGPv05 genome sequence.
   25. The method of embodiment 20, wherein the resistance locus of chromosome 6 comprises SEQ ID NO: 272, or a fragment thereof.
   26. The method of embodiment 20, wherein the resistance locus on chromosome 6 comprising one or more nucleotide sequences selected from the group consisting of
i. a nucleotide sequence of SEQ ID NO:266 or 269,
ii. a nucleotide sequence having a coding sequence of SEQ ID NO: 267 or 270,
iii. a nucleotide sequence which is complementary to a sequence from i. or ii.,
iv. a nucleotide sequence which has at least 80% identity with a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NOs: 268 or 271, or
viii. a nucleotide sequence which encodes for a protein comprising a sequence with at least 80% sequence identity to the sequence of SEQ ID NO: 268 or 271.
   27. The method of embodiment 20, wherein the resistance locus on chromosome 6 is derived from NC262A.
   28. A method of selecting a maize plant that displays resistance to anthracnose stalk rot, the method comprising:
a. obtaining a first maize plant that comprises within its genome a haplotype comprising one or more of the following single nucleotide polymorphisms
   a “C” at C12305-001-K1,
   a “C” at C12307-001-K1,
   a “G” at C16759-001-K1,
   a “G” at C16760-001-K1,
   an “A” at C12314-001-K1; and/or
one or more of a variant nucleotide polymorphism recited in Table 13; and
b. crossing the first maize plant to a second maize plant;
c. evaluating progeny plants for the haplotype in a. or at least one marker allele linked to and associated with the haplotype in b.; and
d. selecting progeny plants that possess the haplotype in a.
   29. The method of embodiment 28, wherein the first maize plant is obtained in (a) that comprises within its genome a haplotype comprising one or more of a “G” at C16759-001-K1, or a variant nucleotide polymorphism recited in Table 13.
   30. A nucleic acid molecule comprising one or more nucleotide sequences selected from the group consisting of
i. a nucleotide sequence of SEQ ID NO: 266, 267, 269, 270 or 272,
ii. a nucleotide sequence having a coding sequence of SEQ ID NO: 212 or 215,
iii. a nucleotide sequence which is complementary to a sequence from i. or ii.,
iv. a nucleotide sequence with at least 80% identity to a sequence from i., ii. or iii.,
v. a nucleotide sequence which differs from a nucleic acid sequence according to i., ii. or iii. depending on the degeneracy of the genetic code,
vi. a nucleotide sequence which hybridizes with a nucleic acid sequence according to i., ii. or iii. under stringent conditions,
vii. a nucleotide sequence which encodes for a protein comprising the sequence of SEQ ID NO: 268 or 271, or
viii. a nucleotide sequence which encodes for a protein comprising a sequence with at least 80% identity to the sequence of SEQ ID NOs: 268 or 271.
   31. An expression cassette comprising the nucleic acid molecule of embodiment 30 operatively linked to heterologous regulatory element, preferably to a heterologous promoter.
   32. A method for conferring or increasing resistance to anthracnose stalk rot in a maize plant, the method comprising the following steps:
(a) introducing or introgressing into at least one cell of a maize plant the nucleic acid molecule of embodiment 30;
(b) optionally regenerating or growing a plant from the at least one cell, and
(c) causing expression of the nucleic acid molecule in the plant.
   33. A method for manufacturing a maize plant having anthracnose stalk rot resistance, comprising the following steps:
(a) introducing or introgressing into at least one cell of a maize plant the nucleic acid molecule of embodiment 30, or the expression cassette of embodiment 31; or
(b.1) introducing of a site-directed nuclease and a repair matrix into at least one cell of a maize plant, wherein the site-directed nuclease is able to generate at least one double-strand break of the DNA in the genome of the at least one cell and the repair matrix comprises the nucleic acid molecule of embodiment 30 or a fragment thereof,
(b.2) cultivation of the at least one cell of (b.1) under conditions that allow a homology-directed repair or a homologous recombination, wherein the nucleic acid molecule is integrated from the repair matrix into the genome of the maize plant; and
(c) obtaining the plant having anthracnose stalk rot resistance from the at least one cell.
   34. The method according to embodiment 33, wherein the site-directed nuclease comprises a zinc-finger nuclease, a transcription activator-like effector nuclease, a CRISPR/Cas system, including a CRISPR/Cas9 system, a CRISPR/Cpf1 system, a CRISPR/CasX system, a CRISPR/CasY system, an engineered homing endonuclease, and a meganuclease, and/or any combination, variant, or catalytically active fragment thereof.
   35. A maize plant identified according to the process of any one of embodiments 1-19, or manufactured according to the method of embodiment 33 or embodiment 34.
   36. A maize plant comprising a resistance locus associated with anthracnose stalk rot resistance, wherein the maize plant is prepared by a process comprising introgressing the resistance locus into the maize plant according to the method of any one of embodiments 20-27.
   37. A maize plant comprising a resistance locus associated with anthracnose stalk rot resistance, wherein the maize plant is prepared by a process comprising introgressing the nucleic acid molecule of embodiment 30 into the maize plant.
   38. A maize plant comprising a resistance locus associated with anthracnose stalk rot resistance, wherein the maize plant is prepared by a process comprising introducing the nucleic acid molecule of embodiment 30 into the maize plant.
   39. A seed or plant part of the maize plant of any one of embodiments 35-38.

What is claimed is:

1. A process of identifying a maize plant that displays enhanced resistance to anthracnose stalk rot, the process comprising detecting in the maize plant
a. the presence of at least two markers at a resistance locus on chromosome 6 comprising a “G” at C16759-001-K1 and one of the following single nucleotide polymorphisms:
a “C” at C12305-001-K1,
a “C” at C12307-001-K1,
a “G” at C16760-001-K1,
an “A” at C12314-001-K1; and/or
b. the presence of at least one marker at a resistance locus on chromosome 6 comprising
at least one of the variant nucleotide polymorphisms recited in Table 13,
wherein the at least two markers of (a) and/or the at least one marker of (b) are closely linked to and associated with the resistance locus on chromosome 6.

2. The process of claim 1, wherein the resistance locus on chromosome 6 is located on a chromosomal interval between makers PZE-106066805 and PZE-106075546.

3. The process of claim 1, wherein the resistance locus on chromosome 6 is located on a chromosomal interval between 139646631 and 139889078 numbered according to the B73AGPv05 genome sequence.

4. The process of claim 1, wherein the resistance locus of chromosome 6 comprises SEQ ID NO: 272, or a fragment thereof.

5. The process of claim 1, wherein the resistance locus on chromosome 6 comprises one or more nucleotide sequences selected from the group consisting of
ix. a nucleotide sequence of SEQ ID NO: 266 or 269,
x. a nucleotide sequence having a coding sequence of SEQ ID NO: 267 or 270,
xi. a nucleotide sequence which is complementary to a sequence from i. or ii.,
xii. a nucleotide sequence which has at least 80% identity with a sequence from i., ii. or iii.,
xiii. a nucleotide sequence which differs from a nucleic acid sequence according to i., ii. or iii. depending on the degeneracy of the genetic code,
xiv. a nucleotide sequence which hybridizes with a nucleic acid sequence according to i., ii. or iii. under stringent conditions,
xv. a nucleotide sequence which encodes for a protein comprising the sequence of SEQ ID NO: 268 or 271, or
xvi. a nucleotide sequence which encodes for a protein comprising a sequence with at least 80% identity to the sequence of SEQ ID NOs: 268 or 271.

6. The process of claim 1, wherein the resistance locus on chromosome 6 is derived from NC262A.

7. The process of claim 1, the process comprising detecting in the maize plant the presence or absence of at least one allele at the resistance locus on chromosome 6 from claim 1 (b).

8. The process of any one of claims 1-7, wherein the at least one marker at the resistance locus on chromosome 6 detects a “G” at C16759-001-K1.

9. The method of any one of claims 1 to 8, wherein the presence or absence of at least one nucleotide polymorphism is detected by polymerase chain reaction amplification of a nucleic acid present in the maize plant with a primer configured to specifically amplify a nucleic acid sequence comprising one or more of the nucleotide polymorphisms.

10. The method of claim 9, wherein the nucleotide polymorphisms are selected from the group consisting of the variant nucleotides recited in Table 13.

11. The method of any one of claims 1 to 10, wherein the presence or absence of the allele comprising a “G” at C16759-001-K1 is detected by polymerase chain reaction amplification of a nucleic acid present in the maize plant with a primer configured to specifically amplify a nucleic acid sequence of the allele.

12. The method of claim 11, wherein the primer comprises the sequence AATTATGCTGATGA (SEQ ID NO: 413).

13. A process of selecting a maize plant with anthracnose stalk rot resistance, the process comprising identifying the maize plant according to the process of any one of claims 1 to 12, and selecting the maize plant as having anthracnose stalk rot resistance if the presence or absence of the at least one marker at the resistance locus on chromosome 6 is detected.

14. The process of claim 13, further comprising selecting the maize plant that comprises at least one additional marker allele that is closely linked to and associated with the nucleotide polymorphism(s).

15. The process of claim 14, wherein the additional marker allele is linked to the single nucleotide polymorphism by no more than 2 cM on a single meiosis based genetic map.

16. The process of any one of claims 13 to 15, further comprising selecting the maize plant that comprises at least one additional marker allele that is linked to and associated with the allele comprising a “G” at C16759-001-K1.

17. The process of claim 16, wherein the additional marker allele is linked to the allele comprising a “G” at C16759-001-K1 by no more than 2 cM on a single meiosis based genetic map.

18. The process of any one of claims 13 to 15, further comprising selecting the maize plant that comprises at least one additional marker allele that is linked to and associated with the allele comprising a variant nucleotide polymorphism recited in Table 13.

19. The process of any one of claims 1 to 17, further comprising backcrossing the identified maize plant with another maize plant, preferably comprising backcrossing the resistance locus on chromosome 6 into a genotype which is not NC262A.

20. A method of introgressing an allele associated with anthracnose stalk rot resistance into a maize plant, the method comprising:
a. screening a population with a nucleic acid assay for the detection of at least one marker at a resistance locus on chromosome 6 comprising:
(i) the following single nucleotide polymorphisms
a “C” at C12305-001-K1,
a “C” at C12307-001-K1,
a “G” at C16759-001-K1,
a “G” at C16760-001-K1,
an “A” at C12314-001-K1; and/or
(ii) one or more of the variant nucleotide polymorphisms recited in Table 13; and
b. selecting from the population at least one maize plant comprising the resistance locus on chromosome 6 or comprising a “G” at C16759-001-K1 and/or one or more of the variant nucleotide polymorphisms recited in Table 13; and
c. crossing the at least one maize plant to a second maize plant;
d. evaluating progeny plants for the presence of the “G” at C16759-001-K1 and/or one or more of the variant nucleotide polymorphisms recited in Table 13; and
e. selecting progeny plants possessing the “G” at C16759-001-K1 and/or one or more of the variant nucleotide polymorphisms recited in Table 13.

21. The method of claim 20, wherein the at least one marker is located within 5 cM of the “G” at C16759-001-K1.

22. The method of claim 20, wherein the at least one marker is located within 1 cM of the “G” at C16759-001-K1.

23. The method of claim 20, wherein the resistance locus on chromosome 6 is located on a chromosomal interval between makers PZE-106066805 and PZE-106075546.

24. The method of claim 20, wherein the resistance locus on chromosome 6 is located on a chromosomal interval between 139646631 and 139889078 numbered according to the B73AGPv05 genome sequence.

25. The method of claim 20, wherein the resistance locus of chromosome 6 comprises SEQ ID NO: 272, or a fragment thereof.

26. The method of claim 20, wherein the resistance locus on chromosome 6 comprising one or more nucleotide sequences selected from the group consisting of
ix. a nucleotide sequence of SEQ ID NO:266 or 269,
x. a nucleotide sequence having a coding sequence of SEQ ID NO: 267 or 270,
xi. a nucleotide sequence which is complementary to a sequence from i. or ii.,
xii. a nucleotide sequence which has at least 80% identity with a sequence from i., ii. or iii.,
xiii. a nucleotide sequence which differs from a nucleic acid sequence according to i., ii. or iii. depending on the degeneracy of the genetic code,
xiv. a nucleotide sequence which hybridizes with a nucleic acid sequence according to i., ii. or iii. under stringent conditions,
xv. a nucleotide sequence which encodes for a protein comprising the sequence of SEQ ID NOs: 268 or 271, or
xvi. a nucleotide sequence which encodes for a protein comprising a sequence with at least 80% sequence identity to the sequence of SEQ ID NO: 268 or 271.

27. The method of claim 20, wherein the resistance locus on chromosome 6 is derived from NC262A.

28. A method of selecting a maize plant that displays resistance to anthracnose stalk rot, the method comprising:
a. obtaining a first maize plant that comprises within its genome a haplotype comprising one or more of the following single nucleotide polymorphisms
a “C” at C12305-001-K1,
a “C” at C12307-001-K1,
a “G” at C16759-001-K1,
a “G” at C16760-001-K1,
an “A” at C12314-001-K1; and/or
one or more of a variant nucleotide polymorphism recited in Table 13; and
b. crossing the first maize plant to a second maize plant;
c. evaluating progeny plants for the haplotype in a. or at least one marker allele linked to and associated with the haplotype in b.; and
d. selecting progeny plants that possess the haplotype in a.

29. The method of claim 28, wherein the first maize plant is obtained in (a) that comprises within its genome a haplotype comprising one or more of a “G” at C16759-001-K1, or a variant nucleotide polymorphism recited in Table 13.

30. A nucleic acid molecule comprising one or more nucleotide sequences selected from the group consisting of
ix. a nucleotide sequence of SEQ ID NO: 266, 267, 269, 270 or 272,
x. a nucleotide sequence having a coding sequence of SEQ ID NO: 212 or 215,
xi. a nucleotide sequence which is complementary to a sequence from i. or ii.,
xii. a nucleotide sequence with at least 80% identity to a sequence from i., ii. or iii.,
xiii. a nucleotide sequence which differs from a nucleic acid sequence according to i., ii. or iii. depending on the degeneracy of the genetic code,
xiv. a nucleotide sequence which hybridizes with a nucleic acid sequence according to i., ii. or iii. under stringent conditions,
xv. a nucleotide sequence which encodes for a protein comprising the sequence of SEQ ID NO: 268 or 271, or
xvi. a nucleotide sequence which encodes for a protein comprising a sequence with at least 80% identity to the sequence of SEQ ID NOs: 268 or 271.

31. An expression cassette comprising the nucleic acid molecule of claim 30 operatively linked to heterologous regulatory element, preferably to a heterologous promoter.

32. A method for conferring or increasing resistance to anthracnose stalk rot in a maize plant, the method comprising the following steps:
(d) introducing or introgressing into at least one cell of a maize plant the nucleic acid molecule of claim 30;
(e) optionally regenerating or growing a plant from the at least one cell, and
(f) causing expression of the nucleic acid molecule in the plant.

33. A method for manufacturing a maize plant having anthracnose stalk rot resistance, comprising the following steps:
(a) introducing or introgressing into at least one cell of a maize plant the nucleic acid molecule of claim 30, or the expression cassette of claim 31; or
(b.1) introducing of a site-directed nuclease and a repair matrix into at least one cell of a maize plant, wherein the site-directed nuclease is able to generate at least one double-strand break of the DNA in the genome of the at least one cell and the repair matrix comprises the nucleic acid molecule of claim 30 or a fragment thereof,
(b.2) cultivation of the at least one cell of (b.1) under conditions that allow a homology-directed repair or a homologous recombination, wherein the nucleic acid molecule is integrated from the repair matrix into the genome of the maize plant; and
(c) obtaining the plant having anthracnose stalk rot resistance from the at least one cell.

34. The method according to claim 33, wherein the site-directed nuclease comprises a zinc-finger nuclease, a transcription activator-like effector nuclease, a CRISPR/Cas system, including a CRISPR/Cas9 system, a CRISPR/Cpf1 system, a CRISPR/CasX system, a CRISPR/CasY system, an engineered homing endonuclease, and a meganuclease, and/or any combination, variant, or catalytically active fragment thereof.

35. A maize plant identified according to the process of any one of claims 1-19, or manufactured according to the method of claim 33 or claim 34.

36. A maize plant comprising a resistance locus associated with anthracnose stalk rot resistance, wherein the maize plant is prepared by a process comprising introgressing the resistance locus into the maize plant according to the method of any one of claims 20-27.

37. A maize plant comprising a resistance locus associated with anthracnose stalk rot resistance, wherein the maize plant is prepared by a process comprising introgressing the nucleic acid molecule of claim 30 into the maize plant.

38. A maize plant comprising a resistance locus associated with anthracnose stalk rot resistance, wherein the maize plant is prepared by a process comprising introducing the nucleic acid molecule of claim 30 into the maize plant.

39. A seed or plant part of the maize plant of any one of claims 35-38.
